(19) United States
(12) Patent Application Publication
(10) Pub. No.: US 2024/0000051 A1
(43) Pub. Date: Jan. 4, 2024

(54) INFLUENZA A-RESISTANT ANIMALS HAVING EDITED ANP32 GENES

(71) Applicant: Pig Improvement Company UK Limited, Basingstoke, Hampshire (GB)

(72) Inventors: Brian Burger, Madison, WI (US); Benjamin Beaton, DeForest, WI (US)

(73) Assignee: Pig Improvement Company UK Limited, Basingstoke, Hampshire (GB)

(21) Appl. No.: 18/253,024

(22) PCT Filed: Nov. 15, 2021
(86) PCT No.: PCT/GB2021/052942

§ 371 Date: May 15, 2023

Related U.S. Application Data
(60) Provisional application No. 63/114,084, filed on Nov. 16, 2020.

(51) Int. Cl. A01K 67/027; C12N 15/90; C12N 9/22

(52) U.S. Cl. CPC A01K 67/0275; C12N 15/907; C12N 9/22; A01K 2227/108; A01K 2217/075; C12N 2310/20

(57) ABSTRACT

Ungulate animals and offspring thereof comprising at least one modified chromosomal sequence in at least one gene encoding an ANP32 protein (e.g., a gene encoding an ANP32A protein or an ANP32B protein) are provided. Ungulate cells that contain such modified chromosomal sequences are also provided. The animals, offspring, and cells have increased resistance to type A influenza viruses. Methods for producing pathogen-resistant ungulate animals or lineages of ungulate animals are also provided.

### CROSS REFERENCE TO PRIOR APPLICATION

This application claims the benefit of and priority to U.S. Provisional application No. 63/114,084, filed on Nov. 16, 2020. This application is hereby incorporated by reference in its entirety.

### FIELD

The present disclosure relates to ungulate animals such as pigs and offspring thereof comprising at least one modified chromosomal sequence in at least one gene encoding an ANP32 protein (e.g., in a gene encoding ANP32A or ANP32B). The disclosure further relates to ungulate cells (e.g., porcine cells) comprising at least one modified chromosomal sequence in at least one gene encoding an ANP32 protein. The animals and cells have increased resistance to pathogens, including type A influenza virus. The disclosure further relates to methods for producing pathogen-resistant ungulate animals or lineages of ungulate animals.

### REFERENCE TO SEQUENCE LISTING SUBMITTED ELECTRONICALLY

The official copy of the sequence listing is submitted electronically via EFS-Web as an ASCII-formatted sequence listing with a file named “TD-7-2020-W01-SEQLST.txt” created on Nov. 11, 2021 and having a size of 1,361,770 bytes, and is filed concurrently with the specification. The sequence listing contained in this ASCII-formatted document is part of the specification and is herein incorporated by reference in its entirety.

### BACKGROUND

Influenza A viruses (IAVs) are enveloped, single stranded RNA viruses that cause an acute respiratory disease leading to substantial economic losses to the swine industry each year. To date, three major subtypes of IAVs (H1N1, H1N2, and H3N2) have been identified as being endemic in U.S. swine herds.

IAVs are considered to be one of the most important infectious disease agents affecting North American Swine (Sandbulte et al., 2015). IAVs cause substantial health problems in swine, including high fever, lethargy, anorexia, weight loss, nasal and ocular discharge, cough, sneezing, conjunctivitis, and breathing difficulties (Rajao et al., 2014; CDC, 2014; CFSPH, 2016). The disease progresses rapidly and may be complicated when associated with other respiratory pathogens, leading to pneumonia and severe clinical signs (Rajao et al., 2014). Swine influenza also causes substantial economic losses as a result of weight loss, reduced weight gain, and reproductive failure in infected sows due to high fevers (Rajao et al., 2014).

Moreover, swine IAVs pose a significant zoonotic threat to humans. Variants of the IAVs that normally infect pigs can emerge and cause disease in humans. For example, in spring of 2009, a new swine-origin H1N1 influenza A virus emerged in Mexico and the United States and spread worldwide by human-to-human transmission (Smith et al., 2009). The Centers for Disease Control and Prevention (CDC) estimated that over a one-year period from April 2009 through March 2010, approximately 60 million people were infected, resulting in approximately 12,000 deaths (CDC, 2010).

While vaccination of swine represents one strategy for controlling IAV infection, swine IAV strains are very diverse and prone to mutation and vaccines therefore often have disappointing efficacy in the field (Sandbulte et al., 2015). Furthermore, there has been a dramatic evolutionary expansion in IAV diversity in U.S. swine since 1998, resulting in co-circulation of many antigenically and genetically distinct IAV strains and complicating the control of swine influenza (Rajao et al., 2014; see also CFSPH, 2016). Vaccine efficacy has been compromised by this rapid evolution of influenza viruses, resulting in suboptimal protection against distantly related strains (Rajao et al., 2014). Moreover, vaccines eliminate or reduce clinical signs, but do not always prevent infections or virus shedding, though the amount of virus shed may be reduced (CFSPH, 2016). In addition, vaccine-associated respiratory disease (VAERD), which is characterized by severe respiratory disease, can occur with traditional inactivated vaccines when vaccine virus strains are mismatched with the infecting strain (Rajao et al., 2014).

There is therefore a need in the art for development of additional strategies for the control of IAVs in swine.

### SUMMARY

In general, the present teachings provide for and include a Sus scrofa that comprises an exogenous stop codon in an ANP32 gene, wherein there is reduced or absent ANP32 activity, and wherein the Sus scrofa is resistant to influenza virus. In some embodiments, a Sus scrofa having a genome comprising a genetically edited endogenous ANP32 gene, which can comprise an ANP32A gene or an ANP32B gene, wherein the edited ANP32 gene can comprise a premature stop codon relative to a wild-type gene. In some configurations, the premature stop codon can be upstream of N129 and D130. In various configurations, the genetically edited endogenous ANP32 gene can comprise SEQ ID NO: 7577 or SEQ ID NO: 7578.

In various configurations, the exogenous stop codon confers resistance to an influenza virus. In some configurations, the influenza virus can comprise a type A influenza virus. In various configurations, the type A influenza virus can comprise an H1N1 subtype virus, an H1N2 subtype virus, or an H3N2 subtype virus.

In various configurations, the animal, offspring, or cell can be heterozygous for the genetically edited endogenous ANP32 gene. In various configurations, the animal, offspring, or cell can be homozygous for the genetically edited endogenous ANP32 gene.

Also provided for is a cell isolated from a Sus scrofa edited as described herein.

Also provided for is an isolated cell line obtained from a Sus scrofa of the present teachings. In some configurations, the isolated cell line can be an isolated fibroblast line.

In various embodiments, the present teachings provide for and include a pair of guide RNAs (gRNAs) for editing a Sus scrofa ANP32 gene which can be SEQ ID NO: 33 and SEQ ID NO: 40, SEQ ID NO: 19 and SEQ ID NO: 26, SEQ ID NO: 44 and SEQ ID NO: 46, SEQ ID NO: 55 and SEQ ID NO: 58; or SEQ ID NO: 55 and SEQ ID NO: 59. In some configurations, the gRNAs can be SEQ ID NO: 26 and SEQ ID NO: 19 or SEQ ID NO:55 and SEQ ID NO: 59.

The present disclosure also provides for and includes a method of making a Sus scrofa resistant to an influenza virus which can comprise: introducing into a Sus scrofa MIT oocyte or zygote a CAS protein and a pair of gRNAs that introduce a premature stop codon into an endogenous ANP32 gene, wherein a stop codon can be introduced into an endogenous ANP32 gene of the oocyte or zygote; implanting an embryo obtained from the oocyte or zygote into a recipient female such that a Sus scrofa can be obtained from the implanted embryo, wherein the Sus scrofa obtained can be heterozygous for the ANP32 gene comprising a premature stop codon; breeding the heterozygous Sus scrofa to a Sus scrofa of opposite sex that can also comprise a premature stop codon in the ANP32 gene; selecting offspring from the breeding that are homozygous for the premature stop codon in the ANP32 gene, wherein these homozygous offspring are resistant to influenza. In some configurations, the pair of gRNAs can comprise sequences consisting of SEQ ID NO: 26 and SEQ ID NO: 19 or SEQ ID NO: 55 and SEQ ID NO: 59. In various configurations, the CAS protein and the gRNAs can be introduced as a pre-formed ribonucleoprotein (RNP) complex. In various configurations, the premature stop codon comprises the nucleic acid sequence consisting of SEQ ID NO: 7577 or SEQ ID NO: 7578.

The present disclosure also provides for and includes a method of making a Sus scrofa that can be resistant to an influenza virus comprising: introducing into a Sus scrofa MII oocyte or zygote a CAS protein and a pair of gRNAs that can introduce a premature stop codon into an endogenous ANP32 gene, wherein a stop codon can be introduced into an endogenous ANP32 gene of the oocyte or zygote; implanting an embryo obtained from the oocyte or zygote into a recipient female such that a Sus scrofa can be obtained from the implanted embryo, wherein the Sus scrofa obtained can be homozygous for the ANP32 gene comprising a premature stop codon; wherein the homozygous Sus scrofa can be resistant to influenza. In some configurations, the pair of gRNAs can comprise sequences consisting of SEQ ID NO: 26 and SEQ ID NO: 19 or SEQ ID NO: 55 and SEQ ID NO: 59. In various configurations, the CAS protein and the gRNAs can be introduced as a pre-formed ribonucleoprotein (RNP) complex. In various configurations, the premature stop codon comprises the nucleic acid sequence consisting of SEQ ID NO: 7577 or SEQ ID NO: 7578.

The present teachings also provide for and include ANP32 gene edited to confer Influenza resistance in Sus scrofa wherein the edit introduces an exogenous stop codon and the edited ANP32 gene comprises SEQ US NO: 7577 or SEQ ID NO: 7578. In some configurations, the edit is created using SEQ ID NO: 26 and SEQ ID NO: 19 or SEQ ID NO: 55 and SEQ ID NO:59.

In various embodiments, the present teachings provide for and include a non-reproductive Sus scrofa gene comprising an ANP32 gene of the present teachings. In various configurations, the present teachings also provide for cell line comprising a plurality of the non-reproductive ANP32 cell. In some configurations, the cell line can be a fibroblast cell line.

Ungulate animals and offspring thereof are provided. The animals and offspring comprise at least one modified chromosomal sequence in at least one gene encoding an ANP32 protein.

Ungulate cells are also provided. The cells comprise at least one modified chromosomal sequence in at least one gene encoding an ANP32 protein.

A method for producing an ungulate animal or lineage of ungulate animals having reduced susceptibility to a pathogen is provided. The method comprises modifying an ungulate oocyte or an ungulate sperm cell to introduce a modified chromosomal sequence in a gene encoding an ANP32 protein into at least one of the oocyte and the sperm cell, and fertilizing the oocyte with the sperm cell to create a fertilized egg containing the modified chromosomal sequence in the gene encoding the ANP32 protein. The method further comprises transferring the fertilized egg into a surrogate female ungulate animal, wherein gestation and term delivery produces a progeny animal.

Another method for producing an ungulate animal or lineage of ungulate animals having reduced susceptibility to a pathogen is provided. The method comprises modifying an ungulate fertilized egg to introduce a modified chromosomal sequence in a gene encoding an ANP32 protein into the fertilized egg. The method further comprises transferring the fertilized egg into a surrogate female ungulate animal, wherein gestation and term delivery produces a progeny animal.

A further method for producing an ungulate animal or a lineage of ungulate animals having reduced susceptibility to infection by a pathogen is provided. The method comprises enucleating an ungulate oocyte, modifying a donor ungulate somatic cell to introduce a modified chromosomal sequence into a gene encoding an ANP32 protein, fusing the oocyte with the modified donor ungulate somatic cell, and activating the oocyte to produce an embryo.

A method of increasing an ungulate animal's resistance to infection with a pathogen is provided. The method comprises modifying at least one chromosomal sequence in at least one gene encoding an ANP32 protein, so that production or activity of the ANP32 protein is reduced, as compared to production or activity of the same ANP32 protein in an ungulate animal that does not comprise a modified chromosomal sequence in the gene encoding the ANP32 protein.

In any of the ungulate animals, offspring, cells, or methods described herein, the gene encoding the ANP32 protein can be a gene encoding an ANP32A protein or a gene encoding an ANP32B protein.

A population of ungulate animals is provided. The population comprises two or more of any of the ungulate animals and/or offspring thereof described herein.

Another population of ungulate animals is provided. The population comprises two or more animals made by any of the methods described herein and/or offspring thereof.

Guide RNAs (gRNAs) are provided. The gRNA can comprise a nucleotide sequence of any one of SEQ ID NOs. 15-7,576.

Other objects and features will be in part apparent and in part pointed out hereinafter.

### DETAILED DESCRIPTION

The acidic (leucine-rich) nuclear phosphoprotein 32 kDa (ANP32) family of proteins is made up of small, evolutionarily conserved proteins characterized by an amino-terminal leucine-rich repeat domain and a carboxy-terminal low-complexity acidic region (Reilly et al., 2014). The mammalian members of the ANP32A family—ANP32A, ANP32B, and ANP32E—have physiologically diverse functions, including chromatin modification and remodeling, apoptotic caspase modulation, protein phosphatase inhibition, transcription regulation, and regulation of intracellular transport (Reilly et al., 2014; Staller et al., 2014). In addition, ANP32 proteins have been found to be dysregulated in an array of cancers (Reilly et al., 2014). ANP32 proteins interact with the IAV polymerase and are needed for viral replication. In human cells, an ANP32A/ANP32B double knockout nearly completely eliminated viral polymerase activity and viral replication (Staller et al., 2014).

The Sus scrofa (pig) genome contains several homologs of human ANP32A, including ANP32A and ANP32B (also referred to as NANS). Porcine ANP32A and ANP32B genes encode asparagine (N) at position 129 (N129) and aspartic acid (D) at position 130 (D130), amino acids that have been identified as being required for interaction of human and chicken ANP32 proteins with the influenza A viral polymerase (Staller et al., 2019; Baker et al., 2019). As described further hereinbelow, in order to generate IAV-resistant pigs, the present inventors designed editing strategies to modify porcine ANP32A and ANP32B genes. For example, editing strategies were designed to introduced stop codons in conserved exonic sequences of the porcine ANP32A and ANP32B genes upstream of the regions of the genes coding for N129 and D130. Examples of sequences edited to comprise the exogenous stop codons include SEQ ID NOs: 7577 and 7578

Accordingly, the present invention is directed to ungulate animals and offspring thereof comprising at least one modified chromosomal sequence in at least one gene encoding an ANP32 protein. The invention further relates to ungulate cells comprising at least one modified chromosomal sequence in at least one gene encoding an ANP32 protein. The animals and cells have increased resistance to pathogens, including influenza A viruses.

The animals and cells have chromosomal modifications (e.g., insertions, deletions, or substitutions) that inactivate or otherwise modulate ANP32A and/or ANP32B gene activity. Since ANP32 proteins are needed for viral replication of IAVs, the animals and cells having modified ANP32 genes display resistance to IAVs when challenged.

Populations of any of the animals described herein are also provided.

The present invention is further directed to methods for producing pathogen-resistant ungulate animals and lineages of such animals. The methods comprise introducing a modified chromosomal sequence into a gene encoding an ANP32 protein.

Guide RNAs for use in creating the animals and cells are also provided.

The animals, offspring, cells, populations of animals, methods, and guide RNAs are further described hereinbelow.

### Definitions

When introducing elements of the present invention or the preferred embodiments(s) thereof, the articles “a,” “an,” “the,” and “said” are intended to mean that there are one or more of the elements.

The term “and/or” means any one of the items, any combination of the items, or all of the items with which this term is associated.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The terms “comprising,” “including,” and “having” are intended to be inclusive and mean that there may be additional elements other than the listed elements. The term “CRISPR” stands for “clustered regularly interspaced short palindromic repeats.” CRISPR systems include Type I, Type II, and Type III CRISPR systems.

The term “Cas” refers to “CRISPR associated protein.” Cas proteins include but are not limited to Cas9 family member proteins, Cas6 family member proteins (e.g., Csy4 and Cas6), Cas5 family member proteins, and Cas12 family member proteins.

The term “Cas9” can generally refer to a polypeptide with at least about 5%, 10%, 20%, 30%, 40%, 50%, 60%, 70%, 80%, 90%, 95%, 98%, 99%, 99.5%, 99.9% or 100% sequence identity and/or sequence similarity to a wild-type Cas9 polypeptide (e.g., Cas9 from S. pyogenes). Illustrative Cas9 sequences are provided by SEQ ID NOs. 1-256 and 795-1346 of U.S. Patent Publication No. 2016/0046963. SEQ ID NOs. 1-256 and 795-1346 of U.S. Patent Publication No. 2016/0046963 are hereby incorporated herein by reference. “Cas9” can refer to can refer to a polypeptide with at most about 5%, 10%, 20%, 30%, 40%, 50%, 60%, 70%, 80%, 90%, 95%, 98%, 99%, 99.5%, or 99.9%, 100% sequence identity and/or sequence similarity to a wild type Cas9 polypeptide (e.g., from S. pyogenes). “Cas9” can refer to the wild-type or a modified form of the Cas9 protein that can comprise an amino acid change such as a deletion, insertion, substitution, variant, fusion, chimera, or any combination thereof.

The term “Cas5” can generally refer to can refer to a polypeptide with at least about 5%, 10%, 20%, 30%, 40%, 50%, 60%, 70%, 80%, 90%, 95%, 98%, 99%, 99.5%, 99.9%, or 100% sequence identity and/or sequence similarity to a wild type illustrative Cas5 polypeptide (e.g., Cas5 from D. vulgaris). Illustrative Cas5 sequences are provided in FIG. 42 of U.S. Patent Publication No. 2016/0046963. FIG. 42 of U.S. Patent Publication No. 2016/0046963 is hereby incorporated herein by reference. “Cas5” can generally refer to can refer to a polypeptide with at most about 5%, 10%, 20%, 30%, 40%, 50%, 60%, 70%, 80%, 90%, 95%, 98%, 99%, 99.5%, 99.9%, or 100% sequence identity and/or sequence similarity to a wild-type Cas5 polypeptide (e.g., a Cas5 from D. vulgaris). “Cas5” can refer to the wild-type or a modified form of the Cas5 protein that can comprise an amino acid change such as a deletion, insertion, substitution, variant, fusion, chimera, or any combination thereof.

The term “Cas6” can generally refer to can refer to a polypeptide with at least about 5%, 10%, 20%, 30%, 40%, 50%, 60%, 70%, 80%, 90%, 95%, 98%, 99%, 99.5%, 99.9%, or 100% sequence identity and/or sequence similarity to a wild type illustrative Cas6 polypeptide (e.g., a Cas6 from T. thermophilus). Illustrative Cas6 sequences are provided in FIG. 41 of U.S. Patent Publication No. 2016/0046963. FIG. 41 of U.S. Patent Publication No. 2016/0046963 is hereby incorporated herein by reference. “Cas6” can generally refer to can refer to a polypeptide with at most about 5%, 10%, 20%, 30%, 40%, 50%, 60%, 70%, 80%, 90%, 95%, 98%, 99%, 99.5%, 99.9%, or 100% sequence identity and/or sequence similarity to a wild-type Cas6 polypeptide (e.g., from T. thermophilus). “Cas6” can refer to the wildtype or a modified form of the Cas6 protein that can comprise an amino acid change such as a deletion, insertion, substitution, variant, fusion, chimera, or any combination thereof.

The terms “CRISPR/Cas9” or “CRISPR/Cas9 system” refer to a programmable nuclease system for genetic editing that includes a Cas9 protein, or derivative thereof, and one or more non-coding guide RNAs (“gRNAs”) that provide the function of a CRISPR RNA (crRNA) and trans-activating RNA (tracrRNA) for the Cas9. The crRNA and tracrRNA can be separate RNA molecules or can be combined into a single RNA molecule to produce a “single guide RNA” (sgRNA). The crRNA or the cRNA portion of the sgRNA provide sequence that is complementary to the genomic target.

“Disease resistance” is a characteristic of an animal, wherein the animal avoids the disease symptoms that are the outcome of animal-pathogen interactions, such as interactions between a porcine animal and an influenza A virus. That is, pathogens are prevented from causing animal diseases and the associated disease symptoms, or alternatively, a reduction of the incidence and/or severity of clinical signs or reduction of clinical symptoms. One of skill in the art will appreciate that the compositions and methods disclosed herein can be used with other compositions and methods available in the art for protecting animals from pathogen attack.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

As used herein, “gene editing,” “gene edited”, “genetically edited” and “gene editing effectors” refer to the use of homing technology with naturally occurring or artificially engineered nucleases, also referred to as “molecular scissors,” “homing endonucleases,” or “targeting endonucleases.” The nucleases create specific double-stranded chromosomal breaks (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), Clustered Regularly Interspaced Short Palindromic Repeats (CRISPR) systems (e.g., CRISPR/Cas9 systems), and meganucleases (e.g., meganucleases re-engineered as homing endonucleases). The terms also include the use of transgenic procedures and techniques, including, for example, where the change is a deletion or relatively small insertion (typically less than 20 nt) and/or does not introduce DNA from a foreign species. The term also encompasses progeny animals such as those created by sexual crosses or asexual propagation from the initial gene edited animal.

The terms “genome editing,” “genetic editing,” and “genetically edited,” can refer to altering the genome by deleting, inserting, substituting, or otherwise altering specific nucleic acid sequences. The altering can be gene or location specific. Genome editing can use nucleases to cut a nucleic acid thereby generating a site for the edit. Editing of non-genomic nucleic acid is also contemplated. A protein containing a nuclease domain can bind and cleave a target nucleic acid by forming a complex with a nucleic acid-targeting nucleic acid. In one example, the cleavage can introduce double stranded breaks in the target nucleic acid. A nucleic acid can be repaired e.g., by endogenous non-homologous end joining (NHEJ) machinery or homology directed repair (HDR) machinery. The former does not rely on a template DNA to repair and can often lead to insertions or deletions in the target nucleic acid. HDR requires a template DNA and therefore can result in higher fidelity repair and allow for substitutions (e.g., single nucleotide substitutions) in the target nucleic acid with minimal disruption in surrounding areas. In a further example, a piece of nucleic acid can be inserted. Modifications of nucleic acid-targeting nucleic acids and site-directed polypeptides can introduce new functions to be used for genome editing.

As used herein “homing DNA technology,” “homing technology” and “homing endonuclease” include any mechanisms that allow a specified molecule to be targeted to a specified DNA sequence including Zinc Finger (ZF) proteins, Transcription Activator-Like Effectors (TALEs), meganucleases, and CRISPR systems (e.g., CRISPR/Cas9 systems).

The terms “increased resistance” and “reduced susceptibility” herein mean, but are not limited to, a statistically significant reduction of the incidence and/or severity of clinical signs or clinical symptoms which are associated with infection by pathogen. For example, “increased resistance” or “reduced susceptibility” can refer to a statistically significant reduction of the incidence and/or severity of clinical signs or clinical symptoms which are associated with infection by influenza A in an animal comprising a modified chromosomal sequence in an ANP32 gene as compared to a control animal having an unmodified chromosomal sequence. The term “statistically significant reduction of clinical symptoms” means, but is not limited to, the frequency in the incidence of at least one clinical symptom in the modified group of subjects is at least 10%, preferably at least 20%, more preferably at least 30%, even more preferably at least 50%, and even more preferably at least 70% lower than in the non-modified control group after the challenge with the infectious agent.

“Knock-out” means disruption of the structure or regulatory mechanism of a gene. Knock-outs may be generated through homologous recombination of targeting vectors, replacement vectors, or hit-and-run vectors or random insertion of a gene trap vector resulting in complete, partial or conditional loss of gene function.

Herein, “reduction of the incidence and/or severity of clinical signs” or “reduction of clinical symptoms” means, but is not limited to, reducing the number of infected subjects in a group, reducing or eliminating the number of subjects exhibiting clinical signs of infection, or reducing the severity of any clinical signs that are present in one or more subjects, in comparison to infection of wild-type subjects. For example, these terms encompass any clinical signs of infection, lung pathology, viremia, antibody production, reduction of pathogen load, reduction in pathogen shedding, reduction in pathogen transmission, or reduction of any clinical sign symptomatic of influenza A. Preferably these clinical signs are reduced in one or more animals of the invention by at least 10% in comparison to subjects not having a modification in the ANP32 gene and that become infected. More preferably clinical signs are reduced in subjects of the invention by at least 20%, preferably by at least 30%, more preferably by at least 40%, and even more preferably by at least 50%.

References herein to a deletion in a nucleotide sequence from nucleotide x to nucleotide y mean that all of the nucleotides in the range have been deleted, including x and y. Thus, for example, the phrase “a 100 base pair deletion from nucleotide 1,000 to nucleotide 1,099 as compared to SEQ ID NO: X” means that each of nucleotides 1,000 through 1,099 have been deleted, including nucleotides 1,000 and 1,099.

“Resistance” of an animal to a disease is a characteristic of an animal, wherein the animal avoids the disease symptoms that are the outcome of animal-pathogen interactions, such as interactions between a porcine animal and influenza A. That is, pathogens are prevented from causing animal diseases and the associated disease symptoms, or alternatively, a reduction of the incidence and/or severity of clinical signs or reduction of clinical symptoms. One of skill in the art will appreciate that the methods disclosed herein can be used with other compositions and methods available in the art for protecting animals from pathogen attack.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Zinc finger and TALE binding domains can be “engineered” to bind to a predetermined nucleotide sequence, for example via engineering (altering one or more amino acids) of the recognition helix region of naturally occurring zinc finger or TALE proteins. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; WO WO 96/06166; WO 98/53057; WO 98/54311; WO 00/27878; WO 01/60970 WO 01/88197, WO 02/099084 and U.S. Publication No. 20110301073.

The term “breeding” as used herein refers to a process comprising the selection of superior male and superior female animals use for creation of the next generation of offspring. This process further comprises the union of male and female gametes so that fertilization occurs. Such a union may be brought about by mating (copulation) or by in vitro or in vivo artificial methods. Such artificial methods can include, but are not limited to, artificial insemination, surgical assisted artificial insemination, in vitro fertilization, intracytoplasmic sperm injection, zona drilling, in vitro culture of fertilized oocytes, ovary transfer, and ovary splitting. The term “breeding” as used herein also can include transferring of a fertilized oocyte into the reproductive tract of a female animal in order to allow for more offspring from a particular elite female.

As used herein, “ANP32” can refer to an ANP32A or ANP32B gene or protein. Skilled persons will be able to determine whether a gene or protein is being referred to by the context of the reference.

Various other terms are defined hereinbelow.

### Animals and Cells Having a Modified Chromosomal Sequence in a Gene Encoding an ANP32 Protein

Described herein are ungulate animals and offspring thereof, and ungulate cells comprising at least one modified chromosomal sequence in at least one gene encoding the ANP32 protein. For example, the animals, offspring, or cell can have an insertion or deletion (“INDEL”) which confers improved or complete resistance to infection by a pathogen (e.g., an influenza A virus).

The ungulate animal or offspring can be selected from the group consisting of pigs, cattle, horses, sheep, goats, buffalo, bison, alpacas, llamas, yaks, deer, elk, and camels. Likewise, the ungulate cell can be derived from an animal selected from the group consisting of pigs, cattle, horses, sheep, goats, buffalo, bison, alpacas, llamas, yaks, deer, elk, and camels.

For example, the ungulate animal or offspring can be pigs or cattle (e.g., beef or dairy cattle). The ungulate cell can be a porcine cell or a bovine cell.

The ungulate animal or offspring can be a pig. The ungulate cell can be a porcine cell.

Porcine ANP32A has two isoforms, the complete nucleotide sequences of which can be found in the Ensembl database under Accession Numbers ENSSSCT00000005475.4 and ENSSSCT00000070641.1. Both of the porcine ANP32A isoforms align with human ANP32A, but the porcine ANP32A isoform encoded by ENSSSCT00000005475.4 aligns more closely with human ANP32A. The complete nucleotide sequence for ENSSSCT00000005475.4 is provided herein as SEQ ID NO: 1. SEQ ID NO: 1 is the sequence for the “top” strand, and ANP32A is encoded on the complementary “bottom” strand. Accordingly, for ease of reference, the reverse complement of the “bottom” coding strand was generated, so that the sequence could be read from left to right. This reverse complement sequence is provided as SEQ ID NO: 2. In addition, a partial version of SEQ ID NO: 2 is provided as SEQ ID NO: 3. SEQ ID NO: 3 is used as a reference sequence herein. SEQ ID NO: 3 includes nucleotides 2,588-34,583 of SEQ ID NO: 2, corresponding to exon 1, intron 1, exon 2, intron 2, exon 3, intron 3, and exon 4 of the ANP32A gene. Table 1 provides the locations of the exons and introns of the ANP32A gene in SEQ ID NOs. 2 and 3.

TABLE 1
Locations of exons and introns of the
ANP32A gene in SEQ ID NOs. 2 and 3

| | Location in SEQ ID NO: | Location in SEQ ID NO: |
|---|---|---|
| | 2 (nucleotides) | 3 (nucleotides) |
| Exon 1 | 2,588-2,785* | 1-198* |
| Intron 1 | 2,786-32,017 | 199-29,430 |
| Exon 2 | 32,018-32,167 | 29,431-29,580 |
| Intron 2 | 32,168-32,305 | 29,581-29,718 |
| Exon 3 | 32,306-32,428 | 29,719-29,841 |
| Intron 3 | 32,429-34,384 | 29,842-31,797 |
| Exon 4 | 34,385-34,583** | 31,798-31,996** |
| Intron 4 | 34,584-35,763 | — |
| Exon 5 | 35,764-35,861 | — |
| Intron 5 | 35,862-38,019 | — |
| Exon 6 | 38,020-38,083 | — |
| Intron 6 | 38,084-38,328 | — |
| Exon 7 | 38,329-39,564 | — |

*Exon 1 of the ANP32A gene includes a 5′ untranslated region (5′-UTR) at nucleotides 2,588-2,731 of SEQ ID NO: 2 and nucleotides 1-144 of SEQ ID NO: 3. The start codon is at nucleotides 2,732-2,734 of SEQ ID NO: 2 and nucleotides 145-147 of SEQ ID NO: 3.
**Exon 4 of the ANP32A gene includes the nucleotides that encode asparagine 129 (N129) and aspartic acid 130 (D130). These amino acids are encoded by nucleotides 34,442-34,447 of SEQ ID NO: 2 and nucleotides 31,855-31,860 of SEQ ID NO: 3.

Table 2 provides an annotated version of SEQ ID NO: 3, showing the locations of exons 1, 2, 3, and 4. Exons 1, 2, 3, and 4 are shown in underlined text. Coding sequence is both underlined and bold, while untranslated sequence that is part of exon 1 is underlined only. The codons that encode asparagine 129 (N129) and aspartic acid 130 (D130) are shown in uppercase text.

TABLE 2
Annotated version of reference sequence SEQ ID NO: 3 (partial ANP32A
gene sequence)
SEQ ID NO: 3

| | |
|---|---|
| 1 | agtgtgcgcc gcgggtgctg ggggctcgag aaccgagcgg agctggttga gccttcaaag |
| 61 | tcctaaaacg cgcggccgtg ggttcggggt ttattgattg aattccgccg gcgcgggagc |
| 121 | ctctgcagag agagagcgcg agagatggag atggacaaac ggattcattt agagctgcgg |
| 181 | aacaggacgc cctctgatgt gagcatttgc caaaaatatc tctgtcggtc cattctccta |
| 241 | ctttgtgtgt gtgtgtgtgc gttgggggga ggcacttttt ttgggggggg cgcctaggag |
| 301 | atgtgggttt gggggtcgga ctgcaaatga attgcaaggt tggggggttg caatgccatc |
| 361 | cgatcatttt ctggctgtcg ccgccgctgc ccttggtgtg tgactacctg tgcgtgtgct |
| 421 | cccgcgtgtg cgagtgtggg tgcgagagtg tgcgtgcgag tgtgcaccca tcgggggcca |
| 481 | gcgggcgcac gcgtttgggt cggagagaat gaggcagaat aaccctctga gctcgctgct |
| 541 | cgcgtgactc cgtctagggg aaagattaac gagggaggag gaggagaggg gctgcctcct |
| 601 | ggatttaaca acaaaaaaag ggggttttat attttataaa tgaatccccc cttcctcccc |
| 661 | cctcccgcgc cttccccctc cctttctccc tctctctccg tctctctctc tctctctctc |
| 721 | gctcgcacac acacaataag ttttgggctt cgcgagtccg gtttagtttc tgataaatgt |
| 781 | gggccgaggc ccctggggag agcgcatggg gagggggctg cagcggagtt gggaatggct |
| 841 | cgagtttaaa gtgcgtcttc atttctttgc agtgatgagg cgagagacgg gggagccata |
| 901 | tttgtaactt tgtagtgttc gtgaacgcgc cctttctctc cctccctccc tttctctgcc |
| 961 | tcctttcctc tectcctctt tctccctcac tctttccacc ccaccttcgg gccagatgct |
| 1021 | gatgtttctt cctttccttc tgaagggaag ataaattgga agaagggggg ggagggattt |
| 1081 | tcgaacgtgc aaaggcacag ataatccccg cgcctcccca cctcgcgcga tcgctactgc |
| 1141 | aagttaccca aaggctaagt ccaggtcctc cctctccccc ttgctccttc cagcctccct |
| 1201 | aaggtgccag ccttcgagcc gtggcgcccc ccggcaccga cccctggcct gtcttgtctc |
| 1261 | tagggtgggc tccttggcag ccattcggcg acgggttaat aaatgggccc gggaaacttt |
| 1321 | gtcaggacgc gagctgccca gagctggtgc ccgtggtggg tattggctgg gtccagattt |
| 1381 | atgcgggcat aacagacacg cagctccggg atgggagttt gccttcccgt cgccgttatt |
| 1441 | gattgggcag aggcgcgagt acggccagag gaaacttaca cggatgggaa cccggaggct |
| 1501 | ctggccaaag cccaccccta aggaggggaa aggtgagggt gggttactcg aacgaacacc |
| 1561 | cgtttttatt tctgtctccc tctcgctttc agtgttttat gtgagttggc ggctcagcgg |
| 1621 | aggagaccca gggtctgggg tggagacgct ggccgactgc cctcgtggca cctcccggga |
| 1681 | caagcggagc gcgggcggag gaaccccgca cccccagccc gggtggccgg ggttccacag |
| 1741 | agttgggagt ttgggtagca ctgtgcgctg acctgtcgcc gcttcagccc gttaaaaccc |
| 1801 | gaatccctgg cggaggcagg ccttgcttcc tcttttctct agttcctccc ccaaacctga |
| 1861 | gtgagacttt tgaaaacaag atcctttttg tgccccccca tcataacgca gctgtggact |
| 1921 | gtcgacgtgc agtgccttat ggcgcgcagg gcggggaggg ggtggcggag ggaggggagg |
| 1981 | ggttgcccgc ccgaggacgc aggagtttcc gcgaacaatt tgccggcggt ttgcctttct |
| 2041 | ctggccggtt tcatcccagc ttggacccgg agccggagga ctggggggct gggtgtggtg |
| 2101 | atgggggagg gggcccgagc cgcgggcttc ccgcgtactt tccggaagcc ccccgtgcgg |
| 2161 | agggggaagg gcgccgagta gggggtcggg gtcgcggcgg gagggtgtgg aggcgcggtt |
| 2221 | cgcccggcct accccatccc cgctgctggc caactgggag cgagatcgaa agcgggtcga |
| 2281 | accggctctt taaacttcgc cgagtgtgtg ctgggggagg ggagcgcgcc gcgccgaact |
| 2341 | gggagcgatt tctcacgctt cgccccctcg cctccgagcc tcccggccca cacgcctgcc |
| 2401 | ccctgccttg aggcggggtg gggcgcgggt gagggtggag ggggcacttt ctgcccgttt |
| 2461 | ggggaggagg gggagaaagc tagctaatga gggacaaaag gggtggggga ggagaaactg |
| 2521 | gtctttttaa acgttcttaa gataaatagc tcccggagaa ctccccctgc gcccctcccc |
| 2581 | gccgtgctct cccccgggtc ccctcacacc caccttcgga gcctctccca actctgatcc |
| 2641 | gagggccggt ggccacgccg cgacgccgaa ataggacgag cagccatgtt tcccgggtct |
| 2701 | cgcgcggccc gccccgcccc tcccccatgg cgcggccggc gccgagcgcg gggctccgaa |
| 2761 | tgcgggcgcc aggctccatg tggcgcgggg tgcgggctgg ttggcaggcg accgggtggc |
| 2821 | ctggccctgt gcgcgcaggt cgggtcgcgg ccgcgccggg ctggcccccg ggcgggcctt |
| 2881 | ttggctttcc gagctggttt tgagctcaag gtgttttatt tattattgtt gtttttttaa |
| 2941 | cggccgaagc cgcccgaggc cgaatccgaa ggggcgccga ggcgttatgg ggggggcagg |
| 3001 | gacggccgcc agggtcctgg tcgcctgtgg cacgactgct gggcgccggg ctgccatttt |
| 3061 | gtcaccgtgg ctgttgtcgc tgttgggggg gggacgcaga ctcttgcgcc atcgccccga |
| 3121 | cggccccgga cctggagaca gcagggggag ccggtcccag ggaccacccc ccccagttgt |
| 3181 | actctgggcc cccatgccgg tcccctccct gtgccaactg cctgaggctc tgctggagcg |
| 3241 | ccgcggcggc cgtggctccg tagggtgacg atcctccctg cgagtgctcc ccccaccccc |
| 3301 | cggcgcgttc tctgcctccc ccgcccctct tctcgccagt atctcgccct tcctcctgca |
| 3361 | ggcgttttag ccgtagttaa atgtggccct aaagacgaac ggatcgccct ggtgtttgct |
| 3421 | ctttggcgga agtgatagga gggtgggggg agaaattaaa caagaggttt cttttggggg |
| 3481 | aggcgggtgc gcacgcgact ctagcggccc tgactgaagc caagaaggca gccgtttacc |
| 3541 | cgggagggac gagttgatgc gttggggaaa cgccctcagg ggctgggctc tccgggaagg |
| 3601 | taggtcgcgc tccgcgtttc aggtgggtgt tgcagcgccc tgctggctgc taggggaggc |
| 3661 | ccccttgctc tttttctgct cctccccctt ttatttgtgc ccaagggaag gcagctacag |
| 3721 | tagtaaaagt aaccctaaac ggaaaagaat agggagtaag gtttgccttt taaaaagtaa |
| 3781 | atgtcttttt ccaaccacct ttctctggct cttgactcca gagccaggaa gggcatcgcg |
| 3841 | tcgctgaagt ctctctggga ctttttaaaa gttgatatgg atgctggtta agcacctcat |
| 3901 | tggttaaact ctactagacg tctgcatgga gactttcaag gcagtgcccg actaaggctg |
| 3961 | cccagaaagt aaggtttaac tttgaccttt ggaatacatc gcatgatgtt ttccagactg |
| 4021 | acttactgcc taattgggtt caagtagcct gatattaact ccagtttatg caagcctacc |
| 4081 | agggatccag gggcagaaaa ttgagcatga gttccctata gtcatggtca gtactgtttt |
| 4141 | gttactggat tcccgttctt gatggtagtg ttgagcctga ggggttcaca gtgaagctgg |
| 4201 | gtagtggaac catgcaggcc tgagtttgaa tgcttcctgc cacaaaagat aagttaacct |
| 4261 | gcctctttga tccttgattt cctcgcttgt aaaatgggac taataatctc atttaatgag |
| 4321 | tcagtgacaa aaccggcctg atctgtccct cgggtgctca gtgtgtgata gtgttattct |
| 4381 | cagagagata ttggagtgtg atgaggcaca actttgagct tttctagggt ttagagaaaa |
| 4441 | acaacaatag ctggtgctta tatagtaccc atgctatgcc tggggcagtt gtaagtggat |
| 4501 | tacacaaatc aattaatttg gtccccacag cagcactgtg aggcaggtac tcctattatc |
| 4561 | cttatatcac acataggaga ttgaggcata aagatgttat cacaggcagt ctgcatactc |
| 4621 | aacctgcact ctttactgcc tcccctgaag cccccttatt tgtaatatac ggtgggaaag |
| 4681 | agctttttct ttggctttaa aatggagcca ccttatgaga gagaagcctg ggtagcctct |
| 4741 | gggttgaaga tactgaggta ggtggtcagg atcagagact ctgtggattg cggttttaag |
| 4801 | ggggaagcca tattacactt tcctactttg ggttagcaca acacatctgg ttaggctgaa |
| 4861 | atgaatctgg ccttttaggt gtcgggggac acattttgag taatgagaag ataagcagga |
| 4921 | gccactcagt ggtgtgtatg ggatcatgca tgtaatgtgt tttccacttt catcagtctc |
| 4981 | catctacaga ccaaaagctg aacagaattg gcatgtcctt agtttctagg acctagttta |
| 5041 | ccaataacac tttatggatc atctcactta atcctccccc aactcctcaa gggtaggcca |
| 5101 | ggcaaggcta tgtcgtctcg gttgcagggg tgaatcatcc aggttgggag agaaggtcag |
| 5161 | acagccagta ctcttcactg ttgggttccc ccattgtaaa acagtaggga ctttgaaaat |
| 5221 | tggatagact atattgggtc ctcagtggcc tcttaccaga atcacctggg aagcatgtag |
| 5281 | aatgcaggtt cctggctcca ctgctaggca ttggtttagt actggcattg ccaaattttg |
| 5341 | ggtgagctgc ctgtttttgt atcatgagct aagaataatt aattagtacg tgtaaacatt |
| 5401 | gcaatcaatt tgatgatgtg gaacactttg aatcccaatt aagtgaaatg ttattatccc |
| 5461 | ccttccccct gcaaagaatt cttatctcct tagaccttta ttaccaaaaa aattgtactt |
| 5521 | attatatatt ttgtatttta tcaataattg tagaaatgtg ttttcttaca ataaaaaaag |
| 5581 | tgcctatgta gtactgtcaa tattacctct aggtaaagtt tactgtttgg ctctttacag |
| 5641 | aagtttgata atctctggtt tagtagattg tggggaagga ctttgggatc tgggttttta |
| 5701 | tttttatttt atatttttat ctttttaggg atgcaccctc tgcatatgga gattcccagg |
| 5761 | ctaggggtcg aatcagagct atagctgcca gcctacacca cagccacagc aacaccaggt |
| 5821 | ctgagccatg tctgcaacct acaccacagc tcacagcaat gctggatcct tatgccactg |
| 5881 | agcaaggcca gggatcaaag ctgcatcctc atggatacta gtcatattca tttttgctga |
| 5941 | gccatgatgg gaactcccta aatttttttt tttaattgtt aaaatttttt aaagcatgtg |
| 6001 | acaagccttt ctttttggtg gtgcttaaag ggggtctttc caaggccttt tacatgtgcc |
| 6061 | ctttctctag ttgatatgta gtggaaattg gcttcaatcc atgaacatcc ataagaagag |
| 6121 | aagtagtact tactgctggt gtaaaattga gcattccttc tattcagaag ttattctctg |
| 6181 | taaccaatgg aaatttttta tctttcatat tcaaaatatt tcaaaaggct tttgtatatc |
| 6241 | ttgtagttaa atggttaccc ttaagattta gatgtcttca aaactagagg gaaggacttt |
| 6301 | gaactgcaac agtttgagct ttgaggtcca gaaggtgcag tagctgtgta cctggtgact |
| 6361 | gacatcagac tctcagagca cagaccattg aaaacctgcg gataatcctg gaatctctag |
| 6421 | tttgggcctg ctcctcttcc accagggcat attgcctggg gaatgtacca agagtaatgt |
| 6481 | ggctttgagt tccagagccc ttctttcaac ttctagtctc ttgtgcctga gctaatgagt |
| 6541 | ggaagaaata gatgtacgat tggcctggcc aggaaggagg caataaatct aaggggagca |
| 6601 | ggcccagtca ccataactgt ggcctgtact ttacagcaaa ccacagaccc agttccctct |
| 6661 | ctgggctgct gctgcacctc cagcctctca agatgactta ctctagactt aaacaagcct |
| 6721 | ccttatttgt aatagaggca cctgattgag ctatgggggc tggttgtcca gaacttcgtt |
| 6781 | ttgtattaat tataccatat tgaaataaaa atctcagggc tgttggtaat cctttatgga |
| 6841 | atttgtgtag gatgcttatc agatatctcc ttactagtgt gcctgccctc tctaccttgg |
| 6901 | tgtatcactt aggaaaggag cagaaagctt gttgctgagt ggacatgagc tctggtcgaa |
| 6961 | tgagcttggc ccagttgttt ggcctgaaat ctaacttggt gaagaggagg ctcattactc |
| 7021 | tggtgtccag agggggtact cccttacaga tgccatgccc tcctgccctg gacacacatt |
| 7081 | ttgccctgtc taccctctgc tctagctagt gctctagtct cctaagaagt accttccatc |
| 7141 | aatctcattc accaccacct tcctcccaca gacagttgga gaaataggga cctgagtctg |
| 7201 | aatcttatat tacattgaac tataattata tgacaaatga taaataaccc aacaaggatg |
| 7261 | ttttcattaa agagatggag tctggcaggg atggaaactt gtgagggcat ctgttattca |
| 7321 | gaacttctca tttagtaaaa tggtgatgag caaagcaccc ctagccctgc agtctcccag |
| 7381 | catagaacca gttgtcctga taacatttgt taatgagata agatgtcttt gtccccctgc |
| 7441 | accaggtccc atccagtgcc tacagggcca caccctgcat cccctgtgtt tccactcaaa |
| 7501 | tttattttct ttcagctctg cagctgccct ggtgcactca tctagtgcct tttttaccaa |
| 7561 | aacttgctcc aggcagcaga tctaaaagat cagtttactt accccactat catttactct |
| 7621 | aatgtggatt aaagtttggg ctgtctttcc catagggatt tacatttgga gattccggtt |
| 7681 | tggtgaggaa attaccagag agctattaga cacttgggtg ctgttctcag ctttgtgatt |
| 7741 | aacacacttg gacatgttgt ttggcctctt tgggcctctg tttactctaa aatgagagcg |
| 7801 | ttgaatgaga tcagtgactt tcaaactttt cgttcctata gtaagatcta tgttttacat |
| 7861 | gactacccag tatgtataaa catacacaca caggttatct cagaaactga caatgtgctc |
| 7921 | ccttattttg ggtaaaacac ttttcattaa cagaagtgtt ctggctttga cccatgaatc |
| 7981 | gatttcatta cttgttaatg tttgaaagac attaaaagat gaatattaag attcccacac |
| 8041 | tcagatattt tgtggtttgt tttcggggaa tgagggaatg ccatttgctg ttttgtgaaa |
| 8101 | tcgttccacc aagggtcagt agtgattcct ccaagtttcc ttgctgtgcg agtggccctc |
| 8161 | taccaagcat gttcttccac cccctctcta ctctcgtcac ccgctgctgc cctcatcact |
| 8221 | cagtgcactg cactgggagc cccgacattt tccaacgcgg gaaagagagg gttgggcagg |
| 8281 | gggaggtctt tggccttggc ctcctcggac tgcctgggtg tgctccttct cattggcgct |
| 8341 | gtaaaatgat gatgtaatag tgcctataac acagtctggc acacagtaag cactcaataa |
| 8401 | atgttagatg tcattgttat gttctgccag aacaccattg ttggcattca ctttaacaaa |
| 8461 | aaaaaaagct tatgttatgg atagacacgt tgtatcaaaa agtaacattt gtttcaatgg |
| 8521 | aaaaagaggt tagaagctag agtttcaaac atggcagagg ttgttttttc cccacaagca |
| 8581 | attcaacagg aatgggtggc atctgagtgt agctatagca tctaaacatc atggagcagc |
| 8641 | cccagcacca gattccaagt cgtttctgct gagggtaata tcacaattca tccttttcct |
| 8701 | gggcagcgca ttgtcactac ttcagccact atttcttcag tctctactcc tcttgactgc |
| 8761 | ccagccaccc tccacccctc cttcccagcc agcaaccttc ccttcacaga gaaaatggac |
| 8821 | atctcttggt ttcctaagaa attcccggac aaaacccatg cccagctcag ctgtcctttg |
| 8881 | agtcttacct cttttgccct tcagttatct ttttccttct gggcacctag ctgctctcct |
| 8941 | ttgggtcctt ctcaagcttg ggatgttttt cccatcttaa agcaagtagg aagattctct |
| 9001 | tgatcgtctc tttagctact gggtatctct tgctttcctt tttattttta tttttatttt |
| 9061 | tttaaattgt tcttgccttt tagtgccaca cctgcagcaa gggaagttcc caggctaggg |
| 9121 | gtctaattgg aactgcagct gctggcctac accacagctt atggcaacac cagatcccca |
| 9181 | ccgactgaac gaggccaggg attgaaccca catcctcatg gatgctagtt ggatttgttt |
| 9241 | ctgctgtgcc acaacgggaa cttctctttt tccttttatg ttcaaacttc ctgaagtgtc |
| 9301 | tgactgtaac ttcctgctga cttctcttac acctcagcct cctcagtcca attcctgaac |
| 9361 | tgctccccaa atcccaatag cctttattgt cttttacatg actccttagc agcccttgac |
| 9421 | cttcctaccc ttgaccttca tgaggtgtgt tccctagtac tgctcccttt ctctggtgag |
| 9481 | ctctcctaca gccttggcaa ctcctcagtt tccggggtcc ttttgctctc cctcctgcag |
| 9541 | ctgtgaagta ttggactcgg tcaagcgcag tctctgaatg ctctcttctg tattccctcc |
| 9601 | ctacagactc ttcttcttcc tcactgacag ctgcaggtgc tatgtgctgc ctggggtctg |
| 9661 | atgtttacac ctccagcctg acctcttggc taagcccagg atgcacctgt ctcaacccta |
| 9721 | atgtccccag agtcggcatg tccaaagggc tgcacacagc ccccttgaag ccttgcttca |
| 9781 | gacttcaacc agaatgatct taccacaatg tgagtgtaat tgtgtcgctt tgtgcttaaa |
| 9841 | atacaccatt gtccttagga taaagtccag aagccttaat aagaacaact aggatttttc |
| 9901 | atggactgcc tctttttttc ctcaccccct tcccactgca ctggcctcac tcaccagcat |
| 9961 | tgatgctctc gaatccattc tatgcttgaa ctgccaccat catgtcctta acatctaatc |
| 10021 | atctttacca tctccttcat cctccacccc tcctctagct ctaggtctca tttttttatt |
| 10081 | ctaaaacact tatagacatt tccttcaaag agcttcagtt tgtaattaga tattcctgca |
| 10141 | tgtgagtgtt ggaaatagca ttgctgtccc cacgtagccc ataggctgca tgattgaggg |
| 10201 | ggcctatttg ggctcagcac tgcacttggc atgtgtcggg cattgtagaa gtattggttg |
| 10261 | agtgaaggag ccatggtctt tgcacgttct tgtcattttc atcacccatg gtctggtgag |
| 10321 | ccaggcacaa agccactgaa acaaagcatc cactgaggtt tggtgtcctc agctttccct |
| 10381 | tgggtcggtg tgagctcccg catcctgatt cacactttct cagcccagtg gttttcacat |
| 10441 | ttttctgggt tttatatccc atgtcagtaa aaatgtgaac ctgagcccca gccctaatat |
| 10501 | gtctaccttt tagttacagg tgactcacat acacttcatg taaatatgca tattgtgaaa |
| 10561 | cataacaaaa ttaaaaagat aagttaaaaa atattaaacg tctgagtatt tttttcctgc |
| 10621 | accagtctat tgtctggggg ccatccactt gggagaccac actcctccag tcaattgcct |
| 10681 | gtgttatttt taatttggtt atgaaagttc atgccatgta agaaatgcct gggccttatc |
| 10741 | aagctctcct tccttattaa gatcttccca accctggtgt tgatgggtag gagtggagcg |
| 10801 | cattcctcac ccttctttga taagcaggga aaatccgggt ctgcccgaga ctcccagcaa |
| 10861 | atcaagtgtc cgctgctgcc tgtcagctgg gtgctgcctg gtcaggctgt gtgcacagac |
| 10921 | acacccaagc tccctcaggt cgtctcttaa gatccagtaa ggctagcccc tgggctactc |
| 10981 | ctgcagatgc ttggaaaggc ccagaagtaa tcaggtgaga ggatcccatg ggccgcggac |
| 11041 | ttgggcgagt cagggaaggg ggaggaggtg ctctcctatt atcctgcagg ctggaaatct |
| 11101 | gtcagaggca ggatctgtgc aggctagaag ggcaggagtt gatcaccaca gggaaacctg |
| 11161 | gagaagacag gcccgccggt gtttctatgg gggcacttca gaaagttgtg ctgagtctct |
| 11221 | acagtcagaa gtggccagaa gctctggatc ggtggattct gctacctttt gccaagggca |
| 11281 | tgagtgagag agtgagtgag ctgggagtgg attgctgggg gagagactca ttctacataa |
| 11341 | ccaactttcc cctgagctat tgcctcacca acaggccggg tgctggggct gtggtgggag |
| 11401 | ggtgagggga gcccactttc acctgtgagc acaacattag gaatgttctg taagtatccg |
| 11461 | tgtagcaggt agaatttatt gaatctcatt atctgtgctc tgggctgggg gaggtggtac |
| 11521 | gcatatgggg ggagtgggtt acagaaagct ggcatttcca gtttctgtag cagagttggg |
| 11581 | gaggtatagc taacacggca cttaggcagg aggaaaggtg tgtggttggt aaggcagatt |
| 11641 | tcaaccccag ctgtgcatca gaacccacca ggggagcatt ttaagaaatt cccaggctct |
| 11701 | acccaaccac caagtcagtc tcccaaggtg gtgcccagtc tctttttaga atgctctaaa |
| 11761 | gtgagctcta aagcatggct gggtgccgcc acattgagtc agggtgcaga acttcacagc |
| 11821 | aggtaggaag aagtaaacca aatggatgta agagagagga aaatggagca aggaaaacag |
| 11881 | caaagaggaa agcaggtgca ggatttaacc tgctggggtg ctgggacccg gttcttagcc |
| 11941 | tgcccatccc tgccctgccc tggccacaca gatttcacat gtgcccgcat tcctgctcat |
| 12001 | cacatttgtg attttttgtt tactggtatc tcatgagtgc taagcgggat ttattgagta |
| 12061 | aggctggatt tgatccatcc ttggtcttca gggaactgag aggctagtgg atattttgta |
| 12121 | aaaaggaggg aacctaaggg ccatgcagtc aggggtattg ggctgtgcca ggaccccctg |
| 12181 | agccccatca cttgaggata tctggcaggt cacctttttg aacctcagct gcctgacctg |
| 12241 | taaagtggtg actaacacta atacctgttt cccaggggct gttgtcagga tggagagaaa |
| 12301 | taaggtttat cccaggtatg gactgtggac agcccctgaa gcttagtaga ttctgaccag |
| 12361 | cctaggcttg attgagctgt tggaatggtg tagacagccc tactgaggaa ggcccttgct |
| 12421 | cactatctga gcttgtgtgt ggcgggagta cttaggaggg ctcacttgga gctgctcagg |
| 12481 | tgggtgggcg agggcaagaa gggccccagg cttcttgttg aggtggagct ggagcaacat |
| 12541 | cagccaagca cagaggtccc ctgctctagc acccctgccc atcaggagtt aggggctgtc |
| 12601 | tgcctgtctt gctctgtacc agtagtacca cttgcctctc aactccctcc tggctgcaga |
| 12661 | actctaggtc tgcccctctc aagcctttgt ctttcatcca aatggctcct gagaagtttc |
| 12721 | ccccaggaag tcttccttga ctcactagaa taatgggaac aaattatgtc catcccaggt |
| 12781 | gatgtcaaga tgtaagatcc tgggacttcc gggtcacaca gtagtgttct ctctcctgac |
| 12841 | actggggtca cctccttgga gtgtgcatca tggcccctct ttcccttggc ttggtcagtg |
| 12901 | tctctttaat cccctcgcca gagggcctcg ctgagtgttc ccagagccca ggacagctcc |
| 12961 | cctgcagctg ggtgcatcag gaaggctctg taatgatgtc atgtggcctc cagcctcccg |
| 13021 | cctggcggcc agtgggaagg atctgtctgt tctggcacag gggccgcttc catatggagg |
| 13081 | gccaggaact ggccatcaca gtcaccgggg cgttccttcc attgctgcat gtttcccctc |
| 13141 | ccctctggaa gctctcaggc gtacccctgt gcaacccagg gagagtgggc aggcgtggac |
| 13201 | tggagtaaag ctccccagct gtctgggagg cctgaagaaa cctctgcaga ggttggcctg |
| 13261 | ctgccataat gagtggttct cctctgagct cccatctagc ttgtggctct gttgcagaca |
| 13321 | agcctttgtg tgtcttggat gagagagctg gtgttttttt acgctgaggt atggtttctg |
| 13381 | agtctgggga ggcttctagt gtagggccct ctgaggcctc tgtttggggt gaggtacagc |
| 13441 | agtaggcatg ggagaagaaa tggaatgttg gcttctcaaa agtgaggggt acttggttca |
| 13501 | gatacttggc ctttgtagat gttcaggtat ctttccctag ttccagtggc cagtgtggtc |
| 13561 | attgtcagaa gtggccaccc atgtgggtca gatgtgggaa tggaaaagca cctgtgaggt |
| 13621 | tgatgcatgg ccttagattc cagggaggtc tggatcctgc tttgaggtgt ccactatgcc |
| 13681 | caaaaccaga cttatattga gtgcatgcct tgctagcccc aggagtctta aaatggccac |
| 13741 | agatgtccct gatgagcagc agtgactggc tagggcctga ggttaaccag agttaggagg |
| 13801 | gtgatttctg cagatagaaa atactgcgga ggatcattca tttttacacg ttgattgaat |
| 13861 | gcctgccacg tggtgagttg tgttctgagc attctggata tagcagtgaa caggagaagg |
| 13921 | tcattatttg ggctgcccct gtggggcttc cattccatca agggaaacca ttaataagtc |
| 13981 | aatcaaaggc atgactgctg gtgtgatagg tctcatgaaa cataaagctg tgttaaaagg |
| 14041 | gactaggtgg tccaggaagg cttctctgag gaggtggcat ttgagcaaag actcgagtaa |
| 14101 | cctacactag acttctggca cccatgagcc aggacctgta gaggtaccta ggaactctct |
| 14161 | tgctgaggag caagctgagg cccagaaagg ataagacttg aattcatgag ctgattaatt |
| 14221 | ggaaagttga cctgaaacaa aagagtgaca aactgtaatg ggccagtact ccctgttttc |
| 14281 | aaccttaggc gtgtccccag gccaactgac cctctgcctt ttcgtcccag aatgtccttt |
| 14341 | attatcctct tgagacttag ctttttaaag atttgtactt ccaaattgtg tttattaaca |
| 14401 | atggtaggtt taccttctca tttgtggctg agtacgttga ggagagtggg gagcagaaac |
| 14461 | acatctgtaa ccgccagtca tccccccagg caagggctca gcacctggaa ctggccttgt |
| 14521 | tcctgtctca gcagagatgt ggcccagttt gggtaaccac aggctctcgc tgcctggcac |
| 14581 | agtgggattt ccatctgtcc ttctgaaata gtttcttgac tgctactttt tgggttccat |
| 14641 | ttggatcctt caggccctct cacctggttt gaggggccag atgctttggg tgagcaaagg |
| 14701 | agatcctgct gaaacctctg atgaagggtg gtgaggccgg gcttggaggc cctagggctt |
| 14761 | attttaaggg gagaaatcca cagtcaggtc acaaatccag ccatgaccca cctctcccaa |
| 14821 | ggaccgggat gagccttaat tggctgagag ttttcatcta aaagcaggca tcttgcatga |
| 14881 | atatgaaagg agcttctgat gaagtaagac ttcattaatc agcctgttag gaatctggct |
| 14941 | tcccagtatt atgggggctg gataatttga tgcttactgt tgtgctttca gcttataaag |
| 15001 | ggaaggcact agattttcat ctttctgcta aatattgaga gaaaatagta tctttatgtg |
| 15061 | ctttgtttgt ttttaactgt tgagtatctg ggtgccagac accatattta ttcttactta |
| 15121 | atctgtatag aaagactaca gaatgaatgg gattattccc actttcgtag atgaggctca |
| 15181 | gaaagctagt gaagtgtcta gctcaaggtc atatagtact gtggagcagt gctgggtaca |
| 15241 | aagccaggcc actggcttca caccttaact ttgacctctt acctcctccc agccacttta |
| 15301 | gggctgctgc taatatggaa ataataactt cagactccta actttcattt ctttctcttc |
| 15361 | tctgttaaaa tctgctgcat ttgttccccc aagaaagtga aacagatgat actagaactt |
| 15421 | gtgactttta aaactgtttt attgtgaaat agaatacatt gtaaatgtga ggttcagtga |
| 15481 | cttttctcct ttagattttt tttccccttt aaatgattct tttacactat cagggaaatc |
| 15541 | ccagcaaaat caactttcca tatgcctgtc ctgacctttc tgtgccaggg tcttaatata |
| 15601 | tgtgtgtctg ggtttccact gacagctcta atatacttca aacatgaggg cttgtgcatg |
| 15661 | ccaatgagaa agtacttagg atacctcaaa gtgtgaaaga gggtctggaa agtgggagga |
| 15721 | agtccaaatc gtcctccaat aaaagttgcc taagacaact tggggcttag gtgctacttc |
| 15781 | aaaaactaac aatgcattcc acctttccca aggggaagtg ttgtgaatgg gcatggcagt |
| 15841 | gccgtgtttc ttgccctttg ttgagcacct gctccaagcc ttgcttcatg ctaggttttt |
| 15901 | ggcaagcatc acatttgaat cctttagaat gcccctagga tacaggtgtg accactccgt |
| 15961 | taccagatag agacacaggt ccacagaggt cagatggcct tcaggacaca ctgaacttgc |
| 16021 | ccctgtgtgc tgtagcagtc ccttctgctt gtccatccca gactggacac tttgggggct |
| 16081 | gaaggcaagg atacctttat tgttggagcc tggcgcagtg cctggcattc ataggttcta |
| 16141 | agtcatagca ttgaatggac ggaggtgagt taacttgtcc acataggtat agtggtgagc |
| 16201 | acattagatt gtggaactcc aggtctctat gactgttgcc cagcaggtca gaagtggggt |
| 16261 | ctgattctag ctctttggtt gaatgcaaaa tggcatatgt gctggcttat ggacattgca |
| 16321 | gcattgttat agcagaagac tggaaataac ccaaatactc atcaatcagg ggctagttaa |
| 16381 | gaaaaatggt ggtgcatgta caccatgaaa cagtgcaaat tagaaacaag gaaggttttt |
| 16441 | atgtactata tattacacaa acttcatgaa aaaaaaggta aagaacaata tataatatac |
| 16501 | taatttggga taaaagagaa aataagattc taaatttgct tgtgtatgtg taaacaaact |
| 16561 | ctgggaggat ttttaatggg tgggatagtt aactggatag ggtgggagag agaattaact |
| 16621 | gtatgttttc ttaagttttt tggttttgaa accatgagaa tgactgtatt gcttattcaa |
| 16681 | aaagttaaaa agataaaaat aatggaggtg accttggact agctattctc tgtatctggg |
| 16741 | cttcagtgga ttagatcagg ccttcatctg gagtggcatg ttgtgtatct attgtgtata |
| 16801 | ttgtgtctct ctggctttca tgagctcttc cggaagctgc tgcccctgtc ctaggatggt |
| 16861 | ctgtagtccc ttcttccatt ggtggcctgc acctgaatca tcagctcaga gtgtttgaga |
| 16921 | aagactgggg tgacccagag agccagaggc cttctgcaga ggtgtcaggc cttgttcctg |
| 16981 | tctccaggcc tgggacttta tcctctgaac accctgccct gatgtgactc aggtggttct |
| 17041 | tcccctaaag tgaaggatta cagagactgt tctggagcat tctggcaaag tgtgctgcag |
| 17101 | tttaatggga agaaagaaat ggcagaatgt tttcccaggt tatttttaat gcttttcagt |
| 17161 | tcagcaaaca tttactgcga actaagtgca aggcagtgtg ggggactctg agctacataa |
| 17221 | cacctcctct gctctctggg gcgccaagtt tcgggtaaga aacttgttcg taaaattcct |
| 17281 | ccctgtgccc tccaggacag cacggtttgc ctctagtccc gcagagtttt gtgggattgg |
| 17341 | taggaatcct tctccccatt ttacagatga ggaaagtaag tctcagtgaa gctttgccca |
| 17401 | aagctgcagt tcctaagtgt taaaacctgg aaccgacaaa aaaagacaca cacaaaaaaa |
| 17461 | cctggaacca aaccacactt tctgatggtg tctcgtgtgc ccagacaccc atgagtaaaa |
| 17521 | ctttctgcca aaggatattc cccaggggtg caggcaaggc ctgtttctac ctggaaacat |
| 17581 | tgggtgagca cagtcctgaa taaagctcct tttttccttt aaggaagggc aagagaaatc |
| 17641 | acaggcttat gcctgggtct tggatctgaa cccctttgct gtggtccaaa cagttctgac |
| 17701 | ttccactgct tgtgaagccc atcactctgg caccaggtac atgattctag aacatcccat |
| 17761 | aggtgtgtac aagtgcgtgt gacagacagt gggcttctcc ccatctcatt ctcacattat |
| 17821 | cactctgttt ccttcccttc cacaaattcc gtcaccagta cccatcgtcc tggctgatcc |
| 17881 | ccaaaaggac caagcatgct gcctgctcag ggtctttgcc ctcggtgttc cctctgccct |
| 17941 | cttcacttgg cttcaagtcc tcactgaaag ctcagcttag aaaggccttc cccaggatct |
| 18001 | tttgtgaagt actgtctttt gtcacgattt cattttttcc cctctgtccc tttgttgcat |
| 18061 | ttatatgtat cgtacataga tatctatact tactggctta ttatctcttc cccactagat |
| 18121 | tgtaaaccca cgggttaggt gcctgttttt gttcattgct gtttctgttt cctagcacag |
| 18181 | gtgcccagct tttgacagat actctgtaaa cattgttgaa tgaataaatc aggcctgtgg |
| 18241 | cctaaacaca taatgcttct ttaaagaggg aaagcatcaa atcatagcat tttaagagga |
| 18301 | ggaaagatcc aaggctcatc tctttgcagg gcttcatcat acaatattat taattcattt |
| 18361 | aataattgtt cagtactgtt catgtggaaa gttctttgca gaagtcactg agcagcctct |
| 18421 | gatgcataca cttacaggga caggggctca ctgatttgtc agagcagccc attctttggt |
| 18481 | gggacaagct gcaaagattt gaacaccctc ttatgtttta tgttggtcca aaaactgcct |
| 18541 | atttgtaatt cctgcacact gaccttagct cttgagctgc tggagaacac agaataaatg |
| 18601 | tactctgttt gcatacagct ctccttccaa tagtcggaga aagatagcta tcatgagttc |
| 18661 | ccccaccctc cagcctaaac atcactagtt ccttccattg ttcttccagt gttacggttt |
| 18721 | gcagatgcct tcctaccctt ggtgcctgcc tcaagtttgt tctgcttcgg gtgtcagaat |
| 18781 | ttaaggtctg gacccatcag aacagtgttt tgggaccctg accgaggacg cttggtccat |
| 18841 | ctcacttgtt tctgtccaca cagcctgtgt tggccatctt ggcagggatc tccttcatga |
| 18901 | gcttttgatg aaggctaact aaaatctcta ggctcttttt ttacattatg tctattcgtc |
| 18961 | tagcccctcc ctgactcagc aagaatcgta aaggtttggg ggttttgttt tgtctttgtt |
| 19021 | tttagcatct tgagattaaa gcagctgtag atctgtgaac agcaaagaat gcaacccgtg |
| 19081 | ctcgatttca caaaccagta aatataggta aagttctctc accaactctg gaagtacaga |
| 19141 | aaaacccact gagcctctaa ctttcttgct tctgccctct ttttgatgcc tgtgtccttt |
| 19201 | tcacagagtc tttctgtagc ttactctccc tcggtgttcc gtccctctcc cctccttcct |
| 19261 | tcctggctct cattccattg ccccgtttct cccttccagt tagtagtgta tggaattaac |
| 19321 | attactcctt agcatttcct gcacttccat ccccagtact tttaccttca tttccagact |
| 19381 | atggcaagct gcacatcaca tttctctaag gcagaaagag atgaaattgc ttcttatgtg |
| 19441 | tgacccttgg ctaatttgtt ttgtggtctt ggatagagaa aaaaaactta cagccctcag |
| 19501 | ccttaggttc ttttgcatga ttggttcaca tttctgccca aatcagcggt ttccagttgg |
| 19561 | tggttggaca caagggcctc agcaccgcgg tgtttgctct gcttctgtca ttgccaatgc |
| 19621 | tgctgtcatt ttggtagagg tggcagggaa ggaggccaag gtcgagggtt tcttctctga |
| 19681 | gccacttagc ctttccccgc ctcttctgcc ctcccagccc tcctgggcag tgtggggagg |
| 19741 | caggcaggaa ggcgggaggg ctggggtcag gcccggggct cttttcctaa cagctgtgtt |
| 19801 | ctttttcctc ttttgtttcc tcttgcttca aggagttcac cgtggagagg cctgtcatgc |
| 19861 | tggggtgtag accccaagtt aggaggatta agactggccc caggttaggt tctacattgg |
| 19921 | gctaaactgt gtgaccttgg gtgaggagct ggattgaatc cagtggttcc ccagaggtgg |
| 19981 | cgatgatggt gtcctttagt ttctgaagca catttctata gccagctgga aattcggcag |
| 20041 | aggcccgtac tagcctgcca gaagtgtcct gtcctgaatc tgaatggcct ggtgtctttt |
| 20101 | taaatgggct atgttgctag gctgggaagg ggctgtgatc tgctggctca tgatcgctac |
| 20161 | atggtgatgc cacggaaatt aattaacttt tgttgtgcaa accattcttc cacttggtcc |
| 20221 | atggggagtg gatggctgtc tccatccaca ccagacccac cacagaaact ctgagctatg |
| 20281 | tccccttttc acaccttgct gactcgactc cggtaggatt tccacaagtg gtcccagccc |
| 20341 | agctgggctt aaatccctga aattaatttc aggagtcaaa atcagccaaa aatcaggcag |
| 20401 | ctgagacaga gggccaggaa gccattttcg catctttttg tgtattttcc caagagtaga |
| 20461 | gttggggtgc tttgcttcaa aaatgaataa ggggtcagag ccattgctct ccccaactcc |
| 20521 | atcctttctt ctagggtgag tgagctggtt cagtttgacc acttagccag cccctctggg |
| 20581 | cagggcacca gaaggggata ccaagatctc agaccttacc aaggccgtgt ctgggggaga |
| 20641 | gactgtgagg aaagactgac aggtgtcttg tggccaagca gcagctctct gggtgccctg |
| 20701 | gtgggggaca gggtgtgttc cctggggagg gatgggagga gtggaattcc acttggcttt |
| 20761 | gggttcaagg aagtgaggcc tggggaagca cttcataaac gattaacaca gggaggaaat |
| 20821 | gccaggtggt gtgaagtgtg tgctgccagt cacgggcaaa ggggtggtgc aggaacaggc |
| 20881 | ttgaaaagag acatggggat ttagtcaggg ggcattgatg gtcagggggc tgggagtcct |
| 20941 | cctctggatt tggcgcctga tccccatgcc gtttaaaggc ttttggacag agagcgacta |
| 21001 | gacatgatgt gatgtgatcg aagcagtgtt ttaggaaggt ggcccaggat gtaccctctc |
| 21061 | cgctctctgg gccttcctca tcaagctcct tggggctctt ctgttgtgtg gggccaatgc |
| 21121 | ggtggctcct agatcatcgt gggtggtgtg ggtcttttta tgaaccacag gatcatagag |
| 21181 | cagtcattag gaccccatga gtccacgtgc ctactccctg ctgtcctgta gggtgatagg |
| 21241 | catcacatgg ggcaaggcca gatgcccata tttgaaacca gcaccttgag cacagggggc |
| 21301 | tgtttttatg atcctggtgt gttttaagag actacatgat tgactttcgc atttcggtaa |
| 21361 | ttatccggga taattgccca gcgtcctaga tttcctttct agcatccttc aaccatgact |
| 21421 | cctgggctcc tctggcctct gaagtccccc accttctgtt gaggaacata actgagtttg |
| 21481 | gatgtgggac gggactaaaa gtggatgggc ccagtgaatt ttggaactaa aagggatctc |
| 21541 | gtgtctgtgc tccattgttt agtgtgtgga ttagaggact gcagaagccc agagagtgga |
| 21601 | tgttacttcc cccagggcac actgcaagtc agtggcaaag tggggcccag agtccaggtc |
| 21661 | gttagactgc ctgatgagca tcctttgcac cacacctcca tgtttcttgt ccccctttcc |
| 21721 | ccggccaccg ttcagacacc acctgctggt aactgtgcct tcactggact ctgcttccac |
| 21781 | ctcctgcttc ttcacgcctt ccattacctg ctgaaggtag cacaggacag ccacttagaa |
| 21841 | gcagcctgct tcacttgatc atgtcattca tgcaccagtt ccagaaccct catctcttcc |
| 21901 | tgtccccgtt gcacttcctt tcccccagac agccttctgg gcctattctc tgctgctctg |
| 21961 | ggaatctttt acctgctctt aaatcaggca gggctagggc aggagctggg caatcaggga |
| 22021 | aggcttcctg gaggtggaac tgccaagtgt ctgaagtaga atagaatcta gactggctgg |
| 22081 | aagagaggaa cgaggtttga ggggaggaga gcaaaggcat gacatgaggg aggggcccaa |
| 22141 | gctctgccag gctcactggc taaacaggcc catctgacaa gcccaaagcc caggtccgac |
| 22201 | agcaattgca ccagttcttg agttactgag tctgttctac tggccagatg aagcatctgc |
| 22261 | agtggaacca gctcctctgt cttggtgagg cagaaaccaa gtcctcctca agcttggggg |
| 22321 | aggaggtagg aagccggttc caggcagcga gggagccagt cacagggttt atgttaatac |
| 22381 | gggccctccc ccagccggcg cttagcctat tacccgggcc ctgccgacag cttaattacc |
| 22441 | gctgcagcga gcctcggcct gccctccacg gttccctaaa ataataagga tggcgtcacc |
| 22501 | gacttcccgc ggccatgtgc tcccacccgt gccttcatag ctctattgtg ggaaacaaac |
| 22561 | aaaatccaga gatccagatg cttttttctt ggagattttt tttggggagc agggttaaag |
| 22621 | taaggtggga ccctgccccg tagttaagat ttcaaaagat ctctaatagg tatggagacc |
| 22681 | tgagtcttag ctccactgag gttgcagaat aacctcctgt tagcagaaac gaaaaagctg |
| 22741 | tgtggttgat tctgtggtcc tccaccgggg actgtgattt ctgattagag cagtggtgtg |
| 22801 | gtgctgtgca gtgtggtgta gcagagaatc acctatctcg gggttcctgg gctctaggcc |
| 22861 | aactctggac cttgtttctt cctctagcaa aagagagctg aactagacca gcaatttgca |
| 22921 | cactgtgggt cacacttgtt aaaaaatcat cttttttaat gtaatagaaa aaagtaattg |
| 22981 | taacagggct aagttttgtt tcagtgtgta tgcctgttta catgcaaaaa tgtgcttact |
| 23041 | gagccaaggt gtaaatgtat ttattttttt gactgtgggc tatggtcaga gagccactga |
| 23101 | cctagatgat cccgaaggcc tttctagctt tcataatgtc cagattttct cccagacttg |
| 23161 | ttccctatgg aacctgacag gatttttcag ttttgccaca ctgactccta agcaaaacta |
| 23221 | gagattgtat ggttaaaact acaaaaccac aagcatctat ccctcctatc gaacacctgg |
| 23281 | gaggaagccc atctccccct gcattggtgt ctcagacagt ttggccttca agctagaaat |
| 23341 | gggatctgca acaggggatg gcccatttaa aaactgacct atttaaaacc catgagcact |
| 23401 | gaaaaaacac ggggtcaatc tgatttaagt tgtcttcttt tgcgaagctg ggacctgaaa |
| 23461 | gctaaattgg tcggggactg acaaggttga aaccaaagtt acaaatattt attgcattct |
| 23521 | tgttcagtga aagaaaagat caagcctcaa gtttagtgga aaaaactaca caaaaggagg |
| 23581 | tgtgggtgta aagtccagca gggtgcattc tgggcacacc atcttcactc tccagaatgt |
| 23641 | ggagaggcct tcattccatg aagaagctct gcagacagcg gaggtgactc agagctgtgg |
| 23701 | gcctgccttg gttcccattc ttgtgccttg actgcagacg cagcagtgaa ggtggctgtg |
| 23761 | cattgagaag gcaggtggct ttttccaagg tgggggtgct ctaagtgcac tgtttaattt |
| 23821 | aatccaaccc caggtgatgg gcactatggt cattggagga agcggaagct tcagaggtga |
| 23881 | gatgagcttg cagtggggaa tttgggcttg agccagatct gtctgactcc agagcctgcc |
| 23941 | ttctgtccac ctctccacct tgcctcagca agtgctggaa aaataaaaag cagtcttctc |
| 24001 | ttcagtttcc ttctctctgg tgctggtctt ccgagggctc tgctttaggc ccatgctgtc |
| 24061 | catttaaatc ggggctagcg tcacgggcat gtgacctgtg cagtcacaca ggcccacacc |
| 24121 | tagcgggtcc ctccctgctc ttgggtttaa catgctactg tccctgtctt ggaattcttt |
| 24181 | atcgtccttg agcaggtggt ctcacatttg cattttgcac tgggccctgc aagttacctg |
| 24241 | tccaactctg gctttgctgt aaggcacccc ttttcttggt ctttctgggg ccctcctatc |
| 24301 | tgcccggaac gttcccatcc tccccaggta gaggcatgtg aagcacaccc cccaccccag |
| 24361 | ccttgggcct gctatctggc acactggact gtagttgttt accagctgtc tgtctctttc |
| 24421 | ttgcctgcca ccaccccact tgctggcaag gaccccaact gtggctcacc gtcacctctg |
| 24481 | tgtctcccct cagctgctca gatggacagt gtcacaggag ctcactgtgt gaagagaggg |
| 24541 | gtttctcaca cacgagacct gtgtgcagag agcactgggt caggtcaaga ggaggacgca |
| 24601 | ctgctgacct gcctccttat gggattatgc ttgtctcagg gacacagaac acaccagaag |
| 24661 | tagtagttgt ttttgtttgt aaaaaaggga gggcttgttc aacctgtttt ggaccagttg |
| 24721 | cactgaaagt ttcactggtg gtttgtctcc ctctgtatgc tgggcaccta atgttgtggc |
| 24781 | tggcacaata aagaggacat ttgttaagga gttgatgggt gtccactccc cgctaggctg |
| 24841 | attacagaga aggacagtcc ctgacctcta ggaggatcgg atgcttaata catgattaaa |
| 24901 | tgattcatgc agcagctgcc cttggtgccc cgggatgcgc agagctggct gcagaaggct |
| 24961 | ttctggggag ggggcttaac tagagctaaa ggcaggggtg agaggtgggg tgtgaagaga |
| 25021 | ttggtggaca tgagggcagg tggtcaagag tgaaagacct ccttaggtcc gtttgggacc |
| 25081 | ttgatgattt ttttcccccc cggaggcaac aggaagccag gaatgttttc taggccagag |
| 25141 | tttgcacttt ataggttaca cagataccat cgtccccttt ctgtgaggca gatcctgaga |
| 25201 | cctcggggag agagtcaggg tcattcagcc agccaggcct tggcctccag gtcaaggcct |
| 25261 | gttccctctg tcatggagac cccatttgga agcagaggca gacctagggt gtcttagatg |
| 25321 | accatgacca agactgcagt gccggcagca gagaggaatg gtcaaatttg agagatcagt |
| 25381 | aaagacgaga tttcccagtt ttgaaacaag cgacgggttg ttccttagag gatagtgtgt |
| 25441 | tggtgtttat cacggacagt tcagctcccc ttgatgagaa atggctgctg tgtccttgct |
| 25501 | ttggggacac gttttgcagg aaatgccaga gccaggcagt ctgatctctt gggggctgag |
| 25561 | tacgaagatg caggcacctc tcaggtgtcc acacttgaga aatgaagcat agggtcctgt |
| 25621 | gtaggtttgt tgttgaggtg tttgaaaagc tcccgacaga ggcagaagaa ctccagttgt |
| 25681 | cggactagag agagagtaac cgcgtttgga tatttttaaa cggccagtgg aattgggaga |
| 25741 | gagtctaaaa cccaaagtac aaattttcag tttaatttta actttgcact tagctcacac |
| 25801 | ccagggtttg gcagcgggga atgggcctta cttttctttt tttctttttt ttctttttta |
| 25861 | agcagaacac accctggatg taaaagggat gttctcttca catggaagag tttggggcgg |
| 25921 | cagcaccgag cagagagggg aggaagtggg ttggaattgc agcaggattt cagagtcaat |
| 25981 | ttaaggaaga ttggggtggt tggatgtttt gaaacacatg agcctggctt gggagctgta |
| 26041 | agagagcatg gccctgtgtg gaggcaggtg gagcaggggg tggacagcag acttccacag |
| 26101 | gtcagctgtt gcactggagt tttttttttt tttttttttt tcttctattt gatccattac |
| 26161 | agacgcttca caggctgctt acctgccatc tccaatgtga cacatcaccc caaaaagcag |
| 26221 | cagtgggttt cctccgatgt ggtgtgggct ttctggtgat atcagcatcg ttggggtgta |
| 26281 | agcctcagcc catctctccc ctccttgatc tcttcccaaa ctgcgccaaa accctgccag |
| 26341 | gaaggaagtg cagaaagcgt ggtcatcctg gaggccccac agacctggga gagccacccg |
| 26401 | gcttttcaga caccaaaggc tttccctccc aagtgatgag aggtggcctc ttgctccttg |
| 26461 | gggaggagca ggcagggtct caggccccct ggcccctctc cagaactgcc ctccctctca |
| 26521 | gcagcgtcat cagagcttgg gctgagctgg ttttgtgctt tggttactga ggaagtgtaa |
| 26581 | ccagggtggg gatggctggg agtggccccc acttggctag tggaacagtt agaagagggg |
| 26641 | ctgaaggatg tgtaggtaca gtcagggcag cttgttggat tgaattgggg gggtctggaa |
| 26701 | acaatgagga acccctcctt atcctgcaac acttactgcc gccgggagtc tgaacacacc |
| 26761 | ctcctatgcc cattcctggg actttaggaa cactgagccc aatgtggcta taaaatggga |
| 26821 | gaacacccca taatacccca tggagcagtc gtgaggattg gatgagatag tgcctgtgaa |
| 26881 | gagggtgcct gctgagggcc caggaaagat cctgtggtag ctgttactat aaacagtaat |
| 26941 | ggtggcttgc atgttgtccc cgaggggagt ggccaccact ttgggcttgg tgctgggagc |
| 27001 | tgggactgga acctgtgtct gtctatagga ttctcaggca gcagcatgcc tcttcaggaa |
| 27061 | agaggccgac atccgggagg aattcccttg actccgcagc accacctttc cttgttcctg |
| 27121 | aggccttctc cttggctgat ggccatagag aagctcttga cccgtctgga aaaaccacat |
| 27181 | gtctgaaatc tttgagggtg gactggaaac ccttctcata tttttctttt atgaagctta |
| 27241 | aagaattccc agggccttcc aaggaaagga gtgactgaag acgcctaggg cgcgtgactc |
| 27301 | cagcagtgac tgcgcacaga gggggtggct ccccctcctt agtacagttg atctctctcc |
| 27361 | acttgctctg ctcccctaag gcctggcttc ctggctctac actgatttcc tgccctaggg |
| 27421 | caaaggcctc acaggattta ttcccaggca ctcttgccag tgaccttgag aaggtggcct |
| 27481 | gacctggaca tctggcctct cctggggact gagagggtgg gaaaggacat aactacccca |
| 27541 | ttccagatcc aaggggaagg tggccctggc accagtgcct aggccaagag cttgctagct |
| 27601 | tcctggcaga ttgctgctgt cccaggctga gcgggttacc cctccaccag ggaagaaagc |
| 27661 | agggcagctg tggggccctg tggccagatg cgccctgatg ttccctggtc tggtcactgg |
| 27721 | taagggtaga ctgaggaggg catggcaggc ctgctgtgga ttctgttccc caagttggcc |
| 27781 | catggtcccg tgatgcccaa ggaggagggc atcttggcag acagtcttcc tgagcccttc |
| 27841 | aggaagaagc tggtcagacc agcctgctca gaggcaggga gcagccccag acagggactg |
| 27901 | gttagaatag gcctgtctac ttatccgtga ggagaaaagg ctatgagggg caggaatgac |
| 27961 | aatgctattg agaaggacag gcctgtgcca tgtgaccagg ggtgagaaat gcaaaccggt |
| 28021 | gggtatgtgg gcatcagtac atgtttccta gaggctgcaa ggagcaggca ggtggggata |
| 28081 | ctatgtcgag caccaggtgc caccaggagg gatcaccttt aaggtccctg caggccctgg |
| 28141 | aagcccaggg agcagccccc gatcagcttg gaacaggagt ggaaaactct ggtcatccac |
| 28201 | ctggagaagc gggcaccctg gcctcccttg ctgactcccc tgggtcattt caggcctgag |
| 28261 | acctgatgac tcttcctcag ccatctccac accacctgtg ctggagccag gacatgcctt |
| 28321 | cagccaggtc tgccttttaa cagcgaatga gacggtagct ctttatcagt tctttctacc |
| 28381 | tcattcgaaa taaaggaggt agggcccaga ttgtaggtaa gatgagaaga ggggaatcta |
| 28441 | agaactctta acagggcaaa atcacctcca gggtggtggc gggggtggga gtggggtcat |
| 28501 | ggcacgcgtg gccacagaag tgcctccaaa gaccaacttt tggactttgc cgacctgagc |
| 28561 | atttctctgc ggggccatcc tgggtttgtg gacgttcatt ctccttccac ctctactctt |
| 28621 | aggactctgc agagaaagtc tagtgctggg atattattcc atatggtagc ttgtatgtgt |
| 28681 | ctctttacac ttacattaaa taaatacagt gtaaaactta gctcctcagt ctcacctcag |
| 28741 | tgactgcatg tggctagtca ctcctccctt ggacaggaag ttctactggg cagtgctggt |
| 28801 | ctagagaaca gtgagtcaga gagggtccct ggtccgctgc ccttgctctg gggcaggcca |
| 28861 | gtacagtgac caggtgatga atgcctgatg gcaggggcag gtaagagcaa gcatgcgggt |
| 28921 | gggtctcggg acccatctga ccgcagaagg tcacagacac acaggtgaaa gggcagtggg |
| 28981 | ggctgaggat gggaagaagt aggtccctgg cttcaggaac atggccaggc cgaggccaca |
| 29041 | cggggcactg agatgagagg ctctgacaga atgacagggc ccagaggttg cctaacaacg |
| 29101 | tgatgtctgg catggtgggg agctgggggt tttattgttc aatggaattt ggaagaatgt |
| 29161 | ggcagttggg tcttgccgac agcagttggg tttatcgagt ttgctttctc cagtccagtc |
| 29221 | aggctcctgg cgggggagtg gggggacctc ctggtttgca gctctgctca cgccagggac |
| 29281 | ccccctcccc acagtgttgc tacaggcaga aagcctgggg tgggctctgg ctctgccatg |
| 29341 | ctctagaggt gccttctggt ctgcctcctg tcccctcccc ggtcttgggc cccattcctc |
| 29401 | accccattca cgccggcctt gtctttccag gtgaaagagc tcgtcctgga caattgtcgg |
| 29461 | tcaaatgaag gcaagattga aggcctcaca gatgaatttg aagaactgga gttcttgagt |
| 29521 | acaatcaatg taggcctcac ctcagtcgca aacttaccaa agttaaacaa acttaagaag |
| 29581 | gtaaatagag cgcaggaggg gctatgcccg ggaggcctgc cagttccccg agcctgtgtg |
| 29641 | cgggagccag gcccttgagc ccagggcctt cgttattgta agagctcttt ttgctttttc |
| 29701 | tctccctgcc ccgtttagct tgaactaagt gataacagaa tctcaggggg cctggaagta |
| 29761 | ttggcagaaa agtgtccgaa cctcacacat ctaaatttaa gtggcaacaa aattaaagac |
| 29821 | ctcagcacag tagagccact ggtgagtcac tggggttctc tcctctgtac tggggtggga |
| 29881 | gggattgatg ttggggaatg ctgcctttga tttgtataac atgctggcgg gtgggagggg |
| 29941 | atggcttatc tcactttatt tgactgtcat tatatgctgt gagatggagt tggagctcct |
| 30001 | acttctgctt tgtccatgaa gaagctgagg cctagagacg ttaaagagct tgaccaaggt |
| 30061 | caaacggtca gaacaggcat cagggtgaag gaaaccctaa gtcccaggtc tcggcttcca |
| 30121 | gtccagcact tttcctcttg agcgtttctg cccagccttg gaaacccgag ctgtcatttc |
| 30181 | ttcaggctta gagcacggct tgtgcccttc actggggagg ggggaaccac ccaggaatta |
| 30241 | agcttaccct ggaggtggcg acctgttttc tccatgggtc actagccaga ggctactgcc |
| 30301 | tctagattgt ggcaggtggt cctgacacag gcccctggga tgacagagag ctgtgtggca |
| 30361 | tgggagtccc atgtaggctt tcctctgtga gcacagagca gagtggctcc gggtcatcag |
| 30421 | tcctgagtgt gatggtgacc agggcctcag agccctgggc ggtttcttct tctgctcgtt |
| 30481 | cacatacagc tgagcatacc ctaaacttta tgattcacta gccagaaagg ggtaggtcat |
| 30541 | ggtcagaata gcaatttctc cattatcctg gtctgagtga atgtattctt ttgagttcaa |
| 30601 | ggaaatcaac tgtaaggtgt gtcattgatt aatgatagct tttttgaaat atagaagtac |
| 30661 | tactgcatta cattaacaca ttgtaaatat gaatttgttc ataagccatg caccttagaa |
| 30721 | tcagggaaag gccatgagga tatttggcca agttggccat gtttttcttt tctgggtaga |
| 30781 | tagggtggca gttggggcat ctctcagggt ttttgtgcaa aaagcattcc ttgccctcca |
| 30841 | cagaagctca gggttcaggt tcatgggcca gcatggcaga accggaagca gacatttctc |
| 30901 | ttccaaactc agactttacc tgcccttgac caggtctcag tcacagactc ttaagagagc |
| 30961 | aacctagttc ctgacactga ccataagaga cagtaacttt aatcaccagt caggcctggc |
| 31021 | cccgttggaa cagggacttg gcctggggca gggtacctgt gtcttgaggg gaatcaagat |
| 31081 | gtagctgttc cttgttagct gtgtgaccct ttcacttcct aggcctcaga ttccagccac |
| 31141 | caaacaggtg taaggtgtag tatgggctga aactaatcgg taaaaagctt tgcacaaagc |
| 31201 | aaatcatgtt aagaactact gacaaaatct gaagacttca tttcctcttg tcctttttga |
| 31261 | aagtcggtgt tggaagagta aaagcacagg tctgaggttg ggctgcctga tccaatccca |
| 31321 | attctctgct ctcttcttcc tcatctataa catgaactga taattatttc tgccttattg |
| 31381 | gggctataag gataaattga gttaataaag tgctatatta attgccgtta ttacttttat |
| 31441 | tcacagaagg agttagttat gttcctgtgc ccttctcagt tcccctctgg caccttagtc |
| 31501 | acaggagcag gcagcaaagc ttttggcacc aagagagccc ggtttatttc aagcccgcca |
| 31561 | ctgttacggc aaaagctgtc attgccaaac aaaagaacga ggtcttgcag atccttaagt |
| 31621 | ccaacccccc atcttccaga taaggaacat gaaccccccc ccctccgccg caaaccaggg |
| 31681 | gtctcatggc tgagcaaagc cagagtttgc ttgtcacctg cgggcgggaa gctctgcaca |
| 31741 | cctctcgtgt cacccccaac tgatccccct gccactgtcc actctgtttg cttgcagaaa |
| 31801 | aagttagaaa acctcaagag cttagacctt ttcaattgcg aggtgaccaa cctgAATGAC |
| 31861 | taccgagaaa acgtgttcaa gctccttcca cagctcacgt atctcgatgg ctacgaccgg |
| 31921 | gacgacaagg aggcctctga ctcagacgct gagggctacg tggagggcct ggacgacgac |
| 31981 | gaggaggatg aggatg |

Porcine ANP32B has seven isoforms, the complete nucleotide sequences of which can be found in the Ensembl database under Accession Numbers ENSSSCT00000005912.4, ENSSSCT00000054395.2, ENSSSCT00000068404.1, ENSSSCT00000062686.2, ENSSSCT00000045745.2, ENSSSCT00000044227.2, and ENSSSCT00000055524.2. Of these seven isoforms, the isoforms encoded by ENSSSCT00000005912.4, ENSSSCT00000054395.2, and ENSSSCT00000068404.1 align closely with human ANP32B. The complete nucleotide sequence for ENSSSCT00000005912.4 is provided herein as SEQ ID NO: 4. In addition, a partial genomic sequence for ENSSSCT00000005912.4. is provided as SEQ ID NO: 5. SEQ ID NO: 5 is used as a reference sequence herein. SEQ ID NO: 5 includes nucleotides 2 through 20,343 of SEQ ID NO: 4, corresponding to exon 1, intron 1, exon 2, intron 2, exon 3, intron 3, and exon 4. Table 3 provides the locations of the exons and introns of the ANP32B gene in SEQ ID NOs. 4 and 5.

TABLE 3
Locations of exons and introns of the
ANP32B gene in SEQ ID NOs. 4 and 5

| | Location in SEQ ID NO: | Location in SEQ ID NO: |
|---|---|---|
| | 4 (nucleotides) | 5 (nucleotides) |
| Exon 1 | 2-410* | 1-409* |
| Intron 1 | 411-10,823 | 410-10,822 |
| Exon 2 | 10,824-10,973 | 10,823-10,972 |
| Intron 2 | 10,974-14,272 | 10,973-14,271 |
| Exon 3 | 14,273-14,395 | 14,272-14,394 |
| Intron 3 | 14,396-20,153 | 14,395-20,152 |
| Exon 4 | 20,154-20,343** | 20,153-20,342** |
| Intron 4 | 20,344-22,651 | — |
| Exon 5 | 22,652-22,776 | — |
| Intron 5 | 22,777-24,438 | — |
| Exon 6 | 24,439-24,513 | — |
| Intron 6 | 24,514-25,741 | — |
| Exon 7 | 25,742-26,343 | — |

*Exon 1 of the ANP32B gene includes a 5′ untranslated region (5′-UTR) at nucleotides 2-356 of SEQ ID NO: 4 and nucleotides 1-355 of SEQ ID NO: 5. The start codon is at nucleotides 357-359 of SEQ ID NO: 4 and nucleotides 356-358 of SEQ ID NO: 5.
**Exon 4 of the ANP32B gene includes the nucleotides that encode asparagine 129 (N129) and aspartic acid 130 (D130). These amino acids are encoded by nucleotides 20,211-20,216 of SEQ ID NO: 4 and nucleotides 20,210-20,215 of SEQ ID NO: 5.

Table 4 provides an annotated version of SEQ ID NO: 5, showing the locations of exons 1, 2, 3, and 4. Exons 1, 2, 3, and 4 are shown in underlined text. Coding sequence is both underlined and bold, while untranslated sequence that is part of exon 1 is underlined only. The codons that encode asparagine 129 (N129) and aspartic acid 130 (D130) are shown in uppercase text.

TABLE 4
Annotated version of reference sequence SEQ ID NO: 5 (partial
ANP32B gene sequence)
SEQ ID NO: 5

| | |
|---|---|
| 1 | accgcggctg ccgagccgag cggcgccgcg cggggccgag tcgagccgag cagccacccg |
| 61 | cgccgcgtcg ccggtttaag cgcagtgcgg agccgcggcc gggggcggcg gtagtgagac |
| 121 | cagcgctgcg ctccagcccc cttttccctc catggtttct ctccgctccc gtgagtaact |
| 181 | tggctccggg ggctccgctc tcctgcccgc acgccgccag ccgccccgga ccgcgccgcc |
| 241 | ggcctctgcc tcgagcagac cccttccgac ggccctcgct gcgcaggctg ggagcctctc |
| 301 | ccccctccgc ccccgttgcg gaaagttaag tttgaagagg ggggaagagg gaaacatgga |
| 361 | catgaagaag agaatccacc tggagctgag gaaccggacc ccggcagctg taagcagaat |
| 421 | ccccttcggg ctccctctcc ccccgatgac ttgcatgtaa tggtggccac ctgcggggtc |
| 481 | ccagacgcgg gctcgccgac cccggcgcgg gcaagagcgc agctcgcggg ctcggacggg |
| 541 | aaaggcgggc gggcaggcgg gcgggcgcgg aggggggagc gagcgcgcgg gattaactct |
| 601 | ttggcgcccg cgggcccctg cggccgtcca gggaaggcgg tggggtgggg gaaagcaaac |
| 661 | tttgagctct tcgcccgccg gggtcctttc ccgaaggagc gcgtgtgcgt gtggccggcg |
| 721 | gcaagagagg gaggaggggg ttgtgtaacg ctgggagcca tgtttgcagc ctcccgagat |
| 781 | gcgcggccgg gcggaggccg ggcctacgga ggagccgcgc tgccagtccg gactgggccc |
| 841 | gctgcgggcc aggccggcct ccgcgtgccc ccggccggcc cgggaggggg ctcgggcgga |
| 901 | gcaggaggct cctccagcca ggtctcgcgg ttggcgccgg tccggccagg acgggagcgg |
| 961 | aaggacgaac agagggttct ttgcctccaa gggggacagg aagccccctc gggcgccggg |
| 1021 | gggcttgggc gggcgcggcc tggcgcgcgc gagctgggga ggggcgagcc cgggtggcgg |
| 1081 | aggtggcgca accgacgccc ctcccgaggc caagtttgaa aaaaggagtc ggttcccgcc |
| 1141 | atttttcaag cctaaaaata aaactttgtc tacctatctc tcgcgcccct ttcgaccccc |
| 1201 | aggccttccc cagtcccggt ctccaaatgc aggccgcgct cggcccgagc tagcagttgt |
| 1261 | ggcgtaagcg gcttctccct cccccccgcg ccgcggcggc ggagcccggg ggcccctgcc |
| 1321 | cgggaggttg gcccctcggt gtgggcgggc ggcggctcct cgcgtcagcc caggcgcgcc |
| 1381 | acaggccgac gcagcgccat gttggcgggc acccgtctcc ttccgccgcg tgagtcggcc |
| 1441 | tgcgggctct gtccgtccgg ccctgcccgc ccgcaccggc ggtggctctg agccgccgct |
| 1501 | cccttggccg agcctgcggt ttgtttggcg cgggcctcgc cagggctcgg ctcgcactgg |
| 1561 | cgggagccga gcgctccggc cgtcgccgac tcatctttcg ggcgcgctgc cgccccctcc |
| 1621 | ccccttctta aaagggcaac ggcaggcggc cgcctccgcc cctagtcgcg ggtacagacg |
| 1681 | ggcctcggct gtgcttgctt gccgggcggg cgctgccccg cacgtgcttc agagccgcag |
| 1741 | atccaggccg gtgctcgcct gccgcggctc cgtgagtcgc tggtgggcct tgaagagtca |
| 1801 | cgtctttatt ctctaccccg ccctccccgc gaagtagagc ggcttcccgc ggcagggcgc |
| 1861 | ggggccatgt ttcctggcgc cagcgctggg aggccgcgcc tccgccagaa cccctccgcg |
| 1921 | ggtgagtggc gtcccctggc agaggggcgg cccggggaaa gccctcacca ccccctcccg |
| 1981 | cccctgcgcc ccacactgga gacgggcgcg tttcgggcag tcaggtagcg ttgggtaaca |
| 2041 | agttgccaac ttgtgggaat tcgtcaagca ggctttccct ggtgtgccag gggttttaaa |
| 2101 | tctctgaggt ccccacaaat gaggtgcggc cctgctgcat ctggtgttcc cacctctccc |
| 2161 | tggaagacct ctggtggtgg gcgtctcgac tctgtcccct gagttttaag gttgagattt |
| 2221 | gaccgcggtc gtcccttccg tccatcagtc agacattttg tgtcggccac gtgctagtac |
| 2281 | attgaacctt taagcctctt cacactgaaa cttgcaacct aattttgaga agagttaaga |
| 2341 | catttgaatt attactgctc aggtccaaat caggtgaacg tgagtaatta aatttacccg |
| 2401 | agtttccaaa aagaaaaata cgtatatggt tctttaaagt gccttaagat ctgggatttg |
| 2461 | gattttagca atgtgtgtag aacagtatta aaatactcgg ctaagggctc tgtggctccc |
| 2521 | tctgagaaga aatacaactg agctggtggt gtatatggaa ttttttcgca catgaaataa |
| 2581 | ctaatactga tttttaaaat gcatatttat gtgcgttggg ccttttattt tttttcccat |
| 2641 | gaaatcaact taagccttac ccttttgcca aaacagtttt tagtcatgtg gctttttaaa |
| 2701 | ttaaaaggtc attttgccag ttgcactatt cacatttcaa gtactcagaa gtcccaaatg |
| 2761 | attaggggct gctgtattgg acagtactgc tctacagtaa ttcactggtt ttattaaata |
| 2821 | aatatttgcc aacagcatgc cagggcctgg aaaaacagtg aggcagagcg cagccccttc |
| 2881 | cattctgaag cctcagtaac cggtggtgcc tgttgcctag gtgcaagtta ggggccttgg |
| 2941 | gagagtgtca taggagccag ccaagattgt gggggcaaag ggtcccctag ggactgtaac |
| 3001 | agggagactg aaaacagtaa gagaggttct taggagagga aagccttgac cttcaactga |
| 3061 | ttgggggggg gtgctgtagg tttaaaggaa attttttttt ttttgtcttt ttgccatttc |
| 3121 | ttgagcagct cctgtggcat atggaggttc ccaggctagg ggtcgaatcg gagctgtagc |
| 3181 | caccggccta cgccagagcc acagcaacgt gggatccgag ccgcgtctgc aacctacacc |
| 3241 | acagctcatg gcaacgccgg atcgttaacc cactgagcaa gtgcagggac cgaacccgaa |
| 3301 | acctcatggt tcctagtcag gttcgttaac cactgcgcca cgacgggaac tccggtttaa |
| 3361 | aggaaatttt aaaagaagag tttaaaattt ttagcacaaa agcatcatct ttaaggtaag |
| 3421 | tgagaggttc caagatgatt ctaagtcagc aagtcttatg ggtttgttgt ggcgcgagac |
| 3481 | tggggaaagg ggaagaaccc tctctccatt gaatgtattg gtataacaca acgaaatgca |
| 3541 | caacagtgta ttccagatgt catcaagtgg aatagaagac tacttgagag taagtggagg |
| 3601 | cttgatcata aatgttttaa gaccagatag gcttttgagt atggcttttt ttttaatttt |
| 3661 | tttatttggg ggtttgaaga agaccctggt aacagtttac cttgtaagtc caaaacacct |
| 3721 | ttatatcaga agtgtctttt catagtggaa aaagtggctt aagatggcat ttttttttaa |
| 3781 | tttcattttt tatttttttt aatttttcat cataattttt tttttttatt ttcccactgt |
| 3841 | acagcaaggg ggtcaggtta tccttacatg tatagattac aattacagtt tttcccccta |
| 3901 | agatggcatt ttaacatatc tttgaaaaga ataatgatca gtgaaaatat aggcaaggag |
| 3961 | aggaaaattt ttatacaaaa tattagtatt aatttcactg agagcaaatt gtatagtgcc |
| 4021 | agaaaagtac cactgatatt gattcacata ttggtggtgg tatcagatgt gaagagacat |
| 4081 | gcaatatcaa gacgtaaacc aaatttagat atattccata attggaaaaa acacctgttc |
| 4141 | attatatgag gccctcatct ttctattttt ccaaaacatg gcactgttat taactatgga |
| 4201 | tctgggaaca tctgtccttc agtcttatgt tcaaaggata gaaagccatg cagttcctgt |
| 4261 | ggcttgtaat ctatgcccct gtgaaacact gtatgcttga ctgagacatt tcatatccca |
| 4321 | caaataatag tttttctcct aggcccaatt tactggagaa ttaagggttt tcttacatta |
| 4381 | caggttttca ttatgtattt ttaaatcccc actcccccac ttttggcatt tattgttgtg |
| 4441 | tctgttgtga ttttgagctc aatgggatat tctaattggt tgcaatagcc gctaattggg |
| 4501 | gttatttcct tatgtaagta gtgcctcttc attgaggata aattagatgc agataagcaa |
| 4561 | aacaatagta gatataaata agaaaaattt aaagaacctg aaaatctcac acacacccat |
| 4621 | cctaccatgt agatattctg gtgtagagtc cacgagaatg tttaattttt tccgaactca |
| 4681 | aattatactg ttaatgcata tactatctcg ttgctaatat agtagtaaac atctttatct |
| 4741 | tctatggcac aatttattat tttacagtct cattgttaat gtctatacag atcatgtaac |
| 4801 | cattcacatt aagtgaccag aaggattttt gcttttttaa atagtactga aatgaacatt |
| 4861 | tctacctatc agtaggatat gggtgtttcc ttccttcatg acacccaaac acatttgaga |
| 4921 | actactaatt gtaaattctg ttgtcatttt gaaaacgtac ttttaaaata atagtagggt |
| 4981 | ggaagagttt aagattggtt aactcatgag gctggaaatt gtcaaaggtg gcaggagtta |
| 5041 | ctttccacat ttttgttttt ttttgttttg ttttttgttt tgtctttttg tctttctagg |
| 5101 | gccacacccg aggcatatgt aggttcccag gctaagggtc caatctggag ctgtagccgc |
| 5161 | tggcctgcgc cataggcata gccatgccag atctgagtca catctgtgac ctacaccaca |
| 5221 | gctcacagta acgctggatc cttaagccac tgagcaaggc cagggattga acccacaacc |
| 5281 | tcatggttcc tattcggatt catttctgct gcaccatgac aggagttcca ctttccacat |
| 5341 | tattggatta tttcacttac tgcaataatc atgtattttc taactaaaag aattgataat |
| 5401 | gacctttacg cctccatatc tttttttccc tcttataaca aattataact gataaatggg |
| 5461 | gaaggtcact caagcaggta gggaaaattg attttttttt cccctagact aactagggga |
| 5521 | ataggaaatg agagcatatt ggagaggatg aaaagaaaat ttcttgatct cctgcggcta |
| 5581 | ctagctccaa tccccgtaac ttggctacag taaatttcaa agttttcact gactagttgt |
| 5641 | gagtgcatca gctttgctga tggctataga aaacaaaaaa attggatagg aaatgttttg |
| 5701 | gaattttgtt tttagctctt aaatgtttac ttaaattcat atttgacaaa aagctagttt |
| 5761 | agttgggaaa acgggggatc aagaagagta aatttggccc ccagtattag tctatccagt |
| 5821 | tactactttg gatcaaatag ctccctaagt tgaatgcaca aatagagaaa tacctgccca |
| 5881 | accttccagt attacatggt agccttgaga ggatttggga catgaggaag aaagagtagg |
| 5941 | gtgtttttcc ttgattagaa ccttctgttt atttggggtc taattaggag gccatgacac |
| 6001 | tatctctgcc ccgcatgctc caagcacctc ccttgttaag gcagtctaga ttccttcaca |
| 6061 | ctccaggaag caaattgggg agagggaggg tttaattatc tcatgctgct gtcatggtgc |
| 6121 | ctaaatacag acaccttttc accagggtcc tatttagatt ttggtctttt tagggctcgg |
| 6181 | atcccaagtt gctgtggcat aggctggagg ctgcagctcc gattcgaccc ctggcctggg |
| 6241 | aacctccatg tggtgtagga gtggtcctag aaaaggcaaa aaaaaagtaa ataaataaat |
| 6301 | aaaattaatc ttttgcagtt cctattgtgg cacggtgcag ttgaatctga ctagaaactg |
| 6361 | tgaagttgcg ggttcgatgc ctagcctagc tcggtgggat aaggattgga cgttgccccg |
| 6421 | agctgtggtg taggtcggca gctgtggctc tgattaaccc gtcctgggaa cctccatatg |
| 6481 | ccgcaggtgt ggccctaaaa aagaaaaaaa aaataaataa aatgagtctc acataaaaag |
| 6541 | acagcatgac attatttatg agaaacgtgt tcaacagaga agattataac ctctggtaca |
| 6601 | tccatatgct tagaatttct ttagtcgtta agaaatattt agagttgtta aaagcatatt |
| 6661 | aagtaatatt gcttcatctt gctaagacta aaaaaaatac caaaatagta agtgacaaac |
| 6721 | agtaaatgct ttgcattaat tgattatatt taggatcaga cggtagattc tcttcatatc |
| 6781 | caagtttccc ctggttcatc atctgctcag tcccctgcag tcctgattga tgtaaacctc |
| 6841 | tatgccattt ttttcttcat ttgacagttg tgaaggcttt tttttttctc agcttgcacc |
| 6901 | catggaagga acaaacccaa gtcacagcag tgaccatgct gagtccttcc ttaaccccta |
| 6961 | ggccaccagg caactcctga aggctttttc tcatattgag atatggtgat aactaaaaca |
| 7021 | gatatgacct taccatgaag cagtgactcc tccaccccta cacctgtgac acatggaagt |
| 7081 | ccccagtcca gggatccaat ctgagctata cctgggaaac accaggtttt taacccactg |
| 7141 | cacctggctg aggatcaaac ccgcactgcc ctgaggcaat actggatact aaaccagtgg |
| 7201 | caccacagca ggaactcctt agcagtgacc tttttaaaaa tagatcaata ttcttattat |
| 7261 | tgaaatgttt aaataaggtt atggaggagt tcccttatgg tgcaggaggt taaggatcca |
| 7321 | ttgccactgc agtggcttgg gtctctgctg tggtgtggcc acacacacaa aagttgtgga |
| 7381 | agtctcccag ccagtcaact ttccctgagg agaattcttg agaagactat aagaaagcaa |
| 7441 | gtgtgtgaaa tgttacataa gggataacgc attatatatt ctctaccatt tttgtcccac |
| 7501 | agcatatcct ggaaattagt ccctccttga tggacaatta aattgccttc taattttctc |
| 7561 | agttcttttt tttccagtta cttcttatgt cttatttata agcctttcgg agtgttgaaa |
| 7621 | ctaatgggtc acctttgggt actacaaacc caattctaat tggtactaag accaattctt |
| 7681 | cttaagtctc ctttgcagga tcccccaggg ttccatcctt ggttctcttc ctaggaattc |
| 7741 | catccttcta ctttttattg cttctgccct gacttccaaa tacagaactt aaaccgagga |
| 7801 | ctcccctgaa ctccagacct gcatgtctag ctgactgggg atactttcat ctggatatcc |
| 7861 | catggaaaca aacttggtat cttttgtagt agtcattaca tatctgcctc tctcccaaaa |
| 7921 | agaagccact ccttcctatt tccatctagc tacatagtcc ccatttctca aacgaacctc |
| 7981 | agttctttat catcttttgt accccacagt taaacttgca gattttgacc actttaactt |
| 8041 | ttttcccctc ccagtttctg ttatttggtt cagcaaactt aattctctca tatttgaatg |
| 8101 | ttttatggta gtcttttttt gggctatgcc acggcatgtg gaagttcccc gtccatagat |
| 8161 | tgaacccaca cctgagccac tacagtgacg ttgctgaatc cttaacccac ttcgccgcaa |
| 8221 | gggaactcca gctttatggt agtctttgat gaccttagtt aaagcagaat tttgtcagtt |
| 8281 | gagaacaatg tccaagagtt gatttcacag tggactcgag gtataatgtt taaactcctc |
| 8341 | tgtggctttt gacatattgg tgtcatcttg tataatattt ttgaattctc tgtcagataa |
| 8401 | tagttgtaga aaatctacca aatgaaatgg aaatgtttag ctgtattgca atactactta |
| 8461 | ggtgagtggt gattacagtg gccttttttt tttttttaat gattagttgg ttttagggtt |
| 8521 | ttttttgttt tttgttttct tttgagggcc acacccccag tgaaatatgt tagttcacag |
| 8581 | gctaggggtc taatcagagc tgcagctgct ggcccatgcc acagcaccta gggatctgag |
| 8641 | ccacatcttt gacctacacc acagcttaca gcaatgccag atcctttaac ccattgagtg |
| 8701 | ggtccaggga tcaaaacctg agtcttcctg gatactggtc aggtttgtga cccactgagc |
| 8761 | cacaatggga actcctgttg gaaagcttgc ctggaggtta cttcctcttt tatttttttg |
| 8821 | gccaccctgc agcacatgga gttcccaggc cagagatcaa atcctagcca cagttgcaac |
| 8881 | tttaaccctc tgtgcctggc cagggatcaa acctccttcc caagaggctg ttgagtcctg |
| 8941 | ttgagccaca gcaggaattc cagtttgttt gtttgtttgt ttttctttct ttttttttac |
| 9001 | cttagggctg caccgtggca tgtggaggtt cccaggtact aggggtcaac ttggacctgc |
| 9061 | agctgccatt ctgtgccaca accacagcaa ctccggatct gagcctcatc tgtgatctac |
| 9121 | accacaggtc acagcgtgtt tgtttgtttt ttaatacaca agaagtacaa agagttccca |
| 9181 | ttgtggctca gctacctaaa aacgcaattg gtatccacga ggatgcaggt ttgatccttg |
| 9241 | gcctacctca gtggcttaag gatctgtcat tgtcacaagc tgtggcatag gtcacagatg |
| 9301 | atgcagcttg gatctggcgt ggctgtggtg tgggctgaca gctgtagctc ccactccacc |
| 9361 | cctatcctgt gaacttcata tgccacaagt gcagcccgaa aaaggaaaaa aaaaaaaaaa |
| 9421 | aaaaaagagg aatacatgct tagtacagat aggaataaat tgcaacgatc cttgtcattc |
| 9481 | tccttcaccc ttgcccagag gttactttct cagcatttac tctgtcccaa gcctactttc |
| 9541 | tacagaagtt ccacttcaca gatgaggaaa ctgaggcaca gagaagttaa ataacttgcc |
| 9601 | caaggtcaca caggttgtaa gtgacaaagc agatagaatg gaacctgtat gcgtcctgaa |
| 9661 | agcaggacgc agacagggga gtgagaagtg ctgaagaagc aagtgagaag ttgtggtgtt |
| 9721 | atcggtgaat ctctaaatct gtttgcttct ctaactgggg ggagggtatg gtggtacaaa |
| 9781 | gaataatgag taaattctcc atagatgata gcaattacta gagcagtatc cttggacttt |
| 9841 | gtatgagagg atgaagatgt tactacctcc tgttttcatt ttaaaactgt cagcaagcat |
| 9901 | ctccttccct tatacatgct tgattaatgc tattaaatgt gttagattca ctgcaagaat |
| 9961 | gcctttggca gttaaaacta atttgcatca gtgtaaattt atcaaaacgt gtttaaaact |
| 10021 | tgcaagcttc agagtcagcc acagagcaag aacagcagta gagactatca tggtagatag |
| 10081 | tcacacagta gactgtttat tagataatca gtattcaaaa ttctcagata tacttttgga |
| 10141 | tggatttgaa ttctgaatat taagatctcc aaagaaggag attttatggc ctaaaaagaa |
| 10201 | cctaatatct aacagtttgc ccccccagaa atagctttct cacatgtaac acagaatgat |
| 10261 | ctttgaatta agacctcctg tgtcttgatc ttttcctaat atcaagataa tctaagtaaa |
| 10321 | aattaataaa accacaagtg gaaatgtgta gctgagaaac ctgtagccaa aatctcttag |
| 10381 | tgatcttgat gggagaagta aagctttcct cttttctttg tgtcagtctc ctgcgccttg |
| 10441 | agctaggctt ggcctaaaag ggttgctggg gagagtggtt tgggaaggga taatcttcac |
| 10501 | tctgcatttg tagttaaagc tactccttgg ggaagagact taggaaaaag aataaggggg |
| 10561 | gtgtcatttc ttcacttaca tcctaaggac tatagtttct gtctctggaa agtaaagaat |
| 10621 | ttaaatcttt gttgtacatg gtgtgacatc ccacccttga tatctatcag aatattgtta |
| 10681 | ctaaaggctt aatcgttttt cctttattta tagctgagcc aggtctaagt agagcctgcc |
| 10741 | cctatatggt tggagttttt gctttgttct ttgttttcta tatggttttg aaagcttgtc |
| 10801 | tcctcctctt tgtcacccac aggttcgaga acttgtgctg gataattgca aatcaagtga |
| 10861 | tgggaaaatt gagggcttaa cagctgaatt tgtgaactta gagttcctca gtttaataaa |
| 10921 | tgtagggttg atttcagttt caaatctccc caaactacct aaattgaaaa aggtaagtgc |
| 10981 | tttttcttta acagtaaaag agagaaccat tctggggaag gggaaaatat atgattctat |
| 11041 | ctgtaaggaa gcatttagtg tagcagaaag cgcatggcct ttggaactgg gcagatcagg |
| 11101 | gattgaattc cagctctatc atttccctga tgggtgatct tctgcatgtt acttaatctc |
| 11161 | tccaaggctc aatttcctca cctgtaaact ggtgatacca cctgaccttc cagaatggct |
| 11221 | gtggtgtttg aaagtagtgc atgttgaatg ctcaataaat aaatagtggt tatgaagaat |
| 11281 | aattgttagg gatttattgt aatgtcagct acttttgttc ttaatatgct ctggcttagg |
| 11341 | aatcacaggt gcctcccact cttcatgttt ttttgttaag gccaagttta agtaaaggct |
| 11401 | tttcaaattt gtactccatg gtatttcaaa ctcagtatat aattgttatt tttgcttttt |
| 11461 | agggccacac ccacggcaca tggaggttcc caggctaggg gtcaaatcgg agctgtagcc |
| 11521 | acgagcctat accatagcca ggacaatgcc agatccttaa cccacagagc aaggccaggg |
| 11581 | atcgaacctg catcctcatg gatgctagtc agattcgttt ctgctgagcc acagtgggaa |
| 11641 | ctccccatta tgtaattctt aatgactggt aagtttaaaa tgtgatttac tccaagagtt |
| 11701 | cattttaaca acattgctgt atttccctgt cctacttgat agctcattgt tagtgcttta |
| 11761 | ttggtctaat tataatcaat cattgtcacc ttcaacctcc ccctcaccca aaaaacatgt |
| 11821 | ttttttcatg tccttgcaca tttacctgtc acagagcccc cagtatgtca gaagtggaag |
| 11881 | aaacttatat cttgtggcct cggcctttgt tttataggca cagtagtaga tccagaaagg |
| 11941 | tatggtaatt tgcctaaagc cactcacgaa agtggttagt agtagagcca gaattagagc |
| 12001 | tcatatcttc tatttgctaa cccagtacct tttccattgg cagtgttgca gaattctcca |
| 12061 | ctgtttacct catctctgta tcagggagtt tgagctgaac ctttaatcta aaaaagatac |
| 12121 | gcatagaaat ctgaagtcaa atttaggcca aagacaagta agctgttcct atagcctgcc |
| 12181 | tgcttgaatc ccgagaggga cttttttttg gttaagtata tgaattaaaa atcaggagtg |
| 12241 | ggagttccca tcatgacgaa gcggaaacaa atccgactag gaaccatgag gttgcgggtt |
| 12301 | ccatccctgg cctctgtcag tggattaagg atctggcgtt gccgtgagct gtggtgtatg |
| 12361 | ttgcagatga acctccatgt gccgtgggta tggccctaaa aagcaaaaaa aaaaaaagaa |
| 12421 | aaaagaaaca ggttctggga gaaattttgg tgtgtaaatg tcacaagttt cctaaattta |
| 12481 | aacattgatt tcatttcaaa ataaatatgc cactaaatgc tttggcaaac tgccttgctg |
| 12541 | tccctactcc ttaagatatt tgtgttgaaa ggatatgaat attatacgta tcgatattct |
| 12601 | aacaaccaag agaaatggtc tctattttga atttctaaaa cctgtgtaaa gctaataaca |
| 12661 | cttgtgtctt tgttggtata gagatgtatt ttaaaatcta gaccacagtt agatctttta |
| 12721 | ttggactatc ccattggaag gaaaaaaaag ttttgaacta ctgcattttt tctcttctga |
| 12781 | aatacttaaa aacaaccaag cagcagactt actttttttg ttgttgttgg ttttttgttt |
| 12841 | gcttttttta gggctacacc tgcaccatat agaagttcgc aggctaggga ctgaatcaga |
| 12901 | gctgcagctg ccgacctacg tacgccacag caacacctga tctgagtcac atctgtgacc |
| 12961 | tacgctgtag ctcttggcaa tcaatgccag atccttaacc cagtgagtga tgccagagat |
| 13021 | tgaacccaca tcctcatgga tactagttgg gttcttaacc tgctgagcca caacaggaac |
| 13081 | tcccagattt actgtttttt aaactcattc tttcaacaaa tattttaatt tttagaagtt |
| 13141 | cctctgcatc atgtagttca tttcattgca tgcccaacta aatcatgtaa gttttcatgc |
| 13201 | catttatcac tgtaacagct atgtaatctt gggcacttac ataatctcaa tagttcattt |
| 13261 | tccttttctg taaatagggg tgataattct acttgctccg taggatttac tgtgagatca |
| 13321 | gatgagttca tgtatttcta gtgctttgaa tagtgcctta agcacttggg tacttagtac |
| 13381 | ttaagtactg tgtaaaggtt agatacggtt actgttgttg ttggcactta aaattgaaac |
| 13441 | tagctatatg gccttagttt tcgtatctgc aaaatgaaga gtttgaatta gatgctctta |
| 13501 | accatcttat ttttccacgc ttcagtttct tttactctta agattcacat attccttggt |
| 13561 | gaaggttcgt tccttcattg acagaccagt tgccgtgctg cttgatcggc agctaagaaa |
| 13621 | ttagagtagg ttcttaaaaa ggcttcttgc caagtttgtg caaaaagtat cctctgagga |
| 13681 | gttggttttg tcagctttga acctattgcc ccactccctt ttctgaggta atggagcttc |
| 13741 | tttaaaggct tgtaccattc ccatagcttt cagggcagca tttctggtag cttccagatg |
| 13801 | tagaacccca tggcaagtca gcactgtaca gaaccagatt aggtttcaag tgggcattgt |
| 13861 | gagctttgct tggcacacag aacaagctgc ttagaatgca cagaccattt acccttcttg |
| 13921 | gcaggtatcg aatattacaa cttctcaaag aattctaatt tgctttgacc cactttttag |
| 13981 | gttagaaaat gtattcactg atattaggga ccagcaaagt gttctaatta gcagctggtt |
| 14041 | aagaaaacac aactcttttc attcaaattg tctctccttc cccaaagtgt tctggggatc |
| 14101 | tgtaaactta tttgggttta atgaaacaaa atgagcatat gtgccctaat aaaaaataga |
| 14161 | gatcatttga tcttggttgg tgtttaagaa ctatattggt tacttctaac accttaatga |
| 14221 | tatgtgtttc tttttgtgtt tgtgtgccgt gtgcattttt ccctttaaca gcttgagctc |
| 14281 | agtgacaata gaatctatgg aggtctggat atgttagcag aaaaacttcc aaatctcaca |
| 14341 | catctaaact taagtggaaa taaactgaaa gatatcagca ccctggaacc tttggtaagt |
| 14401 | agttgagaat ttggaaaaca ggactttctg gtcattttca ttttcatatt tctttatggt |
| 14461 | gagggaaata attgaaagtt ataatggaga aagtggtgat ttttttatag taaacttttt |
| 14521 | atagaaagag tccagtttta aagatcagag cctcactagt tatgtgttcc tgtggcatta |
| 14581 | ctctcccttt ctgattatta tggtggcttc cttcaaaatg cttttctttt gtccttttta |
| 14641 | aatgtcatat tgactatagc cttaaacatc catcttcaaa ttttttcccc agactggcca |
| 14701 | ggctgttctg tacttttgta cttttccaaa aaggctgttt atgttgggtg gcatatctga |
| 14761 | ctgtagaagg taccatccaa gggcagctcc agtgagtttt aggcctttta attcatgatc |
| 14821 | ttgtgctcta gctggatcat tgttgcctaa agttttgcat gggaacccct aaattctcct |
| 14881 | cttagtcctg gaataagctg tggaaggtta agtttaagtg ctacttgggt tacagacttg |
| 14941 | ctgtgtgacc ttgggcaaat tgcttcttct ctctgggcct cagtttcctc acttatgaag |
| 15001 | cagaaaggat aatcctgcct tacttcttag gaattctgag agtaaaatag gtatttattt |
| 15061 | accttgcttt gtaagttggg aagctaagct aattgggggt ttagtccatg tggaagttac |
| 15121 | aagtggccag aaaagagctt cagaaggcaa gggttcatgc agagtagggg gctttggctg |
| 15181 | ccacctgaaa attccgtgat ttcaagattg ttaaatgact tttcccccta aatatgacat |
| 15241 | tcttgtctat tttaataagg ctaatgaaac tcagatttgt tttataaccg cccaaagaag |
| 15301 | ttttgtggct tctttctggg tttttgtttg tttttctgtt gtacataaag tcagtatttc |
| 15361 | catctgattt aacttgcaga cattaattgg aagagaggct acacagtgga taagagctgt |
| 15421 | ctttcaggat ccagcagagt agaacccaag agtatgagac atccttctgc tatctctatc |
| 15481 | ttttctggtg tcacaatata tttaaatatt tggatttcat tcacttacac agaatgcttt |
| 15541 | attgatattt cgagggaaga taatgtaggg tcagataatt gagactcctc caatatagat |
| 15601 | ttaatcccca tttactaagt ccctttacat ttcattaagt ggatcacatt gagctcattt |
| 15661 | ctcattaagt gttgggagca gcctgttgat tacctttgct ccatgaattt tgcccctttt |
| 15721 | ttcctgttaa actcagatac ttgagggcac cctatttttt taaaattagt tttcctccaa |
| 15781 | atcaatcccc atttccaatg ttaaagcaaa catttattgt gttttgcttt attcagggac |
| 15841 | attaacatct gcccattgta tcctcatatg atgcagagaa gagaaactgt cctgttagaa |
| 15901 | aggaggaaac caagtttaag ccgcaggata aatggcaagt agggattcaa atccaagtcc |
| 15961 | atcattcttt gcactgccct tgggacctgt cttttctttt aatttacacg ttttttttcc |
| 16021 | tacccaaatt gacctaagct gggagttaga acaggagcac taaattaagt gcttaggctc |
| 16081 | tcccagtcag atgggctgca taaaagcctt tgtttgtgtt aatgacgagt ctatcaattt |
| 16141 | agggtcatag aaaacttagg aatccctttt ctctagaaca gagtaccagg cagtaccttt |
| 16201 | ccttgctagg tcactggcca ttgcctaatc tcatggcaac cctagtcatc tttgattttt |
| 16261 | ttcttggagg cttttgtcaa tgtcctaaca ttcagaggca gagcagaagc aaatgttgtg |
| 16321 | tgggagttgt ttgcatgggt gtttctgacc tgggcggtga tgtggttagg gccctttggt |
| 16381 | ggatatgaaa gtctgaatcc ccttggacag tgaaatatga gaaataatca tttaaatatt |
| 16441 | taccatttgc agaggatctg ctttgtacga tacaccaaag tggatgcttt atgtgtgttg |
| 16501 | ttcataatcc tcaacatttc tccagagtgg agagacatag catggcccac gttttacaca |
| 16561 | tgaggaagct gaggcttagg agaggttgaa tcccttgtcc agagtcacac acctggcagg |
| 16621 | ccagtgacag agccacagtg ccacacaccc agggctatct gggaaagcct gagtgcctgc |
| 16681 | cattcttctg aacttgctgt tcagattcct ggatgtacta ggtgggtctc ctgaagttac |
| 16741 | aacttaagga taaataaaac tgtaggagaa agtgtggggg cgggggaaat gttgggtcca |
| 16801 | ttttgacttc tgaaagtatt cttcagttgt gaaaaacacc atcctcatcc tcatcaacca |
| 16861 | catacatttt gaacaccgtc tactttgagc ccagttctta cagtcaatgc aatgattgtg |
| 16921 | aatgcctctg cttactacag ggacattaat cgagagacaa agaaggcatc aattcaaact |
| 16981 | tccttggaca ccttttttaa gagacctggg ccatccaaac catcccccaa agcccttcta |
| 17041 | aattctaaga aggcaagcaa aattattgtt aataaaatgt ttaatgttta ttatggatag |
| 17101 | cctattagtt ttaggccaaa tttctgagat ggtagagttg tgaatttttt ttctttcttt |
| 17161 | ttttttaaag ccgtatttac acattacatt gggatcctga atatctgtgt gtagtgtatt |
| 17221 | ttggaccttt aaagcacagt tttactgctc taaacaattt tagagttcta ccattattag |
| 17281 | gagttttgtt gatgtttatt tggtttcact ttgttttcta gcatattgtt tggcatgtct |
| 17341 | gaattattca tatgggtcct ttctaaagaa gtggccaaaa atgtcacgga ttcagagttt |
| 17401 | cagaaacagg aggccctgtg ttaggctagt caatagagtt agaaaatgtg cttggggcac |
| 17461 | tgttgagcat taagatcagg ggttgtatgc atttttttgt acatccattc ctagggaata |
| 17521 | atattgaaca gtaaatggct gcttccttta agtgaagaat ttatactgag cctctgtggg |
| 17581 | gaaagctgcc taaatttact tgtggttcta tattagctgt tacagagtca acccttttgt |
| 17641 | gaaaagagtt ccccagttgt ctggggtgct atttcaacat tttttccccc tctttggcaa |
| 17701 | gaacgcattc ctgggcttcc ggagttcagg gactgtctag taggaaccta atctgaaagc |
| 17761 | tgcatgtcat aaaagcgttt ctccctgtga gccctagggc tggggacaac tatcttaaag |
| 17821 | aaagacctca ggggactgat ttacataagc acattattgg atcagcgacc tcttggatca |
| 17881 | gtgtacgtgg ctgttttgag gagtacattt gcctgttgct cttaactctg ttctgggtct |
| 17941 | gtgctcccaa gggctgttca gtgagaataa aatcaatgtg ggcacctctt tgatgaagaa |
| 18001 | aatgaaggtt tgtgagtgca ggagtcagca accctttgct ctcaaggacc agatagtaga |
| 18061 | ccacctaggt tttgtaggcc ttagtctttg ttacagccac tccactttgc ccttatagct |
| 18121 | caagagaagc cacacaaagt tcctaaacaa gtgagtgtgg ctgtgttcca gtgaaagtta |
| 18181 | gtttacaaac acgagaggcg ggcaggatgg gccagcacag tttgccaagc cctgctttaa |
| 18241 | agcagagatt ttgacatcat tttgtaactc ccaagaatta tacagacacc gtagatcctg |
| 18301 | aagacaaggt tttgtttcat ctatatctct tcccaggccc ttgtgaatat tgaaatagat |
| 18361 | ttccaatgaa gtatcaaaat ccttaaacat caacatttat tttctgcaac ttacttcatg |
| 18421 | aagagatcct cttaatattt ttttagagaa tggtaaatct tttgattcaa aacctttaaa |
| 18481 | tgctaaattt gtgggagttc ctgttgtggt tcagcaggtt tggaacccag cacagtgtct |
| 18541 | gtgaggatgt cagtttgatc cctgggctca ttcagtggat taaggattca gcagtgctgc |
| 18601 | aggctgtggt gtagtcacag atgcagctca aattcagtgt tgctgttgct gtggtgtaga |
| 18661 | ccacagctgc tgctcctatt cagctcctca cccgggaact tccatatgct acaggagcag |
| 18721 | ctttaaaaag aaaaaataaa taaaggttaa acgtaggaag ccatatatcc atatgccccc |
| 18781 | aggcccccct cttttgcttt ttagggccac acctataaca tatggaagtt cccaggctag |
| 18841 | gaatagattg tagctgcagc tgctggtctc tgccacagca gtttggggat ctgagctgtg |
| 18901 | tctgtgacct acacctcagg tcatggcaat gctggatcct taaccccact gagtgaggcc |
| 18961 | agggatcgaa ccagcatcgc atcctcatgg atactagcag ggttcctaac tcgctgggcc |
| 19021 | acaatgggaa ctccccacag gcccccgttt taaaaggcat cagactacgt agtatttcac |
| 19081 | attttgagtt ttctttttac aggtgtgccc ccttttttgc tctctgtgag ggtctcttgg |
| 19141 | cattttgtta ctatctcctt acttacctat agtactacct atgtcctccc aatcttgtaa |
| 19201 | tttaccctta gtcaaaactt aacagacctg gcattgaaat actgatacac tgatataata |
| 19261 | atgaagacct gagtcagatg ttttcctgtc cacttcacct gctcagaaaa cttggggggc |
| 19321 | caagtttcta gatcttaact ggtttagtta gatgaaactg aagttctaaa caaatgaggt |
| 19381 | gttgcaacaa aacttgaagg tatggtaaaa tcagaaaaat ttttaaagtt ctgataatag |
| 19441 | aacttttccc caaaaatcaa gaaagaagag tgtaatgttc agtcttttcc ccaaaaatca |
| 19501 | agaaagaaga gtgtaaagtt tctctacttg ctgagtttca actctgagtc ccctttcttt |
| 19561 | ttcccccagc ctacattgac tcctgccttt ctgaaaaata gctgcatccc cacactttgt |
| 19621 | gctttactag actcctttgg agtatttaca tttatgtctc caagtagaaa tctcaagaat |
| 19681 | ttcaagatat cgattgaatt aactcatttg atttaatttt ccccctccta tactaccacc |
| 19741 | ttagagcact gactgcttga taatcaagcg ggttcttact ggtcatttga ttactcatac |
| 19801 | attaaaacta gttatttata actaaataaa tttaaccaga tatattaaac tctgtttgat |
| 19861 | ttgtctcaca ctttaaaaag atctagtttt aatgtttcaa aggcatattt cttaagacta |
| 19921 | ctcatgctgc caaaagaaaa ttctctagag gatagtggta gaaggtaaaa agtagttaaa |
| 19981 | aatctagtga cctaatagaa aggttgcgaa ggacttgaaa tgttttccta tgtaaggctg |
| 20041 | agtaggtagc agctaattga atatagagcc tagagtttta cctctttagg aggtgggttt |
| 20101 | tgttgcttgt tcctttggtt taggaggtac attttttgat ccttttcttc agaaaaagtt |
| 20161 | ggagtgtctg aaaagcctgg atctctttaa ctgtgaggtt actaacctgA ATGACtatcg |
| 20221 | agagagtgtc ttcaagctcc tgccccagct gagctacctg gatggctatg acagagacga |
| 20281 | tggtgaagcc cctgactcag atgccgaggt ggatggtgtg gatgaagaag aggatgatga |
| 20341 | ag |

The amino acid sequences for porcine ANP32A and ANP32B proteins are provided below in Table 5. In human and chicken ANP32 proteins, the asparagine (N) residue at position 129 (N129) and the aspartic acid residue at position 130 (D130) have been identified as being required for interaction with the influenza A viral polymerase. Locations of the corresponding asparagine and aspartic acid residues in the porcine ANP32 amino acid sequences are indicated in the Table 5 below for SEQ ID NOs. 6-10.

TABLE 5
Porcine ANP32 protein sequences

| Protein | SEQ ID NO. | Amino acid sequence | Position of ND |
|---|---|---|---|
| ANP32A | 6 | MEMDKRIHLELRNRTPSDVKELVLDNCRS | N129, D130 |
| (ENSSSCT00000005475.4) | | NEGKIEGLTDEFEELEFLSTINVGLTSVA | |
| Sus scrofa | | NLPKLNKLKKLELSDNRISGGLEVLAEKC | |
| | | PNLTHLNLSGNKIKDLSTVEPLKKLENLK | |
| | | SLDLFNCEVTNLNDYRENVFKLLPQLTYL | |
| | | DGYDRDDKEASDSDAEGYVEGLDDDEEDE | |
| | | DEEEYDEDAQVVEDEEDEEEEEEGEEEDV | |
| | | SGEEEEDEEGYNDGEVDDEEDEEEPGEEE | |
| | | RGQKRKREPEDEGEDDD | |
| ANP32A | 7 | RCLLVCLLSPPRSWAPFLTPFTPALSFQV | N139, D140 |
| (ENSSSCT00000070641.1) | | KELVLDNCRSNEGKIEGLTDEFEELEFLS | |
| Sus scrofa | | TINVGLTSVANLPKLNKLKKLELSDNRIS | |
| | | GGLEVLAEKCPNLTHLNLSGNKIKDLSTV | |
| | | EPLKKLENLKSLDLFNCEVTNLNDYRENV | |
| | | FKLLPQLTYLDGYDRDDKEASDSDAEGYV | |
| | | EGLDDDEEDEDEEEYDEDAQVVEDEEDEE | |
| | | EEEEGEEEDVSGEEEEDEEGYNDGEVDDE | |
| | | EDEEEPGEEERGQKRKREPEDEGEDDD | |
| ANP32B | 8 | MDMKKRIHLELRNRTPAAVRELVLDNCKS | N129, D130 |
| (NANS) | | SDGKIEGLTAEFVNLEFLSLINVGLISVS | |
| (ENSSSCT00000005912.4) | | NLPKLPKLKKLELSDNRIYGGLDMLAEKL | |
| Sus scrofa | | PNLTHLNLSGNKLKDISTLEPLKKLECLK | |
| | | SLDLENCEVTNLNDYRESVFKLLPQLSYL | |
| | | DGYDRDDGEAPDSDAEVDGVDEEEDDEEG | |
| | | EDEDKEEEEDGEEEEFDDEEDEDEDEDVE | |
| | | GEEDEDEVSGEEEEFGHDGEVDEDEDDED | |
| | | EDEDEDEEEEENEKGEKRKRETDDEGEDD | |
| ANP32B | 9 | MDMKKRIHLELRNRTPAAVRELVLDNCKS | N129, D130 |
| (NANS) | | SDGKIEGLTAEFVNLEFLSLINVGLISVS | |
| (ENSSSCT00000054395.2) | | NLPKLPKLKKLELSDNRIYGGLDMLAEKL | |
| Sus scrofa | | PNLTHLNLSGNKLKDISTLEPLKKLECLK | |
| | | SLDLFNCEVTNLNDYRESVFKLLPQLSYL | |
| | | DGYDRDDGEAPDSDAEVDGVDEEEDDEEG | |
| | | EDEDKEEEEDGEEEEFDDEEDEDEDEDVE | |
| | | GEEDEDEVSGEEEEFGHDGEDEDEDEDEE | |
| | | EEENEKGEKRKRETDDEGEDD | |
| ANP32B | 10 | MDMKKRIHLELRNRTPAAVRELVLDNCKS | N129, D130 |
| (NANS) | | SDGKIEGLTAEFVNLEFLSLINVGLISVS | |
| (ENSSSCT00000068404.1) | | NLPKLPKLKKLELSDNRIYGGLDMLAEKL | |
| Sus scrofa | | PNLTHLNLSGNKLKDISTLEPLKKLECLK | |
| | | SLDLENCEVTNLNDYRESVFKLLPQLSYL | |
| | | DGYDRDDGEAPDSDAEVDGVDEEEDDEEG | |
| | | EDEDKEEEEDGEEEEFDDEEDEDEDEDVE | |
| | | GEEDEDEVSGEEEEFGHDGEVDEDEDDED | |
| | | EDEDEDEGGLLRALWGNVSLFFGFLGPHL | |
| | | RHMEIPRLGV | |
| ANP32B | 11 | MKMKSVGRKKNLDMMEKSMKMKTMRMKMR | |
| (NANS) | | MRMKECGADCAKFQKSELEYKENRKALER | |
| (ENSSSCT00000062686.2) | | PYTSKHSWGKTYGEHKRHLEFSHAQYREL | |
| Sus scrofa | | QKFAEEVGIFFTASGMDEMAVEFLHELNV | |
| | | PFFKVGSGDTNNFPYLEKTAKKGRPMVIS | |
| | | SGMQSMDTMKQVYQIVKPLNPNFCFLQCT | |
| | | SAYPLQPEDVNLRVISEYQKLFPDIPIGY | |
| | | SGHETGIAISVAAVALGAKVLERHITLDK | |
| | | TWKGSDHSASLEPGELAELVRSVRLVERA | |
| | | LGSPTKQLLPCEMACNEKLGKSVVAKVKI | |
| | | PEGTVLTLDMLTVKVGEPKGYPPEDIFSL | |
| | | VGKKVLVTIEEDDTIMEESVENHGKKIKS | |
| ANP32B | 12 | MPLELELCPGRWVGGQHPCFIIAEIGQNH | |
| (NANS) | | QGDLDVAKRMIRMAKECGADCAKFQKSEL | |
| (ENSSSCT00000045745.2) | | EYKFNRKALERPYTSKHSWGKTYGEHKRH | |
| Sus scrofa | | LEFSHAQYRELQKFAEEVGIFFTASGMDE | |
| | | MAVEFLHELNVPFFKVGSGDTNNFPYLEK | |
| | | TAKKGRPMVISSGMQSMDTMKQVYQIVKP | |
| | | LNPNFCFLQCTSAYPLQPEDVNLRVISEY | |
| | | QKLFPDIPIGYSGHETGIAISVAAVALGA | |
| | | KVLERHITLDKTWKGSDHSASLEPGELAE | |
| | | LVRSVRLVERALGSPTKQLLPCEMACNEK | |
| | | VGSDPARCFCWEGLQWACLRGGL | |
| ANP32B | 13 | MPLELELCPGRWVGGQHPCFIIAEIGQNH | |
| (NANS) | | QGDLDVAKRMIRMAKECGADCAKFQKSEL | |
| (ENSSSCT00000044227.2) | | EYKENRKALERPYTSKHSWGKTYGEHKRH | |
| Sus scrofa | | LEFSHAQYRELQKFAEEVGIFFTASGMDE | |
| | | MAVEFLHELNVPFFKVGSGDTNNFPYLEK | |
| | | TAKKGRPMVISSGMQSMDTMKQVYQIVKP | |
| | | LNPNFCFLQCTSAYPLQPEDVNLRVISEY | |
| | | QKLFPDIPIGYSGHETGIAISVAAVALGA | |
| | | KVLERHITLDKTWKGSDHSASLEPGELAE | |
| | | LVRSVRLVERALGSPTKQLLPCEMACNEK | |
| | | WDFLAAFILKMHVHWEIKMTVKRNKLPSR | |
| ANP32B | 14 | MVLKDSDLIAVALAFLFSLRECGADCAKE | |
| (NANS) | | QKSELEYKFNRKALERPYTSKHSWGKTYG | |
| (ENSSSCT00000055524.2) | | EHKRHLEFSHAQYRELQKFAEEVGIFFTA | |
| Sus scrofa | | SGMDEMAVEFLHELNVPFFKVGSGDTNNF | |
| | | PYLEKTAKKGRPMVISSGMQSMDTMKQVY | |
| | | QIVKPLNPNFCFLQCTSAYPLQPEDVNLR | |
| | | VISEYQKLFPDIPIGYSGHETGIAISVAA | |
| | | VALGAKVLERHITLDKTWKGSDHSASLEP | |
| | | GELAELVRSVRLVERALGSPTKQLLPCEM | |
| | | ACNEKLGKSVVAKVKIPEGTVLTLDMLTV | |
| | | KVGEPKGYPPEDIFSLVGKKVLVTIEEDD | |
| | | TIMEESVENHGKKIKS | |

Ungulate animals and offspring thereof comprising at least one modified chromosomal sequence in at least one gene encoding an ANP32 protein are provided.

Ungulate cells comprising at least one modified chromosomal sequence in at least one gene encoding an ANP32 protein are also provided.

The modified chromosomal sequences can be sequences that are altered such that an ANP32 protein function as it relates to influenza A infection is impaired, reduced, or eliminated. Thus, animals and cells described herein can be referred to as “knock-out” animals or cells.

The modified chromosomal sequence in the gene encoding the ANP32 protein reduces the susceptibility of the animal, offspring, or cell to infection by a pathogen, as compared to the susceptibility of an animal, offspring, or cell that does not comprise the modified chromosomal sequence in the gene encoding the ANP32 protein to infection by the pathogen.

The modification preferably substantially eliminates susceptibility of the animal, offspring, or cell to the pathogen. The modification more preferably completely eliminates susceptibility of the animal, offspring, or cell to the pathogen, such that animals do not show any clinical signs of disease following exposure to the pathogen.

For example, where the animal is a porcine animal and the pathogen is influenza A, porcine animals having the modification do not show any clinical signs of influenza A (e.g., fever, lethargy, anorexia, weight loss, nasal and ocular discharge, cough, sneezing, conjunctivitis, and/or breathing difficulties) following exposure to influenza A. In addition, in porcine animals having the modification, influenza A nucleic acid cannot be detected in the nasal secretions, feces, or serum; influenza antigen cannot be detected in the tissues of the animal (e.g., in lung tissue), and serum is negative for influenza-specific antibody.

Similarly, cells having the modification that are exposed to the pathogen do not become infected with the pathogen.

The pathogen can comprise a virus. For example, the virus can comprise an Orthomyxoviridae family virus.

The virus can comprise an influenza virus, and preferably comprises a type A influenza virus. The type A influenza virus can comprise an H1N1 subtype virus, an H1N2 subtype virus, or an H3N2 subtype influenza A virus.

For any of the ungulate animals or offspring described herein, the animal or offspring can be an embryo, a juvenile, or an adult.

Similarly, the cell can comprise an embryonic cell, a cell derived from a juvenile animal, or a cell derived from an adult animal.

For example, the cell can comprise an embryonic cell.

The cell can comprise a cell derived from a juvenile animal.

Any of the animals, offspring, or cells can be heterozygous for the modified chromosomal sequence in the gene encoding the ANP32 protein. Animals, offspring, and cells that are heterozygous for the modified chromosomal sequence in the gene encoding the ANP32 protein include animals, offspring, and cells that have a modified chromosomal sequence in one allele of a gene encoding the ANP32 protein and no modified chromosomal sequence in the other allele of the gene encoding the ANP32 protein. Animals, offspring, and cells that are heterozygous for the modified chromosomal sequence in the gene encoding the ANP32 protein include animals also include animals, offspring, and cells having a modified chromosomal sequence in one allele of a gene encoding the ANP32 protein and a different modified chromosomal sequence in the other allele of the gene encoding the ANP32 protein.

Alternatively, the animal, offspring, or cell can be homozygous for the modified chromosomal sequence in the gene encoding the ANP32 protein. Animals, offspring, or cells that are homozygous for the modified chromosomal sequence in the gene encoding the ANP32 protein have the same modified chromosomal sequence in both alleles of the gene.

The modified chromosomal sequence can comprise any alteration in the nucleotide sequence of the gene encoding the ANP32 protein. For example, the modified chromosomal sequence can comprise a substitution, an insertion, a frameshift, a deletion, an inversion, a translocation, a duplication, a splice-donor site alteration, or a combination of any thereof.

Thus, for example, in any of the animals, offspring, or cells, the modified chromosomal sequence can comprise a deletion in the gene encoding the ANP32 protein, an insertion in the gene encoding the ANP32 protein, a substitution in the gene encoding the ANP32 protein, or a combination of any thereof.

For example, the modified chromosomal sequence can comprise a deletion in the gene encoding the ANP32 protein.

The deletion can comprise an in-frame deletion.

The deletion can result in deletion of the entire coding sequence of the gene encoding the ANP32 protein.

The deletion can comprise a deletion of the start codon of the gene encoding the ANP32 protein.

The modified chromosomal sequence can comprise an insertion in the gene encoding the ANP32 protein.

The modified chromosomal sequence can comprise a substitution in the gene encoding the ANP32 protein.

The deletion, the insertion, and/or the substitution can result in a miscoding in the gene encoding the ANP32 protein.

Where the ungulate animal, offspring, or cell comprises a deletion in the gene encoding the ANP32 protein, an insertion in the gene encoding the ANP32 protein, and/or a substitution in the gene encoding the ANP32, the deletion, the insertion, and/or the substitution can result in introduction of a premature stop codon into the gene encoding the ANP32 protein. In some configurations, the edited site resulting in a premature stop codon can comprise SEQ ID NO: 7577 or SEQ ID NO: 7578.

In any of the animals, offspring, or cells, the modified chromosomal sequence in the gene encoding the ANP32 protein can comprise a modified chromosomal sequence in a gene encoding an ANP32A protein.

Where the animal, offspring, or cell comprises a modified chromosomal sequence in a gene encoding an ANP32A protein, the ANP32A protein can comprise an amino acid sequence having at least 80% sequence identity to SEQ ID NO: 6 or 7.

For example, the ANP32A protein can comprise an amino acid sequence having at least 85% sequence identity to SEQ ID NO: 6 or 7.

The ANP32A protein can comprise an amino acid sequence having at least 90% sequence identity to SEQ ID NO: 6 or 7.

The ANP32A protein can comprise an amino acid sequence having at least 95% sequence identity to SEQ ID NO: 6 or 7.

The ANP32A protein can comprise an amino acid sequence having at least 98% sequence identity to SEQ ID NO: 6 or 7.

The ANP32A protein can comprise an amino acid sequence having at least 99% sequence identity to SEQ ID NO: 6 or 7.

The ANP32A protein can comprise an amino acid sequence having at least 99.5% sequence identity to SEQ ID NO: 6 or 7.

For example, the ANP32A protein can comprise an amino acid sequence having at least 80%, at least 85%, at least 90%, at least 95%, at least 98%, at least 99%, or at least 99.5% sequence identity to SEQ ID NO: 6.

In any of the animals, offspring or cells, the modified chromosomal sequence in the gene encoding the ANP32A protein can result in introduction of a premature stop codon into the gene encoding the ANP32A protein. The premature stop codon can be upstream of the sequence in the gene encoding the ANP32A protein that codes for asparagine 129 (N129) and aspartic acid 130 (D130) of SEQ ID NO: 6 or asparagine 139 (N139) and aspartic acid 130 (D130) of SEQ ID NO: 7. The premature stop codon can be part of a sequence comprising SEQ ID NO: 7577.

In any of the animals, offspring or cells, the modified chromosomal sequence can comprise a modification in exon 1, exon 2, exon 3, or exon 4 of the gene encoding the ANP32A protein.

Alternatively or in addition, the modified chromosomal sequence can comprise a modification in an intron that is contiguous with any of exon 1, exon 2, exon 3, or exon 4 of the gene encoding the ANP32A protein.

For example, the modified chromosomal sequence can comprise a modification in exon 2 of the gene encoding the ANP32A protein, exon 3 of the gene encoding the ANP32A protein, or a sequence spanning an intron-exon junction between intron 1 and exon 2 of the gene encoding the ANP32A protein.

Alternatively or in addition, the modified chromosomal sequence can comprise a modification in exon 4 of the gene encoding the ANP32A protein.

In any of the animals, offspring or cells, the modified chromosomal sequence can comprise a modification within the region comprising nucleotides 29,400 through 31,996 of SEQ ID NO: 3.

For example, the modified chromosomal sequence can comprise a modification within the region comprising nucleotides 29,400 through 29,580 of SEQ ID NO: 3.

The modified chromosomal sequence can comprise a modification within the region comprising nucleotides 29,719 through 29,841 of SEQ ID NO: 3.

The modified chromosomal sequence can comprise a modification within the region comprising nucleotides 31,798 through 31,996 of SEQ ID NO: 3.

The modified chromosomal sequence can comprise a modification within the region comprising nucleotides 31,855 through 31,860 of SEQ ID NO: 3. Nucleotides 31,855 through 31,860 of SEQ ID NO: 3 encode N129 and D130 of the ANP32A protein of SEQ ID NO: 6.

For example, the modified chromosomal sequence can comprise a substitution of one or more nucleotides within the region comprising nucleotides 31,855 through 31,860 of SEQ ID NO: 3 with a different nucleotide, resulting in a codon that encodes a different amino acid.

Alternatively or in addition, the modification can result in an in-frame deletion of a codon within the region comprising nucleotides 31,855 through 31,860 of SEQ ID NO: 3.

In any of the animals, offspring, or cells, the modification can result in substitution of the asparagine at position 129 (N129) of SEQ ID NO: 6 or the asparagine at position 139 (N139) of SEQ ID NO: 7 with a different amino acid.

For example, the modification can result in substitution of the asparagine at position 129 (N129) of SEQ ID NO: 6 or the asparagine at position 139 (N139) of SEQ ID NO: 7 with a glycine (G), alanine (A), serine (S), threonine (T), cysteine (C), valine (V), leucine (L), isoleucine (I), methionine (M), proline (P), phenylalanine (F), tyrosine (Y), tryptophan (W), aspartic acid (D), glutamic acid (E), glutamine (Q), histidine (H), lysine (K), or arginine (R) residue.

The modification can result in substitution of the asparagine at position 129 (N129) of SEQ ID NO: 6 or the asparagine at position 139 (N139) of SEQ ID NO: 7 with a glycine (G), isoleucine (I), valine (V), proline (P), tryptophan (W), aspartic acid (D), glutamic acid (E), glutamine (Q), histidine (H), lysine (K), or arginine (R) residue.

For instance, the modification can result in substitution of the asparagine at position 129 (N129) of SEQ ID NO:6 or the asparagine at position 139 (N139) of SEQ ID NO: 7 with an isoleucine (I) residue.

In any of the animals, offspring, or cells, the modification can result in substitution of the aspartic acid at position 130 (D130) of SEQ ID NO: 6 or the aspartic acid at position 140 (D140) of SEQ ID NO: 7 with a different amino acid.

For example, the modification can result in substitution of the aspartic acid at position 130 (D130) of SEQ ID NO: 6 or the aspartic acid at position 140 (D140) of SEQ ID NO: 7 with a glycine (G), alanine (A), serine (S), threonine (T), cysteine (C), valine (V), leucine (L), isoleucine (I), methionine (M), proline (P), phenylalanine (F), tyrosine (Y), tryptophan (W), glutamic acid (E), asparagine (N), glutamine (Q), histidine (H), lysine (K), or arginine (R) residue.

The modification can result in substitution of the aspartic acid at position 130 (D130) of SEQ ID NO: 6 or the aspartic acid at position 140 (D140) of SEQ ID NO: 7 with an alanine (A), asparagine (N), valine (V), leucine (L), isoleucine (I), methionine (M), proline (P), phenylalanine (F), tyrosine (Y), tryptophan (W), glutamine (Q), histidine (H), lysine (K), or arginine (R) residue.

For instance, the modification can result in substitution of the aspartic acid at position 130 (D130) of SEQ ID NO: 6 or the aspartic acid at position 140 (D140) of SEQ ID NO: 7 with an alanine (A) or asparagine (N) residue.

In any of the animals, offspring, or cells, the modification can result in substitution of the asparagine at position 129 (N129) of SEQ ID NO: 6 or the asparagine at position 139 (N139) of SEQ ID NO: 7 with an isoleucine (I) residue and substitution of the aspartic acid at position 130 (D130) of SEQ ID NO: 6 or the aspartic acid at position 140 (D140) of SEQ ID NO: 7 with an alanine (A) or asparagine (N) residue.

In any of the animals, offspring or cells, the modified chromosomal sequence can comprise a modification in exon 3.

In any of the animals, offspring or cells, modified chromosomal sequence can comprise a modification within the region comprising nucleotides 29,830 through 29,832 of SEQ ID NO: 3. Nucleotides 29,830 through 29,832 of SEQ ID NO: 3 encode valine 106 (V106) of the ANP32A protein of SEQ ID NO: 6.

For example, the modified chromosomal sequence can comprise a substitution of one or more nucleotides within the region comprising nucleotides 29,830 through 29,832 of SEQ ID NO: 3 with a different nucleotide, resulting in a codon that encodes a different amino acid.

Alternatively, the modification can result in deletion of a codon at nucleotides 29,830 through 29,832 of SEQ ID NO: 3.

In any of the animals, offspring, or cells, the modification can result in substitution of the valine at position 106 (V106) of SEQ ID NO: 6 or the valine at position 116 of SEQ ID NO: 7 with a different amino acid.

For example, the modification can result in substitution of the valine at position 106 (V106) of SEQ ID NO: 6 or the valine at position 116 of SEQ ID NO: 7 with a glycine (G), alanine (A), serine (S), threonine (T), cysteine (C), leucine (L), isoleucine (I), methionine (M), proline (P), phenylalanine (F), tyrosine (Y), tryptophan (W), aspartic acid (D), glutamic acid (E), asparagine (N), glutamine (Q), histidine (H), lysine (K), or arginine (R) residue.

The modification can result in substitution of the valine at position 106 (V106) of SEQ ID NO: 6 or the valine at position 116 of SEQ ID NO: 7 with an isoleucine (I) residue.

In any of the animals, offspring, or cells, the modified chromosomal sequence can comprise a modification within the region comprising nucleotides 31,936 through 31,938 of SEQ ID NO: 3. Nucleotides 31,936 through 31,938 of SEQ ID NO: 3 encode serine 156 (S156) of the ANP32A protein of SEQ ID NO: 6.

For example, the modified chromosomal sequence can comprise a substitution of one or more nucleotides within the region comprising nucleotides 31,936 through 31,938 of SEQ ID NO: 3 with a different nucleotide, resulting in a codon that encodes a different amino acid.

Alternatively, the modification can result in deletion of a codon at nucleotides 31,936 through 31,938 of SEQ ID NO: 3.

In any of the animals, offspring, or cells, the modification can result in substitution of the serine at position 156 (S156) of SEQ ID NO: 6 or the serine at position 166 (S166) of SEQ ID NO: 7 with a different amino acid.

For example, the modification can result in substitution of the serine at position 156 (S156) of SEQ ID NO: 6 or the serine at position 166 (S166) of SEQ ID NO: 7 with a glycine (G), alanine (A), threonine (T), cysteine (C), valine (V), leucine (L), isoleucine (I), methionine (M), proline (P), phenylalanine (F), tyrosine (Y), tryptophan (W), aspartic acid (D), glutamic acid (E), asparagine (N), glutamine (Q), histidine (H), lysine (K), or arginine (R) residue.

The modification can result in substitution of the serine at position 156 (S156) of SEQ ID NO: 6 or the serine at position 166 (S166) of SEQ ID NO: 7 with a proline (P) residue.

In any of the animals, offspring, or cells, the modified chromosomal sequence in the gene encoding the ANP32 protein can comprise a modified chromosomal sequence in a gene encoding an ANP32B protein.

Where the animal, offspring, or cell comprises a modified chromosomal sequence in a gene encoding an ANP32B protein, the ANP32B protein can comprise an amino acid sequence having at least 80% sequence identity to any one of SEQ ID NOs. 8-14.

For example, the ANP32B protein can comprise an amino acid sequence having at least 90% sequence identity to any one of SEQ ID NOs. 8-14.

The ANP32B protein can comprise an amino acid sequence having at least 95% sequence identity to any one of SEQ ID NOs. 8-14.

The ANP32B protein can comprise an amino acid sequence having at least 98% sequence identity to any one of SEQ ID NOs. 8-14.

The ANP32B protein can comprise an amino acid sequence having at least 99% sequence identity to any one of SEQ ID NOs. 8-14.

The ANP32B protein can comprise an amino acid sequence having at least 99.5% sequence identity to any one of SEQ ID NOs. 8-14.

For example, the ANP32B protein ca n comprise an amino acid sequence having at least 80%, at least 90%, at least 95%, at least 98%, at least 99%, or at least 99.5% sequence identity to any one of SEQ ID NOs. 8-10.

In any of the animals, offspring, or cells, the modified chromosomal sequence in the gene encoding the ANP32B protein can result in introduction of a premature stop codon into the gene encoding the ANP32B protein. The premature stop codon can be upstream of the sequence in the gene encoding the ANP32B protein that codes for asparagine 129 (N129) and aspartic acid 130 (D130) of SEQ ID NO: 8, 9, or 10. The premature stop codon can be part of a sequence comprising SEQ ID NO: 7578.

In any of the animals, offspring, or cells, the modified chromosomal sequence can comprise a modification in exon 1, exon 2, exon 3, or exon 4 of the gene encoding the ANP32B protein.

Alternatively or in addition, the modified chromosomal sequence can comprise a modification in an intron that is contiguous with any of exon 1, exon 2, exon 3, or exon 4 of the gene encoding the ANP32B protein.

For example, the modified chromosomal sequence can comprise a modification in exon 2 of the gene encoding the ANP32B protein, exon 3 of the gene encoding the ANP32B protein, or a sequence spanning an intron-exon junction between exon 3 and intron 3 of the gene encoding the ANP32B protein.

Alternatively or in addition, the modified chromosomal sequence can comprise a modification in exon 4.

In any of the animals, offspring, or cells, the modified chromosomal sequence can comprise a modification within the region comprising nucleotides 10,823 through 20,342 of SEQ ID NO: 5.

For example, the modified chromosomal sequence can comprise a modification within the region comprising nucleotides 10,823-10,972 of SEQ ID NO: 5.

The modified chromosomal sequence can comprise a modification within the region comprising nucleotides 14,272-14,415 of SEQ ID NO: 5.

The modified chromosomal sequence can comprise a modification within the region comprising nucleotides 20,153-20,342 of SEQ ID NO: 5.

The modified chromosomal sequence can comprise a modification within the region comprising nucleotides 20,211 through 20,216 of SEQ ID NO: 5. Nucleotides through 20,216 of SEQ ID NO: 5 encode N129 and D130 of the ANP32B protein of SEQ ID NO: 8.

For example, the modified chromosomal sequence can comprise a substitution of one or more nucleotides within the region comprising nucleotides 20,211 through 20,216 of SEQ ID NO: 5 with a different nucleotide, resulting in a codon that encodes a different amino acid.

Alternatively or in addition, the modification can result in an in-frame deletion of a codon within the region comprising nucleotides 20,211 through 20,216 of SEQ ID NO: 5.

In any of the animals, offspring, or cells, the modification can result in substitution of the asparagine at position 129 (N129) of SEQ ID NO: 8, 9, or 10 with a different amino acid.

For example, the modification can result in substitution of the asparagine at position 129 (N129) of SEQ ID NO: 8, 9, or 10 with a glycine (G), alanine (A), serine (S), threonine (T), cysteine (C), valine (V), leucine (L), isoleucine (I), methionine (M), proline (P), phenylalanine (F), tyrosine (Y), tryptophan (W), aspartic acid (D), glutamic acid (E), glutamine (Q), histidine (H), lysine (K), or arginine (R) residue.

The modification can result in substitution of the asparagine at position 129 (N129) of SEQ ID NO: 8, 9, or 10 with a glycine (G), isoleucine (I), valine (V), proline (P), tryptophan (W), aspartic acid (D), glutamic acid (E), glutamine (Q), histidine (H), lysine (K), or arginine (R) residue.

The modification can result in substitution of the asparagine at position 129 (N129) of SEQ ID NO: 8, 9, or 10 with an isoleucine (I) residue.

In any of the animals, offspring, or cells, the modification can result in substitution of the aspartic acid at position 130 (D130) of SEQ ID NO: 8, 9, or 10 with a different amino acid.

For example, the modification can result in substitution of the aspartic acid at position 130 (D130) of SEQ ID NO: 8, 9, or 10 with a glycine (G), alanine (A), serine (S), threonine (T), cysteine (C), valine (V), leucine (L), isoleucine (I), methionine (M), proline (P), phenylalanine (F), tyrosine (Y), tryptophan (W), glutamic acid (E), asparagine (N), glutamine (Q), histidine (H), lysine (K), or arginine (R) residue.

The modification can result in substitution of the aspartic acid at position 130 (D130) of SEQ ID NO: 8, 9, or 10 with an alanine (A), asparagine (N), valine (V), leucine (L), isoleucine (I), methionine (M), proline (P), phenylalanine (F), tyrosine (Y), tryptophan (W), glutamine (Q), histidine (H), lysine (K), or arginine (R) residue.

The modification can result in substitution of the aspartic acid at position 130 (D130) of SEQ ID NO: 8, 9, or 10 with an alanine (A) or asparagine (N) residue.

In any of the animals, offspring, or cells, the modification can in substitution of the asparagine at position 129 (N129) of SEQ ID NO: 8, 9, or 10 with an isoleucine (I) residue and substitution of the aspartic acid at position 130 (D130) of SEQ ID NO: 8, 9, or 10 with an alanine (A) or asparagine (N) residue.

In any of the animals, offspring, or cells, the animal, offspring, or cell can have modified chromosomal sequences in both the gene encoding the ANP32A protein and the gene encoding the ANP32B protein. Thus, the at least one modified chromosomal sequence in the gene encoding the ANP32 protein can comprise a modified chromosomal sequence in a gene encoding an ANP32A protein and a modified chromosomal sequence in a gene encoding an ANP32B protein. The modified chromosomal sequences can comprise any of the modified chromosomal sequences described herein.

In any of the animals, offspring, or cells, the modified chromosomal sequence in the gene encoding the ANP32 protein preferably causes production or activity of the ANP32 protein to be reduced, as compared to the production or activity of the same ANP32 protein in an animal, offspring, or cell that lacks the modified chromosomal sequence in the gene encoding the ANP32 protein.

Preferably, the modified chromosomal sequence in the gene encoding the ANP32 protein results in production of substantially no functional ANP32A and/or ANP32B protein by the animal, offspring, or cell. By “substantially no functional ANP32A and/or ANP32B protein,” it is meant that the level of ANP32A and/or ANP32B protein in the animal, offspring, or cell is undetectable, or if detectable, is at least about 90% lower, preferably at least about 95% lower, more preferably at least about 98%, lower, and even more preferably at least about 99% lower than the level observed in an animal, offspring, or cell that does not comprise the modified chromosomal sequence.

For any of the animals, offspring, or cells described herein, the animal, offspring, or cell preferably does not produce ANP32A and/or ANP32B protein.

In any of the animals, offspring, or cells described herein, the modified chromosomal sequence can disrupt an intron-exon splice region. Disruption of an intron-exon splice region can result in exon skipping or intron inclusion due to lack of splicing downstream of the intron-exon splice region, as well as additional downstream exons in the resulting mRNA.

In order to disrupt an intron-exon splice region, any nucleotide that is required for splicing can be altered. For example, most introns end in the sequence “AG.” If the guanine (G) residue in this sequence is replaced with a different base, the splice will not occur at this site and will instead occur at the next downstream AG dinucleotide.

Intron-exon splice regions can also be disrupted by modifying the sequence at the beginning of the intron. Most introns begin with the consensus sequence RRGTRRRY, where “R” is any purine and “Y” is any pyrimidine. If the guanine (G) residue in this sequence is modified and/or if two or more of the other bases are modified, the intron can be rendered non-functional and will not splice.

Intron-exon splice regions can also be disrupted by any other methods known in the art.

In any of the animals, offspring, or cells described herein, the modified chromosomal sequence in the gene encoding the ANP32 protein can consist of the insertion, deletion, or substitution in the gene encoding the ANP32 protein.

In any of the animals, offspring, or cells described herein, the animal, offspring or cell can comprise a chromosomal sequence having at least 80% sequence identity to SEQ ID NO: 2, 3, 4, or 5 in the regions outside of the insertion, the deletion, or the substitution.

The animal, offspring or cell can comprise a chromosomal sequence having at least 85% sequence identity to SEQ ID NO: 2, 3, 4, or 5 in the regions outside of the insertion, the deletion, or the substitution.

The animal, offspring or cell can comprise a chromosomal sequence having at least 90% sequence identity to SEQ ID NO: 2, 3, 4, or 5 in the regions outside of the insertion, the deletion, or the substitution.

The animal, offspring or cell can comprise a chromosomal sequence having at least 95% sequence identity to SEQ ID NO: 2, 3, 4, or 5 in the regions outside of the insertion, the deletion, or the substitution.

The animal, offspring or cell can comprise a chromosomal sequence having at least 98% sequence identity to SEQ ID NO: 2, 3, 4, or 5 in the regions outside of the insertion, the deletion, or the substitution.

The animal, offspring or cell can comprise a chromosomal sequence having at least 99% sequence identity to SEQ ID NO: 2, 3, 4, or 5 in the regions outside of the insertion, the deletion, or the substitution.

The animal, offspring or cell can comprise a chromosomal sequence having at least 99.9% sequence identity to SEQ ID NO: 2, 3, 4, or 5 in the regions outside of the insertion, the deletion, or the substitution.

The animal, offspring or cell can comprise a chromosomal sequence having 100% sequence identity to SEQ ID NO: 2, 3, 4, or 5 in the regions outside of the insertion, the deletion, or the substitution.

### Genetically Edited Animals and Cells

Any of the animals or offspring described herein can be genetically edited animals or offspring.

Likewise, any of the cells described herein can be genetically edited cells.

The animal, offspring, or cell can be an animal, offspring, or cell that has been genetically edited using a homing endonuclease. The homing endonuclease can be a naturally occurring endonuclease but is preferably a rationally designed, non-naturally occurring homing endonuclease that has a DNA recognition sequence that has been designed so that the endonuclease targets a chromosomal sequence in a gene encoding an ANP32 protein. Thus, the homing endonuclease can be a designed homing endonuclease.

The homing endonuclease can comprise, for example, a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

The homing nuclease preferably comprises a CRISPR system. Examples of CRISPR systems that can be used to create the female porcine animals for use in the methods described herein include, but are not limited to CRISPR/Cas9, CRISPR/Cas5, CRISPR/Cas6, and CRISPR/Cas12.

The use of various homing endonucleases, including CRISPR systems and TALENs, to generate genetically edited animals is discussed further hereinbelow.

The edited chromosomal sequence may be (1) inactivated, (2) modified, or (3) comprise an integrated sequence. Where the edited chromosomal sequence is in an ANP32 gene, a chromosomal sequence is altered such that an ANP32 protein function as it relates to type A influenza infection is impaired, reduced, or eliminated. Thus, a genetically edited animal comprising an inactivated chromosomal sequence may be termed a “knock out” or a “conditional knock out.” Similarly, a genetically edited animal comprising an integrated sequence may be termed a “knock in” or a “conditional knock in.” Furthermore, a genetically edited animal comprising a modified chromosomal sequence may comprise one or more targeted base pair alteration(s) or other modification(s) such that an altered protein product is produced. Briefly, the process can comprise introducing into an embryo or cell at least one RNA molecule encoding a targeted zinc finger nuclease and, optionally, at least one accessory polynucleotide. The process further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The process of editing chromosomal sequences encoding a protein associated with germline development using targeted zinc finger nuclease technology is rapid, precise, and highly efficient.

Alternatively, the process can comprise using a CRISPR system (e.g., a CRISPR/Cas9 system) to modify the genomic sequence. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target-specific crRNA and a tracrRNA can be delivered directly to a cell or target-specific sgRNA(s) can be to a cell (these RNAs can alternatively be transcribed from a plasmid constructed to encode the RNAs). Selection of target sites and designed of crRNA/gRNA are well known in the art. A discussion of construction and cloning of gRNAs can be found at http://www(dot)genome-engineering(dot)org/crispr/wp-content/uploads/2014/05/CRISPR-Reagent-Description-Rev20140509.pdf.

At least one ANP32 locus can be used as a target site for the site-specific editing. The site-specific editing can include insertion of an exogenous nucleic acid (e.g., a nucleic acid comprising a nucleotide sequence encoding a polypeptide of interest) or deletions of nucleic acids from the locus. For example, integration of the exogenous nucleic acid and/or deletion of part of the genomic nucleic acid can modify the locus so as to produce a disrupted ANP32 gene, resulting in reduced activity of ANP32 protein.

Thus, for example, any of the animals or cells can be an animal or cell that has been genetically edited using a Clustered Regularly Interspaced Short Palindromic Repeats (CRISPR)/Cas system. The CRISPR/Cas system can suitably comprise any of the guide RNAs (gRNAs) described herein.

For any of the animals, offspring, or cells, the modified chromosomal sequence can be a modified chromosomal sequence that has been produced via homology directed repair (HDR).

Alternatively, the modified chromosomal sequence can be a modified chromosomal sequence that has been produced via non-homologous end-joining (NEHJ). For example, it is advantageous to identify and use guide pairs that result in the deletion of DNA sequences such that the joined ends can result in the generation of an in-frame translational stop codon across the joined ends; when the cut sites of two guides are repaired by NHEJ in an end-to-end manner, this new DNA sequence, when transcribed into mRNA and translated into protein could terminate the production of the ANP32 protein.

### Cell Types

Any of the cells described herein can comprise a germ cell or a gamete.

For example, any of the cells described herein can comprise a sperm cell.

Alternatively, any of the cells described herein can comprise an egg cell (e.g., a fertilized egg).

Any of the cells described herein can comprise a somatic cell.

For example, any of the cells described herein can comprise a fibroblast (e.g., a fetal fibroblast).

Any of the cells described herein can comprise an embryonic cell.

Any of the cells described herein can comprise a cell derived from a juvenile animal.

Any of the cells described herein can comprise a cell derived from an adult animal.

### Methods for Producing Animals

Methods for producing pathogen-resistant ungulate animals and lineages of such animals are provided.

The methods can comprise introducing into an animal cell or an oocyte or embryo an agent that specifically binds to a chromosomal target site of the cell and causes a double-stranded DNA break or otherwise inactivates or reduces activity of an ANP32 gene or protein therein using gene editing methods such as the Clustered Regularly Interspaced Short Palindromic Repeats (CRISPR)/Cas system, Transcription Activator-Like Effector Nucleases (TALENs), Zinc Finger Nucleases (ZFN), recombinase fusion proteins, or meganucleases.

Also described herein is the use of one or more particular ANP32 loci in tandem with a polypeptide capable of effecting cleavage and/or integration of specific nucleic acid sequences within the one or more ANP32 loci. Examples of the use of ANP32 loci in tandem with a polypeptide or RNA capable of effecting cleavage and/or integration of the ANP32 loci include a polypeptide selected from the group consisting of zinc finger proteins, meganucleases, TAL domains, TALENs, RNA-guided CRISPR/Cas recombinases, leucine zippers, and others known to those in the art. Particular examples include a chimeric (“fusion”) protein comprising a site-specific DNA binding domain polypeptide and cleavage domain polypeptide (e.g., a nuclease), such as a ZFN protein comprising a zinc-finger polypeptide and a FokI nuclease polypeptide. Described herein are polypeptides comprising a DNA-binding domain that specifically binds to an ANP32 gene. Such a polypeptide can also comprise a nuclease (cleavage) domain or half-domain (e.g., a homing endonuclease, including a homing endonuclease with a modified DNA-binding domain), and/or a ligase domain, such that the polypeptide may induce a targeted double-stranded break, and/or facilitate recombination of a nucleic acid of interest at the site of the break. A DNA-binding domain that targets an ANP32 locus can be a DNA-cleaving functional domain. The foregoing polypeptides can be used to introduce an exogenous nucleic acid into the genome of a host organism (e.g., an animal species) at one or more ANP32 loci. The DNA-binding domains can comprise a zinc finger protein with one or more zinc fingers (e.g., 2, 3, 4, 5, 6, 7, 8, 9 or more zinc fingers), which is engineered (non-naturally occurring) to bind to any sequence within an ANP32 gene. Any of the zinc finger proteins described herein may bind to a target site within the coding sequence of the target gene or within adjacent sequences (e.g., promoter or other expression elements). The zinc finger protein can bind to a target site in an ANP32 gene.

A method for producing an ungulate animal or a lineage of ungulate animals having reduced susceptibility to infection by a pathogen is provided. The method comprises modifying an ungulate oocyte or an ungulate sperm cell to introduce a modified chromosomal sequence in a gene encoding an ANP32 protein into at least one of the oocyte and the sperm cell, and fertilizing the oocyte with the sperm cell to create a fertilized egg containing the modified chromosomal sequence in the gene encoding the ANP32 protein. The method further comprises transferring the fertilized egg into a surrogate female ungulate animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANP32 protein.

Artificial insemination can be used to fertilize the oocyte with the sperm cell.

Another method for producing an ungulate animal or a lineage of ungulate animals having reduced susceptibility to infection by a pathogen is provided. The method comprises modifying an ungulate fertilized egg to introduce a modified chromosomal sequence in a gene encoding an ANP32 protein into the fertilized egg. The method further comprises transferring the fertilized egg into a surrogate female ungulate animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANP32 protein.

Yet another method for producing an ungulate animal or a lineage of ungulate animals having reduced susceptibility to infection by a pathogen is provided. The method comprises enucleating an ungulate oocyte, modifying a donor ungulate somatic cell to introduce a modified chromosomal sequence into a gene encoding an ANP32 protein, fusing the oocyte with the modified donor ungulate somatic cell, and activating the oocyte to produce an embryo. The method further comprises transferring the embryo into a surrogate female ungulate animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANP32 protein.

The donor ungulate somatic cell can comprise a fibroblast (e.g., a fetal fibroblast).

A method of increasing an ungulate animal's resistance to infection with a pathogen is provided. The method comprises modifying at least one chromosomal sequence in at least one gene encoding an ANP32 protein, so that production or activity of the ANP32 protein is reduced, as compared to production or activity of the same ANP32 protein in an ungulate animal that does not comprise a modified chromosomal sequence in the gene encoding the ANP32 protein.

In any of the methods, the oocyte, sperm cell, fertilized egg, donor somatic cell, or ungulate animal can be heterozygous for the modified chromosomal sequence.

Alternatively, the oocyte, sperm cell, fertilized egg, donor somatic cell, or ungulate animal can be homozygous for the modified chromosomal sequence.

In any of the methods, the step of modifying the at least one chromosomal sequence in the gene encoding the ANP32 protein can comprise genetic editing of the chromosomal sequence.

The genetic editing can comprise use of a homing endonuclease. The homing endonuclease can be a naturally occurring endonuclease but is preferably a rationally designed, non-naturally occurring homing endonuclease that has a DNA recognition sequence that has been designed so that the endonuclease targets a chromosomal sequence in a gene encoding an ANP32 protein. Thus, the homing endonuclease can be a designed homing endonuclease.

The homing endonuclease can comprise, for example, a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

The homing nuclease preferably comprises a CRISPR/Cas system. Examples of CRISPR systems that include, but are not limited to CRISPR/Cas9, CRISPR/Cas5, CRISPR/Cas6, and CRISPR/Cas12.

The CRISPR/Cas system can comprise a gRNA comprising a sequence that is complementary to a sequence of a gene encoding an ANP32A protein.

For example, the gRNA can comprise a sequence that is complementary to: (1) a sequence within exon 1, exon 2, or exon 3, or exon 4 of the gene encoding the ANP32A protein; (2) a sequence within an intron that is contiguous with any of exon 1, exon 2, exon 3, and exon 4 of the gene encoding the ANP32A protein; or (3) a sequence spanning an intron-exon junction between any of exon 1, exon 2, exon 3, and exon 4 and a contiguous intron.

Illustrative nucleic acid sequences for gRNAs that are complementary to a sequence of a gene encoding an ANP32A protein provided hereinbelow in Table 6 (SEQ ID NOs. 15-41). Additional nucleic acid sequences for gRNAs that are complementary to a sequence of a gene encoding an ANP32A protein are provided in the sequence listing as SEQ ID NOs. 61-5,109.

Thus, where the CRISPR/Cas9 system comprises a gRNA comprising a sequence that is complementary to a sequence of a gene encoding an ANP32A protein, the nucleic acid sequence of the gRNA can comprise any one of SEQ ID NOs. 15-41 and 61-5,109. For example, the nucleic acid sequence of the gRNA can comprise any one of SEQ ID NOs. 15-41. The sequence can also comprise a pair of gRNAs targeting ANP32A. For example, SEQ ID NOs: 26 and 19, SEQ ID NOs: 33 and 36, SEQ ID NOs: 33 and 40, SEQ ID NOs: 15 and 22, SEQ ID NOs: 15 and 23, SEQ ID NOs: 16 and 24, and SEQ ID NOs: 17 and 24.

The CRISPR/Cas system can comprise a gRNA comprising a sequence that is complementary to a sequence of a gene encoding an ANP32B protein.

For example, the gRNA can comprise a sequence that is complementary to: (1) a sequence within exon 1, exon 2, exon 3, or exon 4 of the gene encoding the ANP32B protein; (2) a sequence within an intron that is contiguous with any of exon 1, exon 2, exon 3, and exon 4 of the gene encoding the ANP32B protein; or (3) a sequence spanning an intron-exon junction between any of exon 1, exon 2, exon 3, and exon 4 and a contiguous intron.

Illustrative nucleic acid sequences for gRNAs that are complementary to a sequence of a gene encoding an ANP32A protein provided hereinbelow in Table 7 (SEQ ID NOs. 42-60). Additional nucleic acid sequences for gRNAs that are complementary to a sequence of a gene encoding an ANP32B protein are provided in the sequence listing as SEQ ID NOs. 5,110-7,576.

Thus, where the CRISPR/Cas9 system comprises a gRNA comprising a sequence that is complementary to a sequence of a gene encoding an ANP32B protein, the nucleic acid sequence of the gRNA can comprise any one of SEQ ID NOs. 42-60 and 5,110-7,576. For example, the nucleic acid sequence of the gRNA can comprise any one of SEQ ID NOs. 42-60.

Alternatively or in addition, the CRISPR/Cas9 system can comprise a pair of gRNAs targeted to an ANP32B gene. For example, the pair of gRNAs can comprise SEQ ID NOs: 44 and 46, SEQ ID NOs: 55 and 58, or SEQ ID NOs: 55 and 59.

In any of the methods, involving the use of a CRISPR/Cas system, the sequence of the gene encoding the ANP32A protein or the sequence of the gene encoding the ANP32B protein suitably comprises a protospacer adjacent motif (PAM). The PAM can comprise the sequence nGG, wherein n is any nucleotide.

In any of the methods, involving the use of a CRISPR/Cas system, the CRISPR/Cas system can comprise a Cas9 nuclease (e.g., a Streptococcus pyogenes Cas9 endonuclease).

In any of the methods described herein, the ungulate animal can comprise a porcine animal or a bovine animal. For example, the ungulate animal can comprise a porcine animal.

Any of the methods described herein can produce any of the animals described herein or any of the ungulate cells described herein.

Any of the methods described herein can further comprise using the animal as a founder animal.

### Populations of Animals

Populations of animals described herein are also provided.

A population of ungulate animals is provided. The population comprises two or more of any of the ungulate animals and/or offspring thereof described herein.

Another population of animals is provided. The population comprises two or more ungulate animals made by any of the methods described herein, and/or offspring thereof.

The populations of animals are resistant to infection by a pathogen.

The pathogen can comprise a virus. For example, the pathogen can comprise an influenza virus (e.g., a type A influenza virus).

### Guide RNAs

Guide RNAs (gRNAs) are provided. The gRNAs have a nucleic acid sequence that is complementary to a sequence of a gene encoding an ANP32 protein and can be used to introduce a chromosomal modification into a gene encoding an ANP32 protein.

Illustrative gRNA sequences complementary to a sequence of a gene encoding an ANP32A protein or a gene encoding an ANP32B protein are provided in Tables 6 and 7 below. The numbering and strandedness of the gRNA sequences provided in Tables 6 and 7 correspond to the numbering and strandedness of SEQ ID NO: 3 (for the ANP32A gRNAs provided in Table 6) and SEQ ID NO: 5 (for the ANP32B gRNAs provided in Table 7). A “1” in the “strand” column in Tables 6 and 7 indicates that the sequence of the gRNA is a sequence that is found on the same strand as the strand for which the nucleic acid sequence is provided in SEQ ID NO: 3 or 5, whereas a “−1” in the “strand” column indicates that the sequence of the gRNA is on the opposing strand. The “position” listed in Tables 6 and 7 is the predicted nuclease cut site, according to the nucleotide numbering of SEQ ID NOs. 3 and 5.

Tables 6 and 7 also provide the PAM sequence found in the sequence of the gene encoding the ANP32 protein. Although the sequences shown in Table 6 and 7 are listed with DNA nucleotides, a person of ordinary skill would understand that the sequences are in fact RNA sequences and would be readily able to convert the DNA sequences into RNA sequences.

TABLE 6
Illustrative ANP32A gRNA sequences

| SEQ ID NO. | Position | Strand | Sequence | PAM | Location |
|---|---|---|---|---|---|
| 15 | 29419 | −1 | ttcacctggaaagacaaggc | cgg | intron 1 |
| 16 | 29423 | −1 | ctctttcacctggaaagaca | agg | intron 1 |
| 17 | 29426 | 1 | cacgccggccttgtctttcc | agg | intron 1 |
| 18 | 29433 | −1 | ccaggacgagctctttcacc | tgg | exon 2 |
| 19 | 29444 | 1 | ccaggtgaaagagctcgtcc | tgg | exon 2 |
| 20 | 29451 | −1 | catttgaccgacaattgtcc | agg | exon 2 |
| 21 | 29455 | 1 | agctcgtcctggacaattgt | cgg | exon 2 |
| 22 | 29466 | 1 | gacaattgtcggtcaaatga | agg | exon 2 |
| 23 | 29478 | 1 | tcaaatgaaggcaagattga | agg | exon 2 |
| 24 | 29490 | −1 | gttcttcaaattcatctgtg | agg | exon 2 |
| 25 | 29504 | 1 | cacagatgaatttgaagaac | tgg | exon 2 |
| 26 | 29529 | 1 | ttcttgagtacaatcaatgt | agg | exon 2 |
| 27 | 29541 | −1 | gtaagtttgcgactgaggtg | agg | exon 2 |
| 28 | 29546 | −1 | ctttggtaagtttgcgactg | agg | exon 2 |
| 29 | 29563 | −1 | ttcttaagtttgtttaactt | tgg | exon 2 |
| 30 | 29576 | 1 | aaagttaaacaaacttaaga | agg | exon 2 |
| 31 | 29742 | 1 | ctaagtgataacagaatctc | agg | exon 3 |
| 32 | 29743 | 1 | taagtgataacagaatctca | ggg | exon 3 |
| 33 | 29744 | 1 | aagtgataacagaatctcag | ggg | exon 3 |
| 34 | 29745 | 1 | agtgataacagaatctcagg | ggg | exon 3 |
| 35 | 29750 | 1 | taacagaatctcagggggcc | tgg | exon 3 |
| 36 | 29757 | −1 | acttttctgccaatacttcc | agg | exon 3 |
| 37 | 29759 | 1 | ctcagggggcctggaagtat | tgg | exon 3 |
| 38 | 29782 | −1 | aaatttagatgtgtgaggtt | cgg | exon 3 |
| 39 | 29787 | −1 | cacttaaatttagatgtgtg | agg | exon 3 |
| 40 | 29799 | 1 | ctcacacatctaaatttaag | tgg | exon 3 |
| 41 | 29837 | 1 | cctcagcacagtagagccac | tgg | exon 3 |

TABLE 7
Illustrative ANP32B gRNA sequences

| SEQ ID NO. | Position | Strand | Sequence | PAM | Location |
|---|---|---|---|---|---|
| 42 | 10858 | 1 | gataattgcaaatcaagtga | tgg | exon 2 |
| 43 | 10859 | 1 | ataattgcaaatcaagtgat | ggg | exon 2 |
| 44 | 10869 | 1 | atcaagtgatgggaaaattg | agg | exon 2 |
| 45 | 10870 | 1 | tcaagtgatgggaaaattga | ggg | exon 2 |
| 46 | 10912 | −1 | accctacatttattaaactg | agg | exon 2 |
| 47 | 10921 | 1 | ttcctcagtttaataaatgt | agg | exon 2 |
| 48 | 10922 | 1 | tcctcagtttaataaatgta | ggg | exon 2 |
| 49 | 10954 | −1 | ttttcaatttaggtagtttg | ggg | exon 2 |
| 50 | 10955 | −1 | tttttcaatttaggtagttt | ggg | exon 2 |
| 51 | 10956 | −1 | ctttttcaatttaggtagtt | tgg | exon 2 |
| 52 | 10964 | −1 | gcacttacctttttcaattt | agg | exon 2 |
| 53 | 10968 | 1 | caaactacctaaattgaaaa | agg | exon 2 |
| 54 | 14295 | 1 | ctcagtgacaatagaatcta | tgg | exon 3 |
| 55 | 14298 | 1 | agtgacaatagaatctatgg | agg | exon 3 |
| 56 | 14303 | 1 | caatagaatctatggaggtc | tgg | exon 3 |
| 57 | 14335 | −1 | aagtttagatgtgtgagatt | tgg | exon 3 |
| 58 | 14387 | −1 | actacttaccaaaggttcca | ggg | exon 3 |
| 59 | 14388 | −1 | aactacttaccaaaggttcc | agg | exon 3 |
| 60 | 14395 | −1 | aattctcaactacttaccaa | agg | intron 3 |

The gRNA can comprise a nucleotide sequence comprising any one of SEQ ID NOs. 15-7,576.

For targeting an ANP32A gene, the gRNA can comprise a nucleotide sequence comprising any one of SEQ ID NOs. 15-41 and 61-5,109. For example, the gRNA can comprise any one of SEQ ID NOs. 15-41. Alternatively, the targeting molecules can comprise a pair of guides selected from any two of SEQ ID NOs: 15-41 and 61-5,109. The pair of guides can be selected from any two of SEQ ID NOs: 15-41. The pair of guides can be SEQ ID NOs: 26 and 19, SEQ ID NOs: 33 and 36, SEQ ID NOs: 33 and 40, SEQ ID NOs: 15 and 22, SEQ ID NOs: 15 and 23, SEQ ID NOs: 16 and 24, or SEQ ID NOs: 17 and 24.

For targeting an ANP32B gene, the gRNA can comprise a nucleotide sequence comprising any one of SEQ ID NOs. 42-60 and 5,110-7,576. For example, the gRNA can comprise any one of SEQ ID NOs. 42-60. Alternatively, the targeting molecules can comprise a pair of guides selected from any two of SEQ ID NOs: 42-60 and 5,110-7,576. The pair of guides can be selected from any two of SEQ ID NOs: 42-60. The pair of guides can be SEQ ID NOs: 44 and 46, SEQ ID NOs: 55 and 58, or SEQ ID NOs: 55 and 59.

The gRNA can have a length of 100 nucleotides or fewer, 90 nucleotides or fewer, 80 nucleotides or fewer, 70 nucleotides or fewer, 60 nucleotides or fewer, 50 nucleotides or fewer, 40 nucleotides or fewer, 30 nucleotides or fewer, or 20 nucleotides or fewer. For example, the gRNA can have a length of 20 nucleotides.

An exemplary edited sequence for ANP32A is gggaactggcaggcctcccgggcatagcccctcctgcgctctatttaccncttaagffigtttaactttggtaagtttgcgactgaggtga ggcctacacgagctattcacctggaaagacaaggccggcgtgaatggggtgaggaatggggcccaagaccggggaggggacag gaggcagaccagaaggcacctcta (SEQ ID NO: 7577). This sequence provides end-to-end cut site repair (NHEJ) for SEQ ID NOs: 26 and 19; the exogenous stop codon is created through this NHEJ repair. 100 bp on either end of the cut site are provided.

An exemplary edited sequence for ANP32B is ggttacttctaacaccttaatgatatgtgtttcttffigtgffigtgtgccgtgtgcatitticcctttaacagcttgagctcagtgacaatagaat ctaaccffiggtaagtagttgagaatttggaaaacaggactttctggicattncattncatatttctnatggtgagggaaataattgaaagtt ataatgg (SEQ ID NO: 7578). This sequence provides end-to-end cut site repair (NHEJ) for SEQ ID NOs: 55 and 59; the exogenous stop codon is created through this NHEJ repair. 100 bp on either end of the cut site are provided.

### Affinity Tags

An “affinity tag” can be either a peptide affinity tag or a nucleic acid affinity tag. The term “affinity tag” generally refers to a protein or nucleic acid sequence that can be bound to a molecule (e.g., bound by a small molecule, protein, or covalent bond). An affinity tag can be a non-native sequence. A peptide affinity tag can comprise a peptide. A peptide affinity tag can be one that is able to be part of a split system (e.g., two inactive peptide fragments can combine together in trans to form an active affinity tag). A nucleic acid affinity tag can comprise a nucleic acid. A nucleic acid affinity tag can be a sequence that can selectively bind to a known nucleic acid sequence (e.g. through hybridization). A nucleic acid affinity tag can be a sequence that can selectively bind to a protein. An affinity tag can be fused to a native protein. An affinity tag can be fused to a nucleotide sequence.

Sometimes, one, two, or a plurality of affinity tags can be fused to a native protein or nucleotide sequence. An affinity tag can be introduced into a nucleic acid-targeting nucleic acid using methods of in vitro or in vivo transcription. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, or a synthetic DNA aptamer. Examples of chemical nucleic acid affinity tags can include, but are not limited to, ribo-nucleotriphosphates containing biotin, fluorescent dyes, and digoxeginin. Examples of protein-binding nucleic acid affinity tags can include, but are not limited to, the MS2 binding sequence, the U1A binding sequence, stem-loop binding protein sequences, the boxB sequence, the eIF4A sequence, or any sequence recognized by an RNA binding protein. Examples of nucleic acid affinity-tagged oligonucleotides can include, but are not limited to, biotinylated oligonucleotides, 2,4-dinitrophenyl oligonucleotides, fluorescein oligonucleotides, and primary amine-conjugated oligonucleotides.

A nucleic acid affinity tag can be an RNA aptamer. Aptamers can include, aptamers that bind to theophylline, streptavidin, dextran B512, adenosine, guanosine, guanine/xanthine, 7-methyl-GTP, amino acid aptamers such as aptamers that bind to arginine, citrulline, valine, tryptophan, cyanocobalamine, N-methylmesoporphyrin IX, flavin, NAD, and antibiotic aptamers such as aptamers that bind to tobramycin, neomycin, lividomycin, kanamycin, streptomycin, viomycin, and chloramphenicol.

A nucleic acid affinity tag can comprise an RNA sequence that can be bound by a site-directed polypeptide. The site-directed polypeptide can be conditionally enzymatically inactive. The RNA sequence can comprise a sequence that can be bound by a member of Type I, Type II, and/or Type III CRISPR systems. The RNA sequence can be bound by a RAMP family member protein. The RNA sequence can be bound by a Cas9 family member protein, a Cas6 family member protein (e.g., Csy4, Cas6). The RNA sequence can be bound by a Cas5 family member protein (e.g., Cas5). For example, Csy4 can bind to a specific RNA hairpin sequence with high affinity (Kd ˜50 pM) and can cleave RNA at a site 3′ to the hairpin.

A nucleic acid affinity tag can comprise a DNA sequence that can be bound by a site-directed polypeptide. The site-directed polypeptide can be conditionally enzymatically inactive. The DNA sequence can comprise a sequence that can be bound by a member of the Type I, Type II, and/or Type III CRISPR systems. The DNA sequence can be bound by an Argonaut protein. The DNA sequence can be bound by a protein containing a zinc finger domain, a TALE domain, or any other DNA-binding domain.

A nucleic acid affinity tag can comprise a ribozyme sequence. Suitable ribozymes can include peptidyl transferase 23 SrRNA, RnaseP, Group I introns, Group II introns, GIR1 branching ribozyme, Leadzyme, hairpin ribozymes, hammerhead ribozymes, HDV ribozymes, CPEB3 ribozymes, VS ribozymes, glmS ribozyme, CoTC ribozyme, and synthetic ribozymes.

Peptide affinity tags can comprise tags that can be used for tracking or purification (e.g., a fluorescent protein such as green fluorescent protein (GFP), YFP, RFP, CFP, mCherry, tdTomato; a His tag, (e.g., a 6×His tag); a hemagglutinin (HA) tag; a FLAG tag; a Myc tag; a GST tag; a MBP tag; a chitin binding protein tag; a calmodulin tag; a V5 tag; a streptavidin binding tag; and the like).

Both nucleic acid and peptide affinity tags can comprise small molecule tags such as biotin, or digitoxin, and fluorescent label tags, such as for example, fluoroscein, rhodamin, Alexa fluor dyes, Cyanine3 dye, Cyanine5 dye.

Nucleic acid affinity tags can be located 5′ to a nucleic acid (e.g., a nucleic acid-targeting nucleic acid). Nucleic acid affinity tags can be located 3′ to a nucleic acid. Nucleic acid affinity tags can be located 5′ and 3′ to a nucleic acid. Nucleic acid affinity tags can be located within a nucleic acid. Peptide affinity tags can be located N-terminal to a polypeptide sequence. Peptide affinity tags can be located C-terminal to a polypeptide sequence. Peptide affinity tags can be located N-terminal and C-terminal to a polypeptide sequence. A plurality of affinity tags can be fused to a nucleic acid and/or a polypeptide sequence.

### Capture Agents

As used herein, “capture agent” can generally refer to an agent that can purify a polypeptide and/or a nucleic acid. A capture agent can be a biologically active molecule or material (e.g. any biological substance found in nature or synthetic, and includes but is not limited to cells, viruses, subcellular particles, proteins, including more specifically antibodies, immunoglobulins, antigens, lipoproteins, glycoproteins, peptides, polypeptides, protein complexes, (strept)avidin-biotin complexes, ligands, receptors, or small molecules, aptamers, nucleic acids, DNA, RNA, peptidic nucleic acids, oligosaccharides, polysaccharides, lipopolysaccharides, cellular metabolites, haptens, pharmacologically active substances, alkaloids, steroids, vitamins, amino acids, and sugars). In some embodiments, the capture agent can comprise an affinity tag. In some embodiments, a capture agent can preferentially bind to a target polypeptide or nucleic acid of interest. Capture agents can be free floating in a mixture. Capture agents can be bound to a particle (e.g. a bead, a microbead, a nanoparticle). Capture agents can be bound to a solid or semisolid surface. In some instances, capture agents are irreversibly bound to a target. In other instances, capture agents are reversibly bound to a target (e.g., if a target can be eluted, or by use of a chemical such as imidazole).

### Targeted Integration of a Nucleic Acid at an ANP32 Locus

Site-specific integration of an exogenous nucleic acid at an ANP32 locus may be accomplished by any technique known to those of skill in the art. For example, integration of an exogenous nucleic acid at an ANP32 locus can comprise contacting a cell (e.g., an isolated cell or a cell in a tissue or organism) with a nucleic acid molecule comprising the exogenous nucleic acid. Such a nucleic acid molecule can comprise nucleotide sequences flanking the exogenous nucleic acid that facilitate homologous recombination between the nucleic acid molecule and at least one ANP32 locus. The nucleotide sequences flanking the exogenous nucleic acid that facilitate homologous recombination can be complementary to endogenous nucleotides of the ANP32 locus. Alternatively, the nucleotide sequences flanking the exogenous nucleic acid that facilitate homologous recombination can be complementary to previously integrated exogenous nucleotides. A plurality of exogenous nucleic acids can be integrated at one ANP32 locus, such as in gene stacking.

Integration of a nucleic acid at an ANP32 locus can be facilitated (e.g., catalyzed) by endogenous cellular machinery of a host cell, such as, for example and without limitation, endogenous DNA and endogenous recombinase enzymes. Alternatively, integration of a nucleic acid at an ANP32 locus can be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, a nucleic acid comprising a nucleotide sequence encoding at least one nuclease, recombinase, and/or ligase polypeptide may be introduced into the host cell, either concurrently or sequentially with a nucleic acid to be integrated site-specifically at an ANP32 locus, wherein the at least one nuclease, recombinase, and/or ligase polypeptide is expressed from the nucleotide sequence in the host cell.

### DNA-Binding Polypeptides

Site-specific integration can be accomplished by using factors that are capable of recognizing and binding to particular nucleotide sequences, for example, in the genome of a host organism. For instance, many proteins comprise polypeptide domains that are capable of recognizing and binding to DNA in a site-specific manner. A DNA sequence that is recognized by a DNA-binding polypeptide may be referred to as a “target” sequence. Polypeptide domains that are capable of recognizing and binding to DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; Tet repressors; Lad; and steroid hormone receptors.

For example, the DNA-binding polypeptide can be a zinc finger. Individual zinc finger motifs can be designed to target and bind specifically to any of a large range of DNA sites. Canonical Cys2His2 (as well as non-canonical Cys3His) zinc finger polypeptides bind DNA by inserting an α-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Umov et al. (2005) Nature 435:646-51. Thus, one or more zinc finger DNA-binding polypeptides may be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261.

Illustrative selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for illustrative linker sequences 6 or more amino acids in length. The proteins described herein may include any combination of suitable linkers between the individual zinc fingers of the protein.

Selection of target sites: ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

Where an animal or cell as described herein has been genetically edited using a zinc-finger nuclease, the animal or cell can be created using a process comprising introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with germline development using targeted zinc finger nuclease technology is rapid, precise, and highly efficient.

Alternatively, the DNA-binding polypeptide is a DNA-binding domain from GAL4. GAL4 is a modular transactivator in Saccharomyces cerevisiae, but it also operates as a transactivator in many other organisms. See, e.g., Sadowski et al. (1988) Nature 335:563-4. In this regulatory system, the expression of genes encoding enzymes of the galactose metabolic pathway in S. cerevisiae is stringently regulated by the available carbon source. Johnston (1987) Microbiol. Rev. 51:458-76. Transcriptional control of these metabolic enzymes is mediated by the interaction between the positive regulatory protein, GAL4, and a 17 bp symmetrical DNA sequence to which GAL4 specifically binds (the upstream activation sequence (UAS)).

Native GAL4 consists of 881 amino acid residues, with a molecular weight of 99 kDa. GAL4 comprises functionally autonomous domains, the combined activities of which account for activity of GAL4 in vivo. Ma and Ptashne (1987) Cell 48:847-53); Brent and Ptashne (1985) Cell 43(3 Pt 2):729-36. The N-terminal 65 amino acids of GAL4 comprise the GAL4 DNA-binding domain. Keegan et al. (1986) Science 231:699-704; Johnston (1987) Nature 328:353-5. Sequence-specific binding requires the presence of a divalent cation coordinated by six Cys residues present in the DNA binding domain. The coordinated cation-containing domain interacts with and recognizes a conserved CCG triplet at each end of the 17 bp UAS via direct contacts with the major groove of the DNA helix. Marmorstein et al. (1992) Nature 356:408-14. The DNA-binding function of the protein positions C-terminal transcriptional activating domains in the vicinity of the promoter, such that the activating domains can direct transcription.

Additional DNA-binding polypeptides that can be used include, for example and without limitation, a binding sequence from a AVRBS3-inducible gene; a consensus binding sequence from a AVRBS3-inducible gene or synthetic binding sequence engineered therefrom (e.g., UPA DNA-binding domain); TAL; LexA (see, e.g., Brent & Ptashne (1985), supra); LacR (see, e.g., Labow et al. (1990) Mol. Cell. Biol. 10:3343-56; Baim et al. (1991) Proc. Natl. Acad. Sci. USA 88(12):5072-6); a steroid hormone receptor (Elliston et al. (1990) J. Biol. Chem. 265:11517-121); the Tet repressor (U.S. Pat. No. 6,271,341) and a Tet repressor variant that binds to a tet operator sequence in the presence, but not the absence, of tetracycline (Tc); the DNA-binding domain of NF-kappaB; and components of the regulatory system described in Wang et al. (1994) Proc. Natl. Acad. Sci. USA 91(17):8180-4, which utilizes a fusion of GAL4, a hormone receptor, and VP16.

The DNA-binding domain of one or more of the nucleases used in the methods and compositions described herein can comprise a naturally occurring or engineered (non-naturally occurring) TAL effector DNA binding domain. See, e.g., U.S. Patent Publication No. 2011/0301073.

Alternatively, the nuclease can comprise a CRISPR system. For example, the nuclease can comprise a CRISPR/Cas system.

The CRISPR-associated system evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas protein (e.g., a Cas9 protein) to the sequence in the viral genome. The Cas protein cleaves and thereby silences the viral target. Recently, the CRISPR/Cas system has been adapted for genome editing in eukaryotic cells. The introduction of site-specific double strand breaks (DSBs) enables target sequence alteration through one of two endogenous DNA repair mechanisms—either non-homologous end-joining (NHEJ) or homology-directed repair (HDR). The CRISPR/Cas system has also been used for gene regulation including transcription repression and activation without altering the target sequence. Targeted gene regulation based on the CRISPR/Cas system can, for example, use an enzymatically inactive Cas9 (also known as a catalytically dead Cas9).

CRISPR/Cas systems include a CRISPR (clustered regularly interspaced short palindromic repeats) locus, which encodes RNA components of the system, and a Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60). CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in nature in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

For use of the CRISPR/Cas system to create targeted insertions and deletions, the two non-coding RNAs (crRNA and the TracrRNA) can be replaced by a single RNA referred to as a guide RNA (gRNA). Activity of the CRISPR/Cas system comprises three steps: (i) insertion of exogenous DNA sequences into the CRISPR array to prevent future attacks, in a process called “adaptation,” (ii) expression of the relevant proteins, as well as expression and processing of the array, followed by (iii) RNA-mediated interference with the foreign nucleic acid. In the bacterial cell, several Cas proteins are involved with the natural function of the CRISPR/Cas system and serve roles in functions such as insertion of the foreign DNA etc.

The Cas protein can be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Where an animal or cell as described herein is genetically edited using a CRISPR system, a CRISPR/Cas9 system can be used to generate the animal or cell. To use Cas9 to edit genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA can be delivered directly to a cell or target specific gRNA(s) can be to a cell (these RNAs can alternatively be produced by a gene constructed to express these RNAs). Selection of target sites and designed of crRNA/gRNA are well known in the art. A discussion of construction and cloning of gRNAs can be found at http://www(dot)genome-engineering(dot)org/crispr/wp-content/uploads/2014/05/CRISPR-Reagent-Description-Rev20140509.pdf.

A guide RNA of a CRISPR/Cas system or DNA-binding polypeptide can specifically recognize and bind to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Targeting Endonucleases

A DNA-binding polypeptide that specifically recognizes and binds to a target nucleotide sequence can be comprised within a chimeric polypeptide, so as to confer specific binding to the target sequence upon the chimeric polypeptide. In examples, such a chimeric polypeptide may comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide may also comprise other functional polypeptide motifs and/or domains, such as for example and without limitation: a spacer sequence positioned between the functional polypeptides in the chimeric protein; a leader peptide; a peptide that targets the fusion protein to an organelle (e.g., the nucleus); polypeptides that are cleaved by a cellular enzyme; peptide tags (e.g., Myc, His, etc.); and other amino acid sequences that do not interfere with the function of the chimeric polypeptide.

Functional polypeptides (e.g., DNA-binding polypeptides and nuclease polypeptides) in a chimeric polypeptide may be operatively linked. Functional polypeptides of a chimeric polypeptide can be operatively linked by their expression from a single polynucleotide encoding at least the functional polypeptides ligated to each other in-frame, so as to create a chimeric gene encoding a chimeric protein. Alternatively, the functional polypeptides of a chimeric polypeptide can be operatively linked by other means, such as by cross-linkage of independently expressed polypeptides.

A DNA-binding polypeptide, or guide RNA that specifically recognizes and binds to a target nucleotide sequence can be comprised within a natural isolated protein (or variant thereof), wherein the natural isolated protein or variant thereof also comprises a nuclease polypeptide (and may also comprise a recombinase and/or ligase polypeptide). Examples of such isolated proteins include TALENs, recombinases (e.g., Cre, Hin, Tre, and FLP recombinase), RNA-guided CRISPR/Cas9, and meganucleases.

As used herein, the term “targeting endonuclease” refers to natural or engineered isolated proteins and variants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within an ANP32 locus (e.g., either because the target sequence is comprised within the native sequence at the locus, or because the target sequence has been introduced into the locus, for example, by recombination) can be used.

Some examples of suitable chimeric polypeptides include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), RNA-guided CRISPR/Cas9, a meganuclease; and others known to those in the art. Particular examples include a chimeric protein comprising a site-specific DNA binding polypeptide and a nuclease polypeptide. Chimeric polypeptides may be engineered by methods known to those of skill in the art to alter the recognition sequence of a DNA-binding polypeptide comprised within the chimeric polypeptide, so as to target the chimeric polypeptide to a particular nucleotide sequence of interest.

The chimeric polypeptide can comprise a DNA-binding domain (e.g., zinc finger, TAL-effector domain, etc.) and a nuclease (cleavage) domain. The cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Illustrative endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNAse I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, the near edges of the target sites can be separated by 5-8 nucleotides or by 15-18 nucleotides. However, any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/transgenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, fusion proteins can comprise the cleavage domain (or cleavage half-domain) from at least one Type IIS restriction enzyme and one or more zinc finger binding domains, which may or may not be engineered.

An illustrative Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Illustrative Type IIS restriction enzymes are described in U.S. Patent Publication No. 2007/0134796. Additional restriction enzymes also contain separable binding and cleavage domains, and these are contemplated by the present disclosure. See, for example, Roberts et al. (2003) Nucleic Acids Res. 31:418-420.

The cleavage domain can comprise one or more engineered cleavage half-domain (also referred to as dimerization domain variants) that minimize or prevent homodimerization, as described, for example, in U.S. Patent Publication Nos. 2005/0064474; 2006/0188987 and 2008/0131962.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

### Zinc Finger Nucleases

A chimeric polypeptide can comprise a custom-designed zinc finger nuclease (ZFN) that may be designed to deliver a targeted site-specific double-strand DNA break into which an exogenous nucleic acid, or donor DNA, may be integrated (see US Patent publication 2010/0257638). ZFNs are chimeric polypeptides containing a non-specific cleavage domain from a restriction endonuclease (for example, FokI) and a zinc finger DNA-binding domain polypeptide. See, e.g., Huang et al. (1996) J. Protein Chem. 15:481-9; Kim et al. (1997a) Proc. Natl. Acad. Sci. USA 94:3616-20; Kim et al. (1996) Proc. Natl. Acad. Sci. USA 93:1156-60; Kim et al. (1994) Proc. Natl. Acad. Sci. USA 91:883-7; Kim et al. (1997b) Proc. Natl. Acad. Sci. USA 94:12875-9; Kim et al. (1997c) Gene 203:43-9; Kim et al. (1998) Biol. Chem. 379:489-95; Nahon and Raveh (1998) Nucleic Acids Res. 26:1233-9; Smith et al. (1999) Nucleic Acids Res. 27:674-81. The ZFNs can comprise non-canonical zinc finger DNA binding domains (see US Patent publication 2008/0182332). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA. Mani et al. (2005) Biochem. Biophys. Res. Commun. 334:1191-7; Smith et al. (2000) Nucleic Acids Res. 28:3361-9. Dimerization of the ZFN can be facilitated by two adjacent, oppositely oriented DNA-binding sites. Id.

A method for the site-specific integration of an exogenous nucleic acid into at least one ANP32 locus of a host can comprise introducing into a cell of the host a ZFN, wherein the ZFN recognizes and binds to a target nucleotide sequence, wherein the target nucleotide sequence is comprised within at least one ANP32 locus of the host. In certain examples, the target nucleotide sequence is not comprised within the genome of the host at any other position than the at least one ANP32 locus. For example, a DNA-binding polypeptide of the ZFN may be engineered to recognize and bind to a target nucleotide sequence identified within the at least one ANP32 locus (e.g., by sequencing the ANP32 locus). A method for the site-specific integration of an exogenous nucleic acid into at least one ANP32 performance locus of a host that comprises introducing into a cell of the host a ZFN may also comprise introducing into the cell an exogenous nucleic acid, wherein recombination of the exogenous nucleic acid into a nucleic acid of the host comprising the at least one ANP32 locus is facilitated by site-specific recognition and binding of the ZFN to the target sequence (and subsequent cleavage of the nucleic acid comprising the ANP32 locus).

### Optional Exogenous Nucleic Acids for Integration at an ANP32 Locus

Exogenous nucleic acids for integration at an ANP32 locus include: an exogenous nucleic acid for site-specific integration in at least one ANP32 locus, for example and without limitation, an ORF; a nucleic acid comprising a nucleotide sequence encoding a targeting endonuclease; and a vector comprising at least one of either or both of the foregoing. Thus, particular nucleic acids include nucleotide sequences encoding a polypeptide, structural nucleotide sequences, and/or DNA-binding polypeptide recognition and binding sites.

### Optional Exogenous Nucleic Acid Molecules for Site-Specific Integration

As noted above, insertion of an exogenous sequence (also called a “donor sequence” or “donor” or “transgene”) is provided, for example for expression of a polypeptide, correction of a mutant gene, or for increased expression of a wild-type gene. It will be readily apparent that the donor sequence is typically not identical to the genomic sequence where it is placed. A donor sequence can contain a non-homologous sequence flanked by two regions of homology to allow for efficient homology-directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. See e.g., U.S. Patent Publication Nos. 2010/0047805, 2011/0281361, 2011/0207221, and 2013/0326645. If introduced in linear form, the ends of the donor sequence can be protected (e.g. from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLY)).

The donor is generally integrated so that its expression is driven by the endogenous promoter at the integration site, namely the promoter that drives expression of the endogenous gene into which the donor is integrated (e.g., ANP32). However, it will be apparent that the donor may comprise a promoter and/or enhancer, for example a constitutive promoter or an inducible or tissue specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Exogenous nucleic acids that may be integrated in a site-specific manner into at least one ANP32 locus, so as to modify the ANP32 locus include, for example and without limitation, nucleic acids comprising a nucleotide sequence encoding a polypeptide of interest; nucleic acids comprising an agronomic gene; nucleic acids comprising a nucleotide sequence encoding an RNAi molecule; or nucleic acids that disrupt the ANP32 gene.

An exogenous nucleic acid can be integrated at a ANP32 locus, so as to modify the ANP32 locus, wherein the nucleic acid comprises a nucleotide sequence encoding a polypeptide of interest, such that the nucleotide sequence is expressed in the host from the ANP32 locus. In some examples, the polypeptide of interest (e.g., a foreign protein) is expressed from a nucleotide sequence encoding the polypeptide of interest in commercial quantities. In such examples, the polypeptide of interest may be extracted from the host cell, tissue, or biomass.

### Nucleic Acid Molecules Comprising a Nucleotide Sequence Encoding a Targeting Endonuclease

A nucleotide sequence encoding a targeting endonuclease can be engineered by manipulation (e.g., ligation) of native nucleotide sequences encoding polypeptides comprised within the targeting endonuclease. For example, the nucleotide sequence of a gene encoding a protein comprising a DNA-binding polypeptide may be inspected to identify the nucleotide sequence of the gene that corresponds to the DNA-binding polypeptide, and that nucleotide sequence may be used as an element of a nucleotide sequence encoding a targeting endonuclease comprising the DNA-binding polypeptide. Alternatively, the amino acid sequence of a targeting endonuclease may be used to deduce a nucleotide sequence encoding the targeting endonuclease, for example, according to the degeneracy of the genetic code.

In illustrative nucleic acid molecules comprising a nucleotide sequence encoding a targeting endonuclease, the last codon of a first polynucleotide sequence encoding a nuclease polypeptide, and the first codon of a second polynucleotide sequence encoding a DNA-binding polypeptide, may be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. The last codon (i.e., most 3′ in the nucleic acid sequence) of a first polynucleotide sequence encoding a nuclease polypeptide, and a second polynucleotide sequence encoding a DNA-binding polypeptide, can be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto, or separated therefrom by no more than a short peptide sequence.

A sequence separating polynucleotide sequences encoding functional polypeptides in a targeting endonuclease (e.g., a DNA-binding polypeptide and a nuclease polypeptide) may, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not interfere with the respective functions of these structures.

### Other Knockout Methods

Various other techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques. An animal that is genomically edited is an animal wherein all of its cells have the edit, including its germ line cells. When methods are used that produce an animal that is mosaic in its modification, the animals may be inbred and progeny that are genomically edited may be selected. Cloning, for instance, may be used to make a mosaic animal if its cells are modified at the blastocyst state, or genomic editing can take place when a single cell is edited. Animals that are edited so they do not sexually mature can be homozygous or heterozygous for the edit, depending on the specific approach that is used. If a particular gene is inactivated by a knockout edit, homozygosity would normally be required. If a particular gene is inactivated by an RNA interference or dominant negative strategy, then heterozygosity is often adequate.

Typically, in embryo/zygote microinjection, a nucleic acid construct or mRNA is introduced into a fertilized egg; one or two cell fertilized eggs are used as the nuclear structure containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 400,000 sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on the boar. All fertilizing oocytes can be incubated at 38.7° C. in 5.0% CO₂ atmosphere for six hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the gene edited animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch catheter. After surgery, real-time ultrasound examination of pregnancy can be performed.

In somatic cell nuclear transfer, a transgenic or gene edited cell such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic or gene edited cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al. (1998) Science 280, 1256-1258 and U.S. Pat. Nos. 6,548,741, 7,547,816, 7,989,657, or 6,211,429. For pigs, recipient females can be checked for pregnancy approximately 20-21 days after transfer of the embryos.

Standard breeding techniques can be used to create animals that are homozygous for the inactivated gene from the initial heterozygous founder animals. Homozygosity may not be required, however. Gene edited pigs described herein can be bred with other pigs of interest.

Once gene edited animals have been generated, inactivation of an endogenous nucleic acid can be assessed using standard techniques. Initial screening can be accomplished by Southern blot analysis to determine whether or not inactivation has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12, 1; Guatelli et al. (1990) Proc. Natl. Acad. Sci. USA 87:1874; and Weiss (1991) Science 254:1292. At the blastocyst stage, embryos can be individually processed for analysis by PCR, Southern hybridization and splinkerette PCR (see, e.g., Dupuy et al. Proc. Nat'l Acad. Sci. USA (2002) 99:4495).

### Interfering RNAs

A variety of interfering RNA (RNAi) systems are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNAse, e.g., Dicer) and ssRNAse (e.g., Argonaut 2 or Ago2). RISC utilizes antisense strand as a guide to find a cleavable target. Both siRNAs and microRNAs (miRNAs) are known. A method of inactivating a gene in a genetically edited animal comprises inducing RNA interference against a target gene and/or nucleic acid such that expression of the target gene and/or nucleic acid is reduced.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

In vitro cells, in vivo cells, or a genetically edited animal such as an ungulate animal that express an RNAi directed against a gene encoding an ANP32 protein can be used. The RNAi may be, for instance, selected from the group consisting of siRNA, shRNA, dsRNA, RISC and miRNA.

### Inducible Systems

An inducible system may be used to inactivate an ANP32 gene. Various inducible systems are known that allow spatial and temporal control of inactivation of a gene. Several have been proven to be functional in vivo in porcine animals.

An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another line of animals expresses the acceptor, in which the expression of the gene of interest (or the gene to be altered) is under the control of the target sequence for the tTA/rtTA transactivators (or is flanked by loxP sequences). Mating the two of animals provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic and/or gene edited animal, using either spatial control (with a tissue- or cell-specific promoter), or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically edited animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

In vitro cells, in vivo cells, or a genetically edited animal such as an ungulate animal that comprises an ANP32 gene under control of an inducible system can be used. The chromosomal modification of an animal may be genomic or mosaic. The inducible system may be, for instance, selected from the group consisting of Tet-On, Tet-Off, Cre-lox, and Hif1 alpha.

### Vectors and Nucleic Acids

A variety of nucleic acids may be introduced into cells for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. A promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. For example, a fusion of the chicken beta actin gene promoter and the CMV enhancer can be used as a promoter. See, for example, Xu et al. (2001) Hum. Gene Ther. 12:563; and Kiwaki et al. (1996) Hum. Gene Ther. 7:821.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

A sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain animals with conditional expression of a transgene. For example, a promoter driving expression of the marker/transgene can be either ubiquitous or tissue-specific, which would result in the ubiquitous or tissue-specific expression of the marker in FO animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

The exogenous nucleic acid can encode a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG tag (Kodak, New Haven, Conn.).

Nucleic acid constructs can be methylated using a SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult animal cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl.1):S7; Minos (Pavlopoulos et al. (2007) Genome Biology 8(Suppl.1):S2); Hsmar1 (Miskey et al. (2007)) Mol. Cell Biol. 27:4589); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

### Founder Animals, Animal Lines, Traits, and Reproduction

Founder animals may be produced by cloning and other methods described herein. The founders can be homozygous for a gene edit, as in the case where a zygote or a primary cell undergoes a homozygous modification. Similarly, founders can also be made that are heterozygous. In the case of the animals comprising at least one modified chromosomal sequence in a gene encoding an ANP32 protein, the founders are preferably heterozygous. The founders may be genomically edited, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically edited. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40 (1994) 123-137, U.S. Pat. No. 7,709,206, US 2001/0016315, US 2011/0023140, and US 2005/0153317. An animal line may include a trait chosen from a trait in the group consisting of a production trait, a type trait, a workability trait, a fertility trait, a mothering trait, and a disease resistance trait. Further traits include expression of a recombinant gene product.

In addition to monitoring traits, artisans can look at the genetic background of the animal as the whole. Genomic edits such as those of the present teachings may be made in elite genetic backgrounds. Elite PIC™ (Pig Improvement Company, Limited, Basingstoke, UK) lines 2, 3, 15, 19, 27, 62 and 65 are lines selected for superior commercial phenotypes. Fibroblast cell lines were grown from collagenase treated ear notch samples extracted from the animals from these cell lines deposited with the American Type Culture Collection (ATCC®). The ATCC® has an address of 10801 University Boulevard, Manassas, VA 20110-2209. A representative sample of PIC™ Line 2 was deposited with the ATCC on Apr. 3, 2019 and assigned ATTC® Patent Deposit Number PTA-125814. A representative sample of PIC™ Line 3 was deposited with the ATCC on Apr. 3, 2019 and assigned ATTC® Patent Deposit Number PTA-125815. A representative sample of PIC™ Line 15 was deposited with the ATCC on Apr. 3, 2019 and assigned ATTC® Patent Deposit Number PTA-125816. A representative sample of PIC™ Line 65 was deposited with the ATCC® on Apr. 3, 2019 and assigned ATTC® Patent Deposit Number PTA-125813. A representative sample of PIC™ Line 19 was deposited with the ATCC ° on Apr. 3, 2019 and assigned ATTC® Patent Deposit Number PTA-125811. A representative sample of PIC™ Line 27 was deposited with the ATCC on Apr. 3, 2019 and assigned ATTC® Patent Deposit Number PTA-125907. A representative sample of PIC™ Line 62 was deposited with the ATCC on Apr. 3, 2019 and assigned ATTC® Patent Deposit Number PTA-125812. Each deposit was made according to the Budapest Treaty. Upon issuance, all restrictions on the availability to the public of the deposit will be irrevocably removed consistent with all of the requirements of the Budapest Treaty and 37 C.F.R. §§ 1.801-1.809. Applicant does not waive any infringement of rights granted under this patent.

Other potential porcine lines include lines can be PIC™ Line 15, PIC™ Line 17, PIC™ Line 27, PIC™ Line 65, PIC™ Line 14, PIC™ Line 62, PIC337, PIC800, PIC280, PIC327, PIC408, PIC™ 399, PIC410, PIC415, PIC359, PIC380, PIC837, PIC260, PIC265, PIC210, PIC™ Line 2, PIC™ Line 3, PIC™ Line 4, PIC™ Line 5, PIC™ Line 18, PIC™ Line 19, PIC™ Line 92, PIC95, PIC™ CAMBOROUGH® (Pig Improvement Company, Limited, Basingstoke, UK), PIC1070, PIC™ CAMBOROUGH® 40, PIC™ CAMBOROUGH® 22, PIC1050, PIC™ CAMBOROUGH® 29, PIC™ CAMBOROUGH® 48, or PIC™ CAMBOROUGH® x54.

In various aspects, PIC™ Line 65 is sold under the trade name PIC337. In various aspects, PIC™ line 62 is sold under the tradename PIC408. In various aspects, hybrid pigs made by crossing PIC™ lines 15 and 17 are sold under the tradenames PIC800 or PIC280. In various aspects, PIC™ Line 27 is sold under the tradename PIC327. In various aspects, hybrids created from crossing PIC™ Line 65 and PIC™ Line 62 are sold under the tradenames PIC399, PIC410, or PIC415. In various aspects, hybrids created from crossing PIC™ Line 65 and Pic Line 27 are sold under the tradename PIC359. In various aspects, hybrids prepared from crossing PIC™ Line 800 pigs (which is a hybrid of PIC™ Line 15 and PIC™ Line 17) to PIC™ Line 65 pigs are sold under the tradenames PIC380 or PIC837. In various aspects, PIC™ Line 14 is sold under the trade name PIC260. In various aspects, hybrids created from crossing PIC™ Line 14 and PIC™ Line 65 are sold under the tradename PIC265. In various aspects, hybrids created by crossing PIC™ Line 2 and PIC™ Line 3 are sold under the tradenames PIC210, PIC™ CAMBOROUGH®, and PIC1050. In various aspects, hybrids of PIC™ Line 3 and PIC™ Line 92 are sold under the tradename PIC95. In various configurations, hybrids made from crossing PIC™ Line 19 and PIC™ Line 3 are sold under the tradename PIC1070. In various aspects, hybrids created by crossing PIC™ Line 18 and PIC™ Line 3 are sold under the tradename PIC™ CAMBOROUGH® 40. In various aspects, hybrids created from crossing PIC™ Line 19 and PIC1050 (which is itself a hybrid of PIC™ lines 2 and 3) are sold under the tradename PIC™ CAMBOROUGH® 22. In various aspects, hybrids created from crossing PIC™ Line 2 and PIC1070 (which is itself a hybrid of PIC™ lines 19 and 3) are sold under the tradename PIC™ CAMBOROUGH® 29. In various aspects, hybrids created from crossing PIC™ Line 18 and PIC1050 (which is itself a hybrid of PIC™ lines 2 and 3) are sold under the tradename PIC™ CAMBOROUGH® 48. In various aspects, hybrids created from crossing PIC™ Line 4 and PIC™ Line 5 are sold under the tradename PIC™ CAMBOROUGH® x54.

Animals with a desired trait or traits may be modified to prevent their sexual maturation. Since the animals are sterile until matured, it is possible to regulate sexual maturity as a means of controlling dissemination of the animals. Animals that have been bred or modified to have one or more traits can thus be provided to recipients with a reduced risk that the recipients will breed the animals and appropriate the value of the traits to themselves. For example, the genome of an animal can be modified, wherein the modification comprises inactivation of a sexual maturation gene, wherein the sexual maturation gene in a wild type animal expresses a factor selective for sexual maturation. The animal can be treated by administering a compound to remedy a deficiency caused by the loss of expression of the gene to induce sexual maturation in the animal.

Breeding of animals that require administration of a compound to induce sexual maturity may advantageously be accomplished at a treatment facility. The treatment facility can implement standardized protocols on well-controlled stock to efficiently produce consistent animals. The animal progeny may be distributed to a plurality of locations to be raised. Farms and farmers (a term including a ranch and ranchers) may thus order a desired number of progeny with a specified range of ages and/or weights and/or traits and have them delivered at a desired time and/or location. The recipients, e.g., farmers, may then raise the animals and deliver them to market as they desire.

A genetically edited ungulate animal having an inactivated sexual maturation gene can be delivered (e.g., to one or more locations, to a plurality of farms). The animals can have an age of between about 1 day and about 180 days. The animal can have one or more traits (for example one that expresses a desired trait or a high-value trait or a novel trait or a recombinant trait).

### Detection of Edited Pigs

One useful method of detecting the desired edit is to use real-time PCR. PCR primers flanking the region of interest and a probe that specifically anneals to the region of interest are designed. The probe is labelled with both a fluorophore and a quencher. The probe can have 50%, 60%, 70%, 80%, 90%, 95%, or 100% homology with the desired sequence. In the PCR reaction, the primers and probe hybridize in a sequence-dependent manner to the complementary DNA strand of the region of interest. Because the probe is intact, the fluorophore and quencher are in close proximity and the quencher absorbs fluorescence emitted by the fluorophore. The polymerase extends from the primers and begins DNA synthesis. When the polymerase reaches the probe, the exonuclease activity of the polymerase cleaves the hybridized probe. As a result of cleavage, the fluorophore is separated from the quencher and fluoresces. This fluorescence is detected by the real time instrument. These steps are repeated for each PCR cycle and allow detection of specific products.

For example, three separate sets of primers and probes can be designed for two assays. Each assay can have two sets of primers. The first set of primers can flank the unedited genomic sequence of a gRNA used in an edit (SEQ ID NOs: 26 or 19 for ANP32A or SEQ ID NOs: 55 and 59 for ANP32B) and a probe which binds to the unedited genomic DNA in between the primers. The probe can be 50%, 60%, 70%, 80%, 90%, 95%, or 100% homologous to the unedited genomic sequence between the primers. There can be a separate set for each guide RNA used in the edit. The final sets of primers can flank the desired exogenous stop codon created by excision of the sequence between the cut sites of the guides. A probe can be designed to bind the desired edit in between these primers with 50%, 60%, 70%, 80%, 90%, 95%, or 100% homology to the desired edit. A commercial real-time PCR kit can then be used to probe various animals for the desired edit. A variety of commercial real-time PCR kits exist including, such as, but without limitation, PRIMETIME® from IDT, TAQMAN® (Roche Molecular Systems, Inc, Pleasonton, CA) from Applied Biosystems, and various kits from Qiagen and Bio-Rad. Skilled persons will recognize that any such kit can be used with the primers and methods of the present teachings to achieve like results.

Having described the invention in detail, it will be apparent that modifications and variations are possible without departing from the scope of the invention defined in the appended claims.

### EXAMPLES

The following non-limiting examples are provided to further illustrate the present invention.

### Example 1. Generation of Pigs Having Edited ANP32 Genes

To generate pigs having edited ANP32 genes, editing strategies were designed to introduce stop codons into the porcine ANP32A and ANP32B genes in conserved exonic sequences upstream of the coding region that codes for N129 and D130. The ANP32A and ANP32B guide RNAs listed above in Tables 6 and 7 will be assessed for on- and off-target activity in porcine fibroblasts.

ANP32A has two transcripts: ENSSSCT00000070641.1 and ENSSSCT00000005475.4. Guides in the conserved region of exon 2 (chr1: 166,671,648-166,671,797) and exon 3 (chr1: 166,671,387-166,671,509) will be tested for on-target activity.

ANP32B (NANS) has seven transcripts: ENSSSCT00000062686.2, ENSSSCT00000055524.2, ENSSSCT00000068404.1, ENSSSCT00000045745.2, ENSSSCT00000005912.4, ENSSSCT00000054395.2, and ENSSSCT00000044227.2. Guides in exon 2 (chr1:239,852,885-239,853,034) and exon 3 (chr1: 239,856,334-239,856,456) will be tested for on-target activity.

These data will be then used to select CRISPR reagents to generate knockout animals by zygote microinjection and embryo transfer.

Pigs having a knockout of ANP32A alone, ANP32B alone, or both ANP32A and ANP32B will be generated. These animals will be assessed to confirm that they have the desired gene edit and will then be evaluated for resistance to IAV infection. Resistance to IAV infection will be assessed in vitro using fibroblast cells obtained from the animals and in live animals. The cells and animals will be exposed to a variety of IAVs in order to assess resistance.

### Example 2. Use of gRNA Pairs to Introduce Premature Stop Codons into the Coding Regions of ANP32A and ANP32B

This example demonstrates the use of gRNA pairs to introduce premature stop codons into the coding regions of ANP32A and ANP32B. The introduction of premature stop codons in ANP32A and ANP32B will create truncated and non-functional proteins, or could initiate nonsense-mediated mRNA decay resulting in elimination of ANP32A and ANP32B mRNA transcripts. Stop codons can be introduced via the homology directed repair (HDR) pathway by inclusion of a single-stranded DNA template in editing experiments. However, single-stranded DNA can integrate randomly in the genome. Therefore, it would be advantageous to identify gRNA pairs that result in-frame stop codons, without the introduction of non-wildtype amino acids. To accomplish this, two gRNAs can be used to direct nuclease cut sites, which are then repaired by NHEJ in an end-to-end manner.

Guides within ANP32A and ANP32B were tested computationally for their ability to generate in-frame stop codons when paired. Computational predictions were subsequently tested in porcine fetal fibroblasts, as described below. The gRNAs listed in Table 8 below were generated by in vitro transcription and complexed with SpyCas9 in water, using 3.2 μg of Cas9 protein and 2.2 μg of gRNA in a total volume of 2.23 μl. Half volumes (1.115 μl) of the resulting ribonucleoprotein (RNP) complexes were then combined 1:1 in a total volume of 2.23 μl to generate pairs, as indicated in Table 8, and nucleofected into porcine fetal fibroblasts (PFFs) using a Lonza electroporator. In preparation for nucleofection, PFF cells were harvested using TRYPLE EXPRESS™ (ThermoFisher Scientific, Waltham, MA, recombinant Trypsin). Specifically, the culture medium was removed from cells, cells were washed once with Hank's Balanced Salt Solution (HBSS) or Dulbecco's Phosphate-Buffered Saline (DPBS), and incubated for 3-5 minutes at 38.5° C. in the presence of TrypLE. Cells were then harvested with complete medium. Cells were pelleted via centrifugation (300 g×5 minutes at room temperature), supernatant was discarded, and then the cells were resuspended in 10 mL PBS to obtain single cell suspensions to allow cell counting using trypan blue staining. After counting, cells were pelleted via centrifugation, the supernatant was discarded, and the cells were resuspended in nucleofection buffer P3 at a final concentration of 7.5×10⁶ cells/ml. 20 μl of the cell suspension was added to each well of a nucleofection cuvette containing the RNP mixture and then mixed gently to resuspend the cells. The RNP/cell mixture was then nucleofected with program CM138. Following nucleofection, 80 μl of warm Embryonic Fibroblast Medium (EFM) [Dulbecco's Modified Eagle's Medium (DMEM) containing 2.77 mM glucose, 1.99 mM L-glutamine, and 0.5 mM sodium pyruvate, supplemented with 100 μM 2-Mercaptoethanol, 1× Eagle's minimum essential medium non-essential amino acids (MEM NEAA), 100 μg/mL Penicillin-Streptomycin, and 12% Fetal Bovine Serum] was added to each well. The suspensions were mixed gently by pipetting, and then 100 μl were transferred to a 12-well plate containing 900 μl of EFM pre-incubated at 38.5° C. The plate was then incubated at 38.5° C., 5% CO₂ for 48 hours. Forty-eight hours after nucleofection, genomic DNA was prepared from transfected and control PFF cells. Specifically, 15 μl of QUICKEXTRACT™ DNA Extraction Solution (Lucigen Corporation, Middleton, WI) were added to pelleted cells, and the cells were then lysed by incubating for 10 minutes at 37° C., for 8 minutes at 65° C., and for 5 minutes at 95° C. Lysate was held at 4° C. until used for DNA sequencing.

To evaluate NHEJ repair outcomes at ANP32A and ANP32B target sites mediated by the guide RNA/Cas endonuclease system, a region of approximately 250 bp of genomic DNA surrounding the target site was amplified by PCR and the PCR product was then examined by amplicon deep sequencing for the presence and nature of repairs. After transfection in triplicate, PFF genomic DNA was extracted and the region surrounding the intended target site was PCR amplified with NEB Q5 Polymerase adding sequences necessary for amplicon-specific barcodes and Illumina sequencing using “tailed” primers through two rounds of PCR, respectively. The resulting PCR amplifications were deep sequenced on an Illumina MiSeq Personal Sequencer. The resulting reads were examined for the presence and nature of repairs at the expected sites of cleavage by comparison to control experiments where the Cas9 protein and guide RNA were omitted from the transfection or by comparison to the reference genome. To calculate the frequency of NHEJ mutations for a target site/Cas9 protein/guide RNA combination, the total number of mutant reads (amplicon sequences containing insertions or deletions when compared to the DNA sequences from control treatments or reference genome) was divided by total read number (wild-type plus mutant reads) of an appropriate length containing a perfect match to the barcode and forward primer. Total read counts averaged approximately 7000 per sample and NHEJ activity is expressed as the average (n=3) mutant fraction in Table 8.

A subset of gRNA pairs screened in porcine fetal fibroblasts were additionally tested for their ability to introduce a premature stop codon in the ANP32A and ANP32B coding regions in porcine embryos. The subset of guides to be tested in porcine embryos was chosen based on their efficacy in generating premature stop codons in porcine fibroblasts. Edited porcine embryos were generated as described below. Briefly, oocytes recovered from slaughterhouse ovaries were in vitro fertilized. The sgRNP solution was injected into the cytoplasm of presumptive zygotes at 16-17 hours post-fertilization by using a single pulse from a FemtoJet 4i microinjector (Eppendorf; Hamburg, Germany) with settings at pi=200 hPa, ti=0.25 s, pc=15 hPa. Glass capillary pipettes with an outer diameter of 1.2 mm and an inner diameter of 0.94 mm were pulled to a very fine point of <0.5 μm (Sutter Instrument, Navato, CA, USA). Microinjection was performed in TL-Hepes (ABT360, LLC) supplemented with 3 mg/ml BSA (Proliant) on the heated stage of an inverted microscope equipped with Narishige (Narishige International USA, Amityville, NY) micromanipulators. Following injections, presumptive zygotes were cultured for 7 days in BO-IVC (IVF Bioscience, Falmouth, Cornwall, UK) in an incubator environment of 5% CO₂, 5% O₂, 90% N₂. Mutation frequency of blastocysts was determined by Illumina sequencing as described for fetal fibroblasts above. The frequencies of end-to-end NHEJ repairs resulting in premature stop codons in ANP32A and ANP32B are shown in Table 8.

This example demonstrates that the porcine ANP32A and ANP32B gene nucleotide sequences can be edited through the stimulation of double-strand breaks mediated by transfecting Cas9 protein with paired guide RNAs to create a de novo in-frame stop codon.

TABLE 8
Use of gRNA pairs to introduce premature stop codons
into the coding regions of ANP32A and ANP32B

| | | | Freq. desired | Freq. desired edit |
|---|---|---|---|---|
| Gene | Guide 1 | Guide 2 | edit PFFs (%) | embryos(%) |
| ANP32A | SEQ ID NO: 33 | SEQ ID NO: 36 | 0 | Not tested |
| ANP32A | SEQ ID NO: 33 | SEQ ID NO: 40 | 63.7 | 6.2 |
| ANP32A | SEQ ID NO: 15 | SEQ ID NO: 22 | 42.7 | Not tested |
| ANP32A | SEQ ID NO: 15 | SEQ ID NO: 23 | 22.3 | Not tested |
| ANP32A | SEQ ID NO: 16 | SEQ ID NO: 24 | 11.7 | Not tested |
| ANP32A | SEQ ID NO: 17 | SEQ ID NO: 24 | 14.3 | Not tested |
| ANP32A | SEQ ID NO: 19 | SEQ ID NO: 26 | 54 | 48.5 |
| ANP32B | SEQ ID NO: 44 | SEQ ID NO: 46 | 17 | 8.0 |
| ANP32B | SEQ ID NO: 55 | SEQ ID NO: 58 | 65 | 29.3 |
| ANP32B | SEQ ID NO: 55 | SEQ ID NO: 59 | 71.7 | 36.8 |

### Example 3 Editing of Pigs to Introduce Premature Stop Codons

Porcine oocytes were isolated, fertilized, and then the resulting zygotes are edited to generate gene edited pigs.

RNP complexes were microinjected into the cytoplasm of in vivo or in vitro fertilized porcine one-cell zygotes. These zygotes were then incubated to generate edited multicellular embryos and transferred to surrogate gilts via standard methods to birth gene edited pigs. To prepare embryo donors and surrogates, pubertal gilts from PIC™ Line 2, Line 3, Line 15, and Line 65 were subjected to estrus synchronization by treatment with altrenogest solution (20-36 mg/animal) for 14 days. Follicular growth was induced by the administration of PMSG 36 hours following the last dose of Matrix, and ovulation was induced by the administration of hCG 82 hours after PMSG administration. To generate in vivo fertilized zygotes, females in standing heat were then artificially inseminated (AI) with boar semen from the corresponding PIC™ line. In vivo derived zygotes were recovered surgically 12-24 hours after AI by retrograde flushing the oviduct with sterile TL-HEPES medium supplemented with 0.3% BSA (w/v). Fertilized zygotes were subjected to a single 2-50 picoliter (pl) cytoplasmic injection of Cas9 protein and guide RNA complex (25-50 ng/μl and 12.5-35 ng/μl) targeting ANP32 and cultured in PZM5 medium (Yoshioka, K., et al., Biol. Reprod., 2002, 60: 112-119; Suzuki, C., et al., Reprod. Fertil. Dev., 2006 18, 789-795; Yoshioka, K., J. Reprod. Dev. 2008, 54, 208-213). Injected zygotes were surgically implanted into the oviducts of estrus synchronized, un-mated surrogate females by a mid-line laparotomy under general anesthesia (each surrogate received 20-60 injected embryos).

In vitro fertilized embryos for gene editing were derived from non-fertilized PIC™ oocytes. Immature oocytes from estrus synchronized PIC™ gilts were collected from medium size (3-6 mm) follicles. Oocytes with evenly dark cytoplasm and intact surrounding cumulus cells were then selected for maturation. Cumulus oocyte complexes were placed in a well containing 500 μl of maturation medium, TCM-199 (Invitrogen) with 3.05 mM glucose, 0.91 mM sodium pyruvate, 0.57 mM cysteine, 10 ng/ml EGF, 0.5 μg/ml luteinizing hormone (LH), 0.5 μg/ml FSH, 10 ng/ml gentamicin (Sigma), and 10% follicular fluid for 42-44 h at 38.5° C. and 5% CO₂, in humidified air. At the end of the maturation, the surrounding cumulus cells were removed from the oocytes by vortexing for 3 min in the presence of 0.1% hyaluronidase. Then, in vitro matured oocytes were placed in 100 μl droplets of IVF medium (modified Tris-buffered medium containing 113.1 mM NaCl, 3 mM KCl, 7.5 mM CaCl₂, 11 mM glucose, 20 mM Tris, 2 mM caffeine, 5 mM sodium pyruvate, and 2 mg/ml bovine serum albumin (BSA)) in groups of 25-30 oocytes and were fertilized according to established protocol (Abeydeera, Biol. Reprod., 57:729-734, 1997) using fresh extended boar semen. One ml of extended semen was mixed with Dulbecco's Phosphate Buffered Saline (DPBS) containing 1 mg/ml BSA to a final volume of 10 ml and centrifuged at 1000×g, 25° C. for 4 minutes, and spermatozoa were washed in DPBS three times. After the final wash, spermatozoa were re-suspended in mTBM medium and added to oocytes at a final concentration of 1×10 5 spermatozoa/ml, and co-incubated for 4-5 h at 38.5° C. and 5% CO₂. Presumptive zygotes were microinjected 5 hours post IVF and transferred to a surrogate female after 18-42 hours (1-4 cell stage). Each surrogate receives injected embryos. Pregnancies were confirmed by lack of return to estrus (21 days) and ultrasound at 28 days post embryo transfer.

To establish the frequency of Cas9-guide RNA targeted gene editing in porcine embryos, uninjected control zygotes and injected surplus zygotes generated by in vitro fertilization were cultivated in PZM3 or PZM5 medium at 38.5° C. for 5-7 days. Blastocysts were harvested at day 7 post cultivation and the genomic DNA isolated for next generation sequencing.

### Example 4 Molecular Characterization of Gene Edited Pigs

This example illustrates the molecular characterization of edited animal genomes.

A tissue sample was taken from an animal whose genome was been edited according to the examples herein. Tail, ear notch, or blood samples were suitable tissue types. The tissue sample was frozen at −20° C. within 1 hour of sampling to preserve integrity of the DNA in the tissue sample.

DNA was extracted from tissue samples after proteinase K digestion in lysis buffer. Characterization was performed on two different sequence platforms, short sequence reads using the ILLUMINA® platform (ILLUMINA®, San Deigo, CA) and long sequence reads on an Oxford NANOPORE™ platform (Oxford NANOPORE™ Technologies, Oxford, UK).

For short sequence reads, two-step PCR was used to amplify and sequence the region of interest. The first step was a locus-specific PCR which amplified the locus of interest from the DNA sample using a combined locus-specific primer with a vendor-specific primer. The second step attached the sequencing index and adaptor sequences to the amplicon from the first step so that sequencing could occur.

The locus-specific primers for the first step PCR were chosen so that they amplified a region <300 bp such that ILLUMINA® paired-end sequencing reads could span the amplified fragment. Multiple amplicons were preferred to provide redundancy should deletions or naturally occurring point mutations prevent primers from correctly binding. Sequence data for the amplicon was generated using an ILLUMINA® sequencing platform (MISEQ®, ILLUMINA®, San Diego, CA). Sequence reads are analyzed to characterize the outcome of the editing process.

For long sequence reads, two-step PCR was used to amplify and sequence the region of interest. The first step is a locus-specific PCR which amplified the locus of interest from the DNA sample using a combined locus-specific primer with a vendor-specific adapter. The second step PCR attached the sequencing index to the amplicon from the first-step PCR so that the DNA was ready for preparing a sequencing library. The step 2 PCR products underwent a set of chemical reactions from a vendor kit to polish the ends of the DNA and ligate on the adapter containing the motor protein to allow access to the pores for DNA strand-based sequencing.

The locus specific primers for the first step PCR range were designed to amplify different regions of the ANP32 gene and amplified regions differed in length. Normalized DNA is then mixed with vendor supplied loading buffer and is loaded onto the NANOPORE™ flowcell.

Long sequence reads, while having lower per base accuracy than short reads, are very useful for observing the long range context of the sequence around the target site.

### Example 5 Challenge of ANP32A Gene Edited Pigs with Influenza Virus

This example illustrates ANP32A edited pigs challenged with influenza virus.

PIC™ pigs were edited using guide RNAs SEQ ID NO: 26 and SEQ ID NO:19 as described in example 3 to create pigs having an edited ANP32A gene comprising SEQ ID NO: 7577. Edits were confirmed as described in Example 4. Edited pigs will be crossbred to create pigs that are homozygous for the edit. These homozygous edited pigs will be inoculated with influenza virus, administered both intramuscularly and intranasally. Serum samples will be obtained on Day 0 (prior to inoculation on that day), Day 3, Day 5, Day 10, Day 14, and Day 21. Realtime PCR will be performed using standard kits to determine the presence of virus in the serum samples according to manufacturer directions.

### Example 6 Challenge of ANP32B Gene Edited Pigs with Influenza Virus

This example illustrates ANP32B edited pigs challenged with influenza virus.

PIC™ pigs were edited using guide RNAs SEQ ID NO: 55 and SEQ ID NO:59 as described in example 3 to create pigs having an edited ANP32B gene comprising SEQ ID NO: 7578. Edits were confirmed as described in Example 4. Edited pigs will be crossbred to create pigs that are homozygous for the edit. These homozygous edited pigs will be inoculated with influenza virus, administered both intramuscularly and intranasally. Serum samples will be obtained on Day 0 (prior to inoculation on that day), Day 3, Day 5, Day 10, Day 14, and Day 21. Realtime PCR will be performed using standard kits to determine the presence of virus in the serum samples according to manufacturer directions.

### Example 7 Assay for Real Time PCR Verification of Desired ANP32A Edit

There will be two assays created for the ANP32 edit detection using real time PCR (rtPCR). For the first assay, two sets of primers and two probes will be designed. One set of primers will flank the spacer sequence set forth in SEQ ID NO: 26. A probe, labeled with a fluorescent moiety, will be designed to anneal to the unedited version of the spacer sequence. The other set of primers will be designed to flank the desired edit sequence (SEQ ID NO: 7577). A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to the y axis (representing the fluorescent moiety for the probe annealing to SEQ ID NO: 7577), the heterozygotes group near the center of the chart, and the wild type pigs group close to the X axis (representing the fluorescent moiety for the probe annealing to SEQ ID NO: 26).

For the second assay, two sets of primers and two probes will also be designed. One set of primers will flank the spacer sequence set forth in SEQ ID NO: 19. A probe, labeled with a fluorescent moiety, will be designed to anneal to the unedited version of the spacer sequence. The other set of primers will be designed to flank the desired edit sequence (SEQ ID NO: 7577)—these may be the same primers and probe used for the first assay. A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to they axis (representing the fluorescent moiety for the probe annealing to SEQ ID NO: 7577), the heterozygotes group near the center of the chart, and the wild type pigs group close to the X axis (representing the fluorescent moiety for the probe annealing to SEQ ID NO: 19).

### Example 8 Assay for Real Time PCR Verification of Desired ANP32B Edit

There will be two assays created for the ANP32B edit detection using real time PCR (rtPCR). For the first assay, two sets of primers and two probes will be designed. One set of primers will flank the spacer sequence set forth in SEQ ID NO: 55. A probe, labeled with a fluorescent moiety, will be designed to anneal to the unedited version of the spacer sequence. The other set of primers will be designed to flank the desired edit sequence (SEQ ID NO: 7578). A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to the y axis (representing the fluorescent moiety for the probe annealing to SEQ ID NO: 7578), the heterozygotes group near the center of the chart, and the wild type pigs group close to the X axis (representing the fluorescent moiety for the probe annealing to SEQ ID NO: 55).

For the second assay, two sets of primers and two probes will be designed. One set of primers will flank the spacer sequence set forth in SEQ ID NO: 59. A probe, labeled with a fluorescent moiety, will be designed to anneal to the unedited version of the spacer sequence. The other set of primers will be designed to flank the desired edit sequence (SEQ ID NO: 7578)—these may be the same primers and probe used for the first ANP32B assay. A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to the y axis (representing the fluorescent moiety for the probe annealing to SEQ ID NO:P 7578), the heterozygotes group near the center of the chart, and the wild type pigs group close to the X axis (representing the fluorescent moiety for the probe annealing to SEQ ID NO: 59).

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

### REFERENCES

Baker et al., ANP32B, or not to be, that is the question for influenza virus, ELIFE 8:e48084 (2019).
Centers for Disease Control and Prevention (CDC), CDC Estimates of 2009 H1N1 Cases and Related Hospitalizations and Deaths from April 2009 through Mar. 13, 2010, By Age Group, https://www(dot)cdc(dot)gov/h1n1flu/pdf/graph March %202010.pdf (2010).
Centers for Disease Control and Prevention (CDC), What People Who Raise Pigs Need To Know About Influenza (Flu), https://www(dot)cdc(dot)gov/flu/swineflu/people-raise-pigs-flu.htm (2014).
Center for Food Security & Public Health (CFSPH), Swine Influenza, http://www(dot)cfsph(dot)iastate(dot)edu/Factsheets/pdfs/swine_influenza.pdf (2016)
Rajao et al., Pathogenesis and Vaccination of Influenza A Virus in Swine, CURRENT TOPICS IN MICROBIOLOGY AND IMMUNOLOGY 385: 307-326 (2014).
Reilly et al., Cracking the ANP32 whips: Important functions, unequal requirement, and hints at disease implications, BIOESSAYS 36(11):1062-1071 (2014).
Sandbulte et al., Optimal Use of Vaccines for Control of Influenza A Virus in Swine, VACCINES 3:22-73 (2015).
Smith et al., Origins and evolutionary genomics of the 2009 swine-origin HINT influenza A epidemic, NATURE 459:1122-1126 (2009).
Staller et al., ANP32 Proteins Are Essential for Influenza Virus Replication in Human Cells, J. VIROLOGY 93(17): e00217-19 (2019).

All publications cited herein are hereby incorporated by reference, each in their entirety.

1. A pig having a genome comprising a gene edited endogenous ANP32 gene, comprising an ANP32A gene or an ANP32B gene, wherein the ANP32 gene comprises a premature stop codon relative to a wild-type gene.

2. The pig according to claim 1, wherein the premature stop codon is upstream of N129 and D130.

3. The pig according to claim 1, wherein the gene edited endogenous ANP32 gene comprises SEQ ID NO: 7577 or SEQ ID NO: 7578.

4. The pig according to claim 1, wherein the premature stop codon confers resistance to an influenza virus.

5. The pig according to claim 4, wherein the influenza virus comprises a type A influenza virus.

6. The pig according to claim 5, wherein the type A influenza virus comprises an H1N1 subtype virus, an H1N2 subtype virus, or an H3N2 subtype virus.

7. The pig according to claim 1, wherein the pig is heterozygous for the gene edited endogenous ANP32 gene.

8. The pig according to claim 1, wherein the pig is homozygous for the gene edited endogenous ANP32 gene.

9. A cell isolated from the pig of claim 1.

10. An isolated cell line obtained from the pig of claim 1.

11. An isolated cell line according to claim 10, wherein the isolated cell line is an isolated fibroblast line.

12. A pair of guide RNAs (gRNAs) for editing a pig ANP32 gene selected from the group consisting of SEQ ID NO: 33 and SEQ ID NO: 40, SEQ ID NO: 19 and SEQ ID NO: 26, SEQ ID NO: 44 and SEQ ID NO: 46, SEQ ID NO: 55 and SEQ ID NO: 58; and SEQ ID NO: 55 and SEQ ID NO: 59.

13. The pair of gRNAs according to claim 12, wherein the gRNAs comprise sequences consisting of SEQ ID NO: 26 and SEQ ID NO: 19 or SEQ ID NO: 55 and SEQ ID NO: 59.

14. A method of making a pig that is resistant to an influenza virus comprising:
introducing into a pig MII oocyte or zygote a CAS9 protein or a polynucleotide encoding a CAS9 protein and a pair of gRNAs that introduce a premature stop codon into an endogenous ANP32 gene, wherein the premature stop codon is introduced into the endogenous ANP32 gene of the oocyte or zygote;
implanting an embryo obtained from the oocyte or zygote into a recipient female such that a pig is obtained from the implanted embryo, wherein the pig obtained is heterozygous for the ANP32 gene comprising the premature stop codon;
breeding the heterozygous pig to a pig of opposite sex that also comprises the premature stop codon in the ANP32 gene;
selecting offspring from the breeding that are homozygous for the premature stop codon in the ANP32 gene, wherein these homozygous offspring are resistant to influenza.

15. The method of claim 14, wherein the pair of gRNAs comprise sequences consisting of SEQ ID NO: 26 and SEQ ID NO: 19 or SEQ ID NO: 55 and SEQ ID NO: 59.

16. The method of claim 14, wherein the CAS9 protein and the gRNAs are introduced as a pre-formed ribonucleoprotein (RNP) complex.

17. The method of claim 14, wherein the sequence comprising the premature stop codon comprises the nucleic acid sequence consisting of SEQ ID NO: 7577 or SEQ ID NO: 7578.

18-22. (canceled)

23. The pig according to claim 1, wherein the gene edited ANP32 gene encodes an ANP32 protein that does not interact with a Type A influenza virus.
